(19) United States
(12) Patent Application Publication
(10) Pub. No.: US 2004/0044029 A1
(43) Pub. Date: Mar. 4, 2004

(54) Azabicyclic compounds are central nervous system active agents

(76) Inventors: Michael J. Dart, Highland Park, IL (US); Xenia B. Searle, Grayslake, IL (US); Karin Tietje, Mundelein, IL (US); Richard B. Toupence, South Plainfield, NJ (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/219,223

(22) Filed: Aug. 14, 2002

(51) Int. Cl. C 07D 4 1/02; A61K 31/4709

(52) U.S. Cl. 514/307; 514/314; 546/148; 546/176

(57) ABSTRACT

Compounds of formula (I)

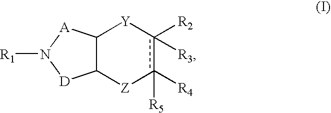

are novel CNS active agents that are useful for treating pain and for treating other disorders associated with the cholinergic system.

### FIELD OF THE INVENTION

The present invention is directed to a series of azabicyclic compounds and a method for treating pain in mammals.

### BACKGROUND OF THE INVENTION

Three major groups of drugs currently used for the treatment of pain include opioids, non-steroidal anti-inflammatory drugs (NSAIDs), and analgesic adjuvants. Opioids such as, but not limited to, morphine act at opioid receptors in the brain and spinal cord to block the transmission of pain signals. However, clinical use of opioids is commonly associated with potential abuse and addiction liabilities, the development of tolerance, and other side effects such as constipation, nausea, and cognitive impairments. NSAIDs typically, but not exclusively, block the production of prostaglandins to prevent sensitization of nerve endings that facilitate the pain signal to the brain. NSAIDs effectively treat mild-to-moderate pain with an inflammatory component, but they have a ceiling effect and are not particularly effective in relieving severe or chronic neuropathic pain. Many commonly prescribed over-the-counter NSAIDs cause gastric distress and bleeding, although the newer COX-2 selective NSAIDs may address these side effects liabilities. Analgesic adjuvants, including certain antidepressants, local anesthetics and anticonvulsants, have been shown to be effective in treating some chromic pain states that have not responded to NSAID or opioid therapy.

A substantial number of medical disorders and conditions produce pain as part of the disorder or condition. Relief of this pain is a major aspect of ameliorating or treating the overall disease or condition. One class of pain reliever may not be effective for a particular patient or group of patients. Therefore, a need exists for novel compounds that treat pain through mechanisms different from the established analgesics.

The compounds of the present invention are novel analgesic compounds that bind to nicotinic acetylcholine receptors. The compounds of the present invention have utility in treating pain and can be administered in combination with an opioid such as, but not limited to, morphine, a non-steroid anti-inflammatory agent such as, but not limited to, aspirin, a tricyclic antidepressant, or an anticonvulsant such as, but not limited to, gabapentin or pregabalin for treating pain. The compounds of the present invention have utility for treating disorders associated with the cholinergic system.

### SUMMARY OF THE INVENTION

The present invention discloses novel azabicyclic compounds and a method for treating pain in mammals. More particularly, the present invention is directed to compounds of formula (I):

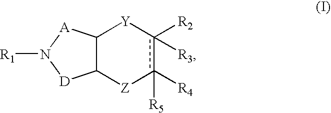

or a pharmaceutically acceptable salt, amide, ester and prodrug thereof, wherein

[symbol] represents a single bond or a double bond;

A is selected from a covalent bond or CH₂;

D is selected from CH₂, CH₂CH₂ or CH₂CH₂CH₂, provided that when D is CH₂CH₂CH₂, then A is a covalent bond;

Y is selected from a covalent bond, CH₂, or CH₂CH₂;

Z is selected from a covalent bond, CH₂, or CH₂CH₂, provided that when Z is CH₂CH₂, then Y is a covalent bond, and further provided that when Y is CH₂CH₂, then Z is a covalent bond;

R₁ is selected from hydrogen, alkoxycarbonyl, alkyl, benzyloxycarbonyl, cyanoalkyl, dihydro-3-pyridinylcarbonyl, hydroxy, hydroxyalkyl, phenoxycarbonyl, —NR₁₀R₁₁, (NR₁₀R₁₁)alkyl or (NR₁₀R₁₁)carbonylalkyl wherein R₁₀ and R₁₁ are independently selected from hydrogen, alkyl or alkylcarbonyl;

R₂ and R₄ are independently selected from hydrogen, aryl or heterocycle, provided that one of R₂ or R₄ is hydrogen; and

R₃ and R₅ are both absent or are independently selected from hydrogen, alkoxy or hydroxy;

provided that when A is a covalent bond, D is CH₂ and Y is a covalent bond, then Z is other than a covalent bond; and

further provided that when A is a covalent bond, D is CH₂ and Z is a covalent bond, then Y is other than a covalent bond.

### DETAILED DESCRIPTION OF THE INVENTION

All patents, patent applications, and literature references cited in the specification are herein incorporated by reference in their entirety.

The present invention is directed to compounds of formula (I):

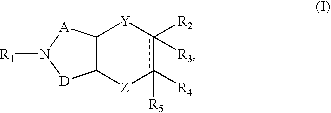

or a pharmaceutically acceptable salt, amide, ester and prodrug thereof, wherein

[symbol] represents a single bond or a double bond;

A is selected from a covalent bond or CH₂;

D is selected from CH₂, CH₂CH₂ or CH₂CH₂CH₂, provided that when D is CH₂CH₂CH₂, then A is a covalent bond;

Y is selected from a covalent bond, CH₂, or CH₂CH₂;

Z is selected from a covalent bond, CH₂, or CH₂CH₂, provided that when Z is CH₂CH₂, then Y is a covalent bond, and further provided that when Y is CH₂CH₂, then Z is a covalent bond;

R₁ is selected from hydrogen, alkoxycarbonyl, alkyl, benzyloxycarbonyl, cyanoalkyl, dihydro-3-pyridinylcarbonyl, hydroxy, hydroxyalkyl, phenoxycarbonyl, —NR₁₀R₁₁, (NR₁₀R₁₁)alkyl or (NR₁₀R₁₁)carbonylalkyl wherein R₁₀ and R₁₁ are independently selected from hydrogen, alkyl or alkylcarbonyl;

R₂ and R₄ are independently selected from hydrogen, aryl or heterocycle, provided that one of R₂ or R₄ is hydrogen; and

R₃ and R₅ are both absent or are independently selected from hydrogen, alkoxy or hydroxy;

provided that when A is a covalent bond, D is CH₂ and Y is a covalent bond, then Z is other than a covalent bond; and

further provided that when A is a covalent bond, D is CH₂ and Z is a covalent bond, then Y is other than a covalent bond.

In another embodiment of the present invention, compounds of formula (II) are disclosed

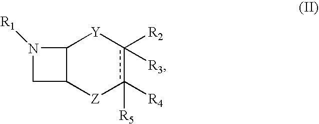

or a pharmaceutically acceptable salt, armide, ester or prodrug thereof wherein Y, Z, R₁, R₂, R₃, R₄ and R₅ are as defined in formula (I).

In another embodiment of the present invention, compounds of formula (II) are disclosed wherein Y is a covalent bond; Z is CH₂; and R₁, R₂, R₃, R₄, and R₅ are as defined in formula (I).

In another embodiment of the present invention, compounds of formula (II) are disclosed wherein [symbol] represents a double bond; Y is a covalent bond; Z is CH₂; R₂ is hydrogen; R₃ and R₅ are absent; R₄ is heterocycle; and R₁ is as defined in formula (I).

In another embodiment of the present invention, compounds of formula (II) are disclosed wherein [symbol] represents a double bond; Y is a covalent bond; Z is CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁, are as defined in formula (I).

In another embodiment of the present invention, compounds of formula (II) are disclosed wherein [symbol] represents a double bond; Y is a covalent bond; Z is CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; and R₁, R₁₀, and R₁₁ are as defined in formula (I).

In another embodiment of the present invention, compounds of formula (II) are disclosed wherein [symbol] represents a double bond; Y is a covalent bond; Z is CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, and nitro; and R₁ is as defined in formula (I).

In another embodiment of the present invention, compounds of formula (II) are disclosed wherein [symbol] represents a double bond; Y is a covalent bond; Z is CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (II) are disclosed wherein [symbol] represents a double bond; Y is a covalent bond; Z is CH₂; R₂ is heterocycle; R₃ and R₅ are absent; R₄ is hydrogen; and R₁ is as defined in formula (I).

In another embodiment of the present invention, compounds of formula (II) are disclosed wherein [symbol] represents a double bond; Y is a covalent bond; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (II) are disclosed wherein [symbol] represents a double bond; Y is a covalent bond; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (II) are disclosed wherein [symbol] represents a double bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, and nitro; R₃ and R₅ are absent; R₄ is hydrogen; and R₁ is as defined in formula (I).

In another embodiment of the present invention, compounds of formula (II) are disclosed wherein [symbol] represents a double bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (II) are disclosed wherein Y is CH₂; Z is a covalent bond; and R₁, R₂, R₃, R₄, and R₅ are as defined in formula (I).

In another embodiment of the present invention, compounds of formula (II) are disclosed wherein [symbol] represents a double bond; Y is CH₂; Z is a covalent bond; R₂ is heterocycle; R₃ and R₅ are absent; R₄ is hydrogen; and R₁ is as defined in formula (I).

In another embodiment of the present invention, compounds of formula (II) are disclosed wherein [symbol] represents a double bond; Y is CH₂; Z is a covalent bond; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (II) are disclosed wherein [symbol] represents a double bond; Y is CH₂; Z is a covalent bond; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (II) are disclosed wherein [symbol] represents a double bond; Y is CH₂; Z is a covalent bond; R₂ is heterocycle wherein the heterocycle is pyridinyl substituted with 0, 1, or 2 substituents independently selected from alkenyl, alkenyloxy, alkoxy, alkyl, alkynyl, cyano, halogen, and nitro; R₃ and R₅ are absent; R₄ is hydrogen; and R₁ is as defined in formula (I).

In another embodiment of the present invention, compounds of formula (II) are disclosed wherein [symbol] represents a double bond; Y is CH₂; Z is a covalent bond; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (II) are disclosed wherein Y is a covalent bond; Z is CH₂CH₂; and R₁, R₂, R₃, R₄, and R₅ are as defined in formula (I).

In another embodiment of the present invention, compounds of formula (II) are disclosed wherein [symbol] represents a double bond; Y is a covalent bond; Z is CH₂CH₂; R₂ is hydrogen; R₃ and R₅ are absent; R₄ is heterocycle; and R₁ is as defined in formula (I).

In another embodiment of the present invention, compounds of formula (II) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁ (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (II) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; and R₁, R₁₀, and R₁₁ are as defined in formula (J).

In another embodiment of the present invention, compounds of formula (II) are disclosed wherein [symbol] represents a double bond; Y is a covalent bond; Z is CH₂CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, and nitro; and R₁ is as defined in formula (I).

In another embodiment of the present invention, compounds of formula (II) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (II) are disclosed wherein [symbol] represents a double bond; Y is a covalent bond; Z is CH₂CH₂; R₂ is heterocycle; R₃ and R₅ are absent; R₄ is hydrogen; and R₁ is as defined in formula (I).

In another embodiment of the present invention, compounds of formula (II) are disclosed wherein [symbol] represents a double bond; Y is a covalent bond; Z is CH₂CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (II) are disclosed wherein [symbol] represents a double bond; Y is a covalent bond; Z is CH₂CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (II) are disclosed wherein [symbol] represents a double bond; Y is a covalent bond; Z is CH₂CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, and nitro; R₃ and R₅ are absent; R₄ is hydrogen; and R₁ is as defined in formula (I).

In another embodiment of the present invention, compounds of formula (II) are disclosed wherein [symbol] represents a double bond; Y is a covalent bond; Z is CH₂CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (II) are disclosed wherein Y is CH₂; Z is CH₂; and R₁, R₂, R₃, R₄, and R₅ are as defined in formula (I).

In another embodiment of the present invention, compounds of formula (II) are disclosed wherein [symbol] represents a double bond; Y is CH₂; Z is CH₂; R₂ is heterocycle; R₃ and R₅ are absent; R₄ is hydrogen; and R₁ is as defined in formula (I).

In another embodiment of the present invention, compounds of formula (II) are disclosed wherein [symbol] represents a double bond; Y is CH₂; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (II) are disclosed wherein [symbol] represents a double bond; Y is CH₂; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, and —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (II) are disclosed wherein [symbol] represents a double bond; Y is CH₂; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, and nitro; R₃ and R₅ are absent; R₄ is hydrogen; and R₁ is as defined in formula (I).

In another embodiment of the present invention, compounds of formula (II) are disclosed wherein [symbol] represents a double bond; Y is CH₂; Z is CH₂; R₂ is heterocycle wherein the heterocycle is selected from 3-pyridinyl, 6-bromo-3-pyridinyl, 6-chloro-3-pyridinyl, 6-fluoro-3-pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (II) are disclosed wherein Y is CH₂CH₂; Z is a covalent bond; and R₁, R₂, R₃, R₄, and R₅ are as defined in formula (I).

Representative compounds of formula (II) include, but are not limited to:

(cis)-4-(3-pyridinyl)-7-azabicyclo[4.2.0]oct-4-ene;

(cis)-4-(6-chloro-3-pyridinyl)-7-azabicyclo[4.2.0]oct-4-ene;

(cis)-4-(6-bromo-3-pyridinyl)-7-azabicyclo[4.2.0]oct-4-ene;

(cis)-4-(6-fluoro-3-pyridinyl)-7-azabicyclo[4.2.0]oct-4-ene;

(cis)-4-(6-methyl-3-pyridinyl)-7-azabicyclo[4.2.0]oct-4-ene;

(cis)-5-(7-azabicyclo[4.2.0]oct-4-en-4-yl)nicotinonitrile;

(cis)-4-(5-methyl-3-pyridinyl)-7-azabicyclo[4.2.0]oct-4-ene;

(cis)-4-(5-chloro-3-pyridinyl)-7-azabicyclo[4.2.0]oct-4-ene;

(cis)-4-(5-fluoro-3-pyridinyl)-7-azabicyclo[4.2.0]oct-4-ene;

(cis)-4-(5-bromo-3-pyridinyl)-7-azabicyclo[4.2.0]oct-4-ene;

(cis)-4-(5-vinyl-3-pyridinyl)-7-azabicyclo[4.2.0]oct-4-ene;

(cis)-4-(5-methoxy-3-pyridinyl)-7-azabicyclo[4.2.0]oct-4-ene;

(cis)-4-[5-(vinyloxy)-3-pyridinyl]-7-azabicyclo[4.2.0]oct-4-ene;

(cis)-4-(5-ethynyl-3-pyridinyl)-7-azabicyclo[4.2.0]oct-4-ene;

(cis)-4-(5,6-dichloro-3-pyridinyl)-7-azabicyclo[4.2.0]oct-4-ene;

(cis)-4-(6-bromo-5-chloro-3-pyridinyl)-7-azabicyclo[4.2.0]oct-4-ene;

(cis)-5-(7-azabicyclo[4.2.0]oct-4-en-4-yl)-2-methylnicotinonitrile;

(cis)-5-(7-azabicyclo[4.2.0]oct-4-en-4-yl)-2-chloronicotinonitrile;

(cis)-5-(7-azabicyclo[4.2.0]oct-4-en-4-yl)-2-bromonicotinonitrile;

(cis)-4-(6-chloro-5-methyl-3-pyridinyl)-7-azabicyclo[4.2.0]oct-4-ene;

(cis)-4-(6-bromo-5-methyl-3-pyridinyl)-7-azabicyclo[4.2.0]oct-4-ene;

(cis)-4-(5-methoxy-6-methyl-3-pyridinyl)-7-azabicyclo[4.2.0]oct-4-ene;

(cis)-4-(6-chloro-5-methoxy-3-pyridinyl)-7-azabicyclo[4.2.0]oct-4-ene;

(cis)-4-(6-bromo-5-methoxy-3-pyridinyl)-7-azabicyclo[4.2.0]oct-4-ene;

(cis)-4-(3-methyl-5-isoxazolyl)-7-azabicyclo[4.2.0]oct-4-ene;

(cis)-2-(7-azabicyclo[4.2.0]oct-4-en-4-yl)furo[3,2-b]pyridine;

(cis)-3-(6-chloro-3-pyridinyl)-6-azabicyclo[3.2.0]hept-3-ene;

(cis)-3-(6-bromo-3-pyridinyl)-6-azabicyclo[3.2.0]hept-3-ene;

(cis)-3-(6-fluoro-3-pyridinyl)-6-azabicyclo[3.2.0]hept-3-ene;

(cis)-3-(6-methyl-3-pyridinyl)-6-azabicyclo[3.2.0]hept-3-ene;

(cis)-5-(6-azabicyclo[3.2.0]hept-3-en-3-yl)nicotinonitrile;

(cis)-3-(5-methyl-3-pyridinyl)-6-azabicyclo[3.2.0]hept-3-ene;

(cis)-3-(5-chloro-3-pyridinyl)-6-azabicyclo[3.2.0]hept-3-ene;

(cis)-3-(5-fluoro-3-pyridinyl)-6-azabicyclo[3.2.0]hept-3-ene;

(cis)-3-(5-bromo-3-pyridinyl)-6-azabicyclo[3.2.0]hept-3-ene;

(cis)-3-(5-vinyl-3-pyridinyl)-6-azabicyclo[3.2.0]hept-3-ene;

(cis)-3-(5-methoxy-3-pyridinyl)-6-azabicyclo[3.2.0]hept-3-ene;

(cis)-3-(5-vinyloxy-3-pyridinyl)-6-azabicyclo[3.2.0]hept-3-ene;

(cis)-3-(5-ethynyl-3-pyridinyl)-6-azabicyclo[3.2.0]hept-3-ene;

(cis)-3-(5,6-dichloro-3-pyridinyl)-6-azabicyclo[3.2.0]hept-3-ene;

(cis)-3-(6-bromo-5-chloro-3-pyridinyl)-6-azabicyclo[3.2.0]hept-3-ene;

(cis)-5-(6-azabicyclo[3.2.0]hept-3-en-3-yl)-2-methylnicotinonitrile;

(cis)-5-(6-azabicyclo[3.2.0]hept-3-en-3-yl)-2-chloronicotinonitrile;

(cis)-5-(6-azabicyclo[3.2.0]hept-3-en-3-yl)-2-bromonicotinonitrile;

(cis)-3-(6-chloro-5-methyl-3-pyridinyl)-6-azabicyclo[3.2.0]hept-3-ene;

(cis)-3-(6-bromo-5-methyl-3-pyridinyl)-6-azabicyclo[3.2.0]hept-3-ene;

(cis)-3-(5-methoxy-6-methyl-3-pyridinyl)-6-azabicyclo[3.2.0]hept-3-ene;

(cis)-3-(6-chloro-5-methoxy-3-pyridinyl)-6-azabicyclo[3.2.0]hept-3-ene;

(cis)-3-(6-bromo-5-methoxy-3-pyridinyl)-6-azabicyclo[3.2.0]hept-3-ene;

(cis)-3-(3-methyl-5-isoxazolyl)-6-azabicyclo[3.2.0]hept-3-ene

(cis)-2-(6-azabicyclo[3.2.0]hept-3-en-3-yl)furo[3,2-b]pyridine;

(cis)-4-(6-chloro-3-pyridinyl)-7-azabicyclo[4.2.0]oct-3-ene;

(cis)-4-(6-bromo-3-pyridinyl)-7-azabicyclo[4.2.0]oct-3-ene;

(cis)-4-(6-fluoro-3-pyridinyl)-7-azabicyclo[4.2.0]oct-3-ene;

(cis)-4-(6-methyl-3-pyridinyl)-7-azabicyclo[4.2.0]oct-3-ene;

(cis)-5-(7-azabicyclo[4.2.0]oct-3-en-4-yl)nicotinonitrile;

(cis)-4-(5-methyl-3-pyridinyl)-7-azabicyclo[4.2.0]oct-3-ene;

(cis)-4-(5-chloro-3-pyridinyl)-7-azabicyclo[4.2.0]oct-3-ene;

(cis)-4-(5-fluoro-3-pyridinyl)-7-azabicyclo[4.2.0]oct-3-ene;

(cis)-4-(5-bromo-3-pyridinyl)-7-azabicyclo[4.2.0]oct-3-ene;

(cis)-4-(5-vinyl-3-pyridinyl)-7-azabicyclo[4.2.0]oct-3-ene;

(cis)-4-(5-methoxy-3-pyridinyl)-7-azabicyclo[4.2.0]oct-3-ene;

(cis)-4-(5-vinyloxy-3-pyridinyl)-7-azabicyclo[4.2.0]oct-3-ene;

(cis)-4-(5-ethynyl-3-pyridinyl)-7-azabicyclo[4.2.0]oct-3-ene;

(cis)-4-(5,6-dichloro-3-pyridinyl)-7-azabicyclo[4.2.0]oct-3-ene;

(cis)-4-(6-bromo-5-chloro-3-pyridinyl)-7-azabicyclo[4.2.0]oct-3-ene;

(cis)-5-(7-azabicyclo[4.2.0]oct-3-en-4-yl)-2-methylnicotinonitrile;

(cis)-5-(7-azabicyclo[4.2.0]oct-3-en-4-yl)-2-chloronicotinonitrile;

(cis)-5-(7-azabicyclo[4.2.0]oct-3-en-4-yl)-2-bromonicotinonitrile;

(cis)-4-(6-chloro-5-methyl-3-pyridinyl)-7-azabicyclo[4.2.0]oct-3-ene;

(cis)-4-(6-bromo-5-methyl-3-pyridinyl)-7-azabicyclo[4.2.0]oct-3-ene;

(cis)-4-(5-methoxy-6-methyl-3-pyridinyl)-7-azabicyclo[4.2.0]oct-3-ene;

(cis)-4-(6-chloro-5-methoxy-3-pyridinyl)-7-azabicyclo[4.2.0]oct-3-ene;

(cis)-4-(6-bromo-5-methoxy-3-pyridinyl)-7-azabicyclo[4.2.0]oct-3-ene;

(cis)-4-(3-methyl-5-isoxazolyl)-7-azabicyclo[4.2.0]oct-3-ene;

(cis)-2-(7-azabicyclo[4.2.0]oct-3-en-4-yl)furo[3,2-b]pyridine;

(cis)-3-(3-pyridinyl)-6-azabicyclo[3.2.0]hept-2-ene;

(cis)-3-(6-chloro-3-pyridinyl)-6-azabicyclo[3.2.0]hept-2-ene;

(cis)-3-(6-bromo-3-pyridinyl)-6-azabicyclo[3.2.0]hept-2-ene;

(cis)-3-(6-fluoro-3-pyridinyl)-6-azabicyclo[3.2.0]hept-2-ene;

(cis)-3-(6-methyl-3-pyridinyl)-6-azabicyclo[3.2.0]hept-2-ene;

(cis)-5-(6-azabicyclo[3.2.0]hept-2-en-3-yl)nicotinonitrile;

(cis)-3-(5-methyl-3-pyridinyl)-6-azabicyclo[3.2.0]hept-2-ene;

(cis)-3-(5-chloro-3-pyridinyl)-6-azabicyclo[3.2.0]hept-2-ene;

(cis)-3-(5-fluoro-3-pyridinyl)-6-azabicyclo[3.2.0]hept-2-ene;

(cis)-3-(5-bromo-3-pyridinyl)-6-azabicyclo[3.2.0]hept-2-ene;

(cis)-3-(5-vinyl-3-pyridinyl)-6-azabicyclo[3.2.0]hept-2-ene;

(cis)-3-(5-methoxy-3-pyridinyl)-6-azabicyclo[3.2.0]hept-2-ene;

(cis)-3-(5-vinyloxy-3-pyridinyl)-6-azabicyclo[3.2.0]hept-2-ene;

(cis)-3-(5-ethynyl-3-pyridinyl)-6-azabicyclo[3.2.0]hept-2-ene;

(cis)-3-(5,6-dichloro-3-pyridinyl)-6-azabicyclo[3.2.0]hept-2-ene;

(cis)-3-(6-bromo-5-chloro-3-pyridinyl)-6-azabicyclo[3.2.0]hept-2-ene;

(cis)-5-(6-azabicyclo[3.2.0]hept-2-en-3-yl)-2-methylnicotinonitrile;

(cis)-5-(6-azabicyclo[3.2.0]hept-2-en-3-yl)-2-chloronicotinonitrile;

(cis)-5-(6-azabicyclo[3.2.0]hept-2-en-3-yl)-2-bromonicotinonitrile;

(cis)-3-(6-chloro-5-methyl-3-pyridinyl)-6-azabicyclo[3.2.0]hept-2-ene;

(cis)-3-(6-bromo-5-methyl-3-pyridinyl)-6-azabicyclo[3.2.0]hept-2-ene;

(cis)-3-(5-methoxy-6-methyl-3-pyridinyl)-6-azabicyclo[3.2.0]hept-2-ene;

(cis)-3-(6-chloro-5-methoxy-3-pyridinyl)-6-azabicyclo[3.2.0]hept-2-ene;

(cis)-3-(6-bromo-5-methoxy-3-pyridinyl)-6-azabicyclo[3.2.0]hept-2-ene;

(cis)-3-(3-methyl-5-isoxazolyl)-6-azabicyclo[3.2.0]hept-2-ene;

(cis)-2-(6-azabicyclo[3.2.0]hept-2-en-3-yl)furo[3,2-b]pyridine;

(cis)-5-(3-pyridinyl)-7-azabicyclo[4.2.0]oct-4-ene;

(cis)-5-(6-chloro-3-pyridinyl)-7-azabicyclo[4.2.0]oct-4-ene;

(cis)-5-(6-bromo-3-pyridinyl)-7-azabicyclo[4.2.0]oct-4-ene;

(cis)-5-(6-fluoro-3-pyridinyl)-7-azabicyclo[4.2.0]oct-4-ene;

(cis)-5-(6-methyl-3-pyridinyl)-7-azabicyclo[4.2.0]oct-4-ene;

(cis)-5-(7-azabicyclo[4.2.0]oct-4-en-5-yl)nicotinonitrile;

(cis)-5-(5-methyl-3-pyridinyl)-7-azabicyclo[4.2.0]oct-4-ene;

(cis)-5-(5-chloro-3-pyridinyl)-7-azabicyclo[4.2.0]oct-4-ene;

(cis)-5-(5-fluoro-3-pyridinyl)-7-azabicyclo[4.2.0]oct-4-ene;

(cis)-5-(5-bromo-3-pyridinyl)-7-azabicyclo[4.2.0]oct-4-ene;

(cis)-5-(5-vinyl-3-pyridinyl)-7-azabicyclo[4.2.0]oct-4-ene;

(cis)-5-(5-methoxy-3-pyridinyl)-7-azabicyclo[4.2.0]oct-4-ene;

(cis)-5-(5-vinyloxy-3-pyridinyl)-7-azabicyclo[4.2.0]oct-4-ene;

(cis)-5-(5-ethynyl-3-pyridinyl)-7-azabicyclo[4.2.0]oct-4-ene;

(cis)-5-(5,6-dichloro-3-pyridinyl)-7-azabicyclo[4.2.0]oct-4-ene;

(cis)-5-(6-bromo-5-chloro-3-pyridinyl)-7-azabicyclo[4.2.0]oct-4-ene;

(cis)-5-(7-azabicyclo[4.2.0]oct-4-en-5-yl)-2-methylnicotinonitrile;

(cis)-5-(7-azabicyclo[4.2.0]oct-4-en-5-yl)-2-bromonicotinonitrile;

(cis)-5-(7-azabicyclo[4.2.0]oct-4-en-5-yl)-2-chloronicotinonitrile;

(cis)-5-(6-chloro-5-methyl-3-pyridinyl)-7-azabicyclo[4.2.0]oct-4-ene;

(cis)-5-(6-bromo-5-methyl-3-pyridinyl)-7-azabicyclo[4.2.0]oct-4-ene;

(cis)-5-(5-methoxy-6-methyl-3-pyridinyl)-7-azabicyclo[4.2.0]oct-4-ene;

(cis)-5-(6-chloro-5-methoxy-3-pyridinyl)-7-azabicyclo[4.2.0]oct-4-ene;

(cis)-5-(6-bromo-5-methoxy-3-pyridinyl)-7-azabicyclo[4.2.0]oct-4-ene;

(cis)-2-(7-azabicyclo[4.2.0]oct-4-en-5-yl)furo[3,2-b]pyridine;

(cis)-5-(3-methyl-5-isoxazolyl)-7-azabicyclo[4.2.0]oct-4-ene;

(cis)-4-(3-pyridinyl)-6-azabicyclo[3.2.0]hept-3-ene;

(cis)-4-(6-chloro-3-pyridinyl)-6-azabicyclo[3.2.0]hept-3-ene;

(cis)-4-(6-bromo-3-pyridinyl)-6-azabicyclo[3.2.0]hept-3-ene;

(cis)-4-(6-fluoro-3-pyridinyl)-6-azabicyclo[3.2.0]hept-3-ene;

(cis)-4-(6-methyl-3-pyridinyl)-6-azabicyclo[3.2.0]hept-3-ene;

(cis)-5-(6-azabicyclo[3.2.0]hept-3-en-4-yl)nicotinonitrile;

(cis)-4-(5-methyl-3-pyridinyl)-6-azabicyclo[3.2.0]hept-3-ene;

(cis)-4-(5-chloro-3-pyridinyl)-6-azabicyclo[3.2.0]hept-3-ene;

(cis)-4-(5-fluoro-3-pyridinyl)-6-azabicyclo[3.2.0]hept-3-ene;

(cis)-4-(5-bromo-3-pyridinyl)-6-azabicyclo[3.2.0]hept-3-ene;

(cis)-4-(5-vinyl-3-pyridinyl)-6-azabicyclo[3.2.0]hept-3-ene;

(cis)-4-(5-methoxy-3-pyridinyl)-6-azabicyclo[3.2.0]hept-3-ene;

(cis)-4-(5-vinyloxy-3-pyridinyl)-6-azabicyclo[3.2.0]hept-3-ene;

(cis)-4-(5-ethynyl-3-pyridinyl)-6-azabicyclo[3.2.0]hept-3-ene;

(cis)-4-(5,6-dichloro-3-pyridinyl)-6-azabicyclo[3.2.0]hept-3-ene;

(cis)-4-(6-bromo-5-chloro-3-pyridinyl)-6-azabicyclo[3.2.0]hept-3-ene;

(cis)-5-(6-azabicyclo[3.2.0]hept-3-en-4-yl)-2-methylnicotinonitrile;

(cis)-5-(6-azabicyclo[3.2.0]hept-3-en-4-yl)-2-chloronicotinonitrile;

(cis)-5-(6-azabicyclo[3.2.0]hept-3-en-4-yl)-2-bromonicotinonitrile;

(cis)-4-(6-chloro-5-methyl-3-pyridinyl)-6-azabicyclo[3.2.0]hept-3-ene;

(cis)-4-(6-bromo-5-methyl-3-pyridinyl)-6-azabicyclo[3.2.0]hept-3-ene;

(cis)-4-(5-methoxy-6-methyl-3-pyridinyl)-6-azabicyclo[3.2.0]hept-3-ene;

(cis)-4-(6-chloro-5-methoxy-3-pyridinyl)-6-azabicyclo[3.2.0]hept-3-ene;

(cis)-4-(6-bromo-5-methoxy-3-pyridinyl)-6-azabicyclo[3.2.0]hept-3-ene;

(cis)-4-(3-methyl-5-isoxazolyl)-6-azabicyclo[3.2.0]hept-3-ene;

(cis)-2-(6-azabicyclo[3.2.0]hept-3-en-4-yl)furo[3,2-b]pyridine; or a pharmaceutically acceptable salt, amide, ester or prodrug thereof.

In another embodiment of the present invention, compounds of formula (III) are disclosed

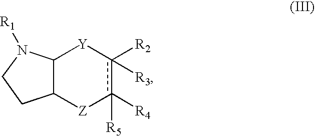

or a pharmaceutically acceptable salt, amide, ester or prodrug thereof wherein Y, Z, R₁, R₂, R₃, R₄, and R₅ are as defined in formula (I).

In another embodiment of the present invention, compounds of formula (III) are disclosed wherein Y is a covalent bond; Z is a covalent bond; and R₁, R₂, R₃, R₄, and R₅ are as defined in formula (I).

In another embodiment of the present invention, compounds of formula (III) are disclosed wherein Y is a covalent bond; Z is CH₂; and R₁, R₂, R₃, R₄, and R₅ are as defined in formula (I).

In another embodiment of the present invention, compounds of formula (III) are disclosed wherein [symbol] represents a double bond; Y is a covalent bond; Z is CH₂; R₂ is hydrogen; R₃ and R₅ are absent; R₄ is heterocycle; and R₁ is as defined in formula (I).

In another embodiment of the present invention, compounds of formula (III) are disclosed wherein [symbol] represents a double bond; Y is a covalent bond; Z is CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁R)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is a covalent bond; Z is CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (III) are disclosed wherein [symbol] represents a double bond; Y is a covalent bond; Z is CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (III) are disclosed wherein [symbol] represents a double bond; Y is a covalent bond; Z is CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (III) are disclosed wherein [symbol] represents a double bond; Y is a covalent bond; Z is CH₂; R₂ is heterocycle; R₃ and R₅ are absent; R₄ is hydrogen; and R₁ is as defined in formula (I).

In another embodiment of the present invention, compounds of formula (III) are disclosed wherein [symbol] represents a double bond; Y is a covalent bond; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is a covalent bond; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (III) are disclosed wherein [symbol] represents a double bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (III) are disclosed wherein [symbol] represents a double bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (III) are disclosed wherein Y is CH₂; Z is a covalent bond; and R₁, R₂, R₃, R₄, and R₅ are as defined in formula (I).

In another embodiment of the present invention, compounds of formula (III) are disclosed wherein [symbol] represents a double bond; Y is CH₂; Z is a covalent bond; R₂ is heterocycle; R₃ and R₅ are absent; R₄ is hydrogen; and R₁ is as defined in formula (I).

In another embodiment of the present invention, compounds of formula (III) are disclosed wherein [symbol] represents a double bond; Y is CH₂; Z is a covalent bond; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is CH₂; Z is a covalent bond; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (III) are disclosed wherein [symbol] represents a double bond; Y is CH₂; Z is a covalent bond; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (III) are disclosed wherein [symbol] represents a double bond; Y is CH₂; Z is a covalent bond; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (III) are disclosed wherein Y is a covalent bond; Z is CH₂CH₂; and R₁, R₂, R₃, R₄ and R₅ are as defined in formula (I).

In another embodiment of the present invention, compounds of formula (III) are disclosed wherein [symbol] represents a double bond; Y is a covalent bond; Z is CH₂CH₂; R₂ is hydrogen; R₃ and R₅ are absent; R₄ is heterocycle; and R₁ is as defined in formula (I).

In another embodiment of the present invention, compounds of formula (III) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (III) are disclosed wherein [symbol] represents a double bond; Y is a covalent bond; Z is CH₂CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (III) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (III) are disclosed wherein [symbol] represents a double bond; Y is a covalent bond; Z is CH₂CH₂; R₂ is heterocycle; R₃ and R₅ are absent; R₄ is hydrogen; and R₁ is as defined in formula (I).

In another embodiment of the present invention, compounds of formula (III) are disclosed wherein [symbol] represents a double bond; Y is a covalent bond; Z is CH₂CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is a covalent bond; Z is CH₂CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (III) are disclosed wherein [symbol] represents a double bond; Y is a covalent bond; Z is CH₂CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (III) are disclosed wherein [symbol] represents a double bond; Y is a covalent bond; Z is CH₂CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (III) are disclosed wherein Y is CH₂; Z is CH₂; and R₁, R₂, R₃, R₄, and R₅ are as defined in formula (I).

In another embodiment of the present invention, compounds of formula (III) are disclosed wherein [symbol] represents a double bond; Y is CH₂; Z is CH₂; R₂ is heterocycle; R₃ and R₅ are absent; R₄ is hydrogen; and R₁ is as defined in formula (I).

In another embodiment of the present invention, compounds of formula (III) are disclosed wherein [symbol] represents a double bond; Y is CH₂; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(O)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (III) are disclosed wherein [symbol] represents a double bond; Y is CH₂; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (III) are disclosed wherein [symbol] represents a double bond; Y is CH₂; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (III) are disclosed wherein [symbol] represents a double bond; Y is CH₂; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (III) are disclosed wherein Y is CH₂CH₂; Z is a covalent bond; and R₁, R₂, R₃, R₄, and R₅ are as defined in formula (I).

Representative compounds of formula (III) include, but are not limited to:

(cis)-5-(3-pyridinyl)-1,2,3,3a,4,6a-hexahydrocyclopenta[b]pyrrole;

(cis)-5-(3-pyridinyl)-1,2,3,3a,6,6a-hexahydrocyclopenta[b]pyrrole;

(cis)-6-(3-pyridinyl)-2,3,3a,4,5,7a-hexahydro-1H-indole;

(cis)-6-(6-chloro-3-pyridinyl)-2,3,3a,4,5,7a-hexahydro-1H-indole;

(cis)-6-(6-bromo-3-pyridinyl)-2,3,3a,4,5,7a-hexahydro-1H-indole;

(cis)-6-(6-fluoro-3-pyridinyl)-2,3,3a,4,5,7a-hexahydro-1H-indole;

(cis)-6-(6-methyl-3-pyridinyl)-2,3,3a,4,5,7a-hexahydro-1H-indole;

(cis)-5-(2,3,3a,4,5,7a-hexahydro-1H-indol-6-yl)nicotinonitrile;

(cis)-6-(5-methyl-3-pyridinyl)-2,3,3a,4,5,7a-hexahydro-1H-indole;

(cis)-6-(5-chloro-3-pyridinyl)-2,3,3a,4,5,7a-hexahydro-1H-indole;

(cis)-6-(5-fluoro-3-pyridinyl)-2,3,3a,4,5,7a-hexahydro-1H-indole;

(cis)-6-(5-bromo-3-pyridinyl)-2,3,3a,4,5,7a-hexahydro-1H-indole;

(cis)-6-(5-vinyl-3-pyridinyl)-2,3,3a,4,5,7a-hexahydro-1H-indole;

(cis)-6-(5-methoxy-3-pyridinyl)-2,3,3a,4,5,7a-hexahydro-1H-indole;

(cis)-6-(5-vinyloxy-3-pyridinyl)-2,3,3a,4,5,7a-hexahydro-1H-indole;

(cis)-6-(5-ethynyl-3-pyridinyl)-2,3,3a,4,5,7a-hexahydro-1H-indole;

(cis)-6-(5,6-dichloro-3-pyridinyl)-2,3,3a,4,5,7a-hexahydro-1H-indole;

(cis)-6-(6-bromo-5-chloro-3-pyridinyl)-2,3,3a,4,5,7a-hexahydro-1H-indole;

(cis)-5-(2,3,3a,4,5,7a-hexahydro-1H-indol-6-yl)-2-methylnicotinonitrile;

(cis)-2-chloro-5-(2,3,3a,4,5,7a-hexahydro-1H-indol-6-yl)nicotinonitrile;

(cis)-2-bromo-5-(2,3,3a,4,5,7a-hexahydro-1H-indol-6-yl)nicotinonitrile;

(cis)-6-(6-chloro-5-methyl-3-pyridinyl)-2,3,3a,4,5,7a-hexahydro-1H-indole;

(cis)-6-(6-bromo-5-methyl-3-pyridinyl)-2,3,3a,4,5,7a-hexahydro-1H-indole;

(cis)-6-(5-methoxy-6-methyl-3-pyridinyl)-2,3,3a,4,5,7a-hexahydro-1H-indole;

(cis)-6-(6-chloro-5-methoxy-3-pyridinyl)-2,3,3a,4,5,7a-hexahydro-1H-indole;

(cis)-6-(6-bromo-5-methoxy-3-pyridinyl)-2,3,3a,4,5,7a-hexahydro-1H-indole;

(cis)-6-(3-methyl-5-isoxazolyl)-2,3,3a,4,5,7a-hexahydro-1H-indole;

(cis)-2-(2,3,3a,4,5,7a-hexahydro-1H-indol-6-yl)furo[3,2-b]pyridine;

(cis)-5-(6-chloro-3-pyridinyl)-1,2,3,3a,4,6a-hexahydrocyclopenta[b]pyrrole;

(cis)-5-(6-bromo-3-pyridinyl)-1,2,3,3a,4,6a-hexahydrocyclopenta[b]pyrrole;

(cis)-5-(6-fluoro-3-pyridinyl)-1,2,3,3a,4,6a-hexahydrocyclopenta[b]pyrrole;

(cis)-5-(6-methyl-3-pyridinyl)-1,2,3,3a,4,6a-hexahydrocyclopenta[b]pyrrole;

(cis)-5-(1,2,3,3a,4,6a-hexahydrocyclopenta[b]pyrrol-5-yl)nicotinonitrile

(cis)-5-(5-methyl-3-pyridinyl)-1,2,3,3a,4,6a-hexahydrocyclopenta[b]pyrrole;

(cis)-5-(5-chloro-3-pyridinyl)-1,2,3,3a,4,6a-hexahydrocyclopenta[b]pyrrole;

(cis)-5-(5-fluoro-3-pyridinyl)-1,2,3,3a,4,6a-hexahydrocyclopenta[b]pyrrole;

(cis)-5-(5-bromo-3-pyridinyl)-1,2,3,3a,4,6a-hexahydrocyclopenta[b]pyrrole;

(cis)-5-(5-vinyl-3-pyridinyl)-1,2,3,3a,4,6a-hexahydrocyclopenta[b]pyrrole;

(cis)-5-(5-methoxy-3-pyridinyl)-1,2,3,3a,4,6a-hexahydrocyclopenta[b]pyrrole;

(cis)-5-(5-vinyloxy-3-pyridinyl)-1,2,3,3a,4,6a-hexahydrocyclopenta[b]pyrrole;

(cis)-5-(5-ethynyl-3-pyridinyl)-1,2,3,3a,4,6a-hexahydrocyclopenta[b]pyrrole;

(cis)-5-(5,6-dichloro-3-pyridinyl)-1,2,3,3a,4,6a-hexahydrocyclopenta[b]pyrrole;

(cis)-5-(6-bromo-5-chloro-3-pyridinyl)-1,2,3,3a,4,6a-hexahydrocyclopenta[b]pyrrole;

(cis)-5-(1,2,3,3a,4,6a-hexahydrocyclopenta[b]pyrrol-5-yl)-2-methylnicotinonitrile

(cis)-2-chloro-5-(1,2,3,3a,4,6a-hexahydrocyclopenta[b]pyrrol-5-yl)nicotinonitrile

(cis)-2-bromo-5-(1,2,3,3a,4,6a-hexahydrocyclopenta[b]pyrrol-5-yl)nicotinonitrile

(cis)-5-(6-chloro-5-methyl-3-pyridinyl)-1,2,3,3a,4,6a-hexahydrocyclopenta[b]pyrrole;

(cis)-5-(6-bromo-5-methyl-3-pyridinyl)-1,2,3,3a,4,6a-hexahydrocyclopenta[b]pyrrole;

(cis)-5-(5-methoxy-6-methyl-3-pyridinyl)-1,2,3,3a,4,6a-hexahydrocyclopenta[b]pyrrole;

(cis)-5-(6-chloro-5-methoxy-3-pyridinyl)-1,2,3,3a,4,6a-hexahydrocyclopenta[b]pyrrole;

(cis)-5-(6-bromo-5-methoxy-3-pyridinyl)-1,2,3,3a,4,6a-hexahydrocyclopenta[b]pyrrole;

(cis)-5-(3-methyl-5-isoxazolyl)-1,2,3,3a,4,6a-hexahydrocyclopenta[b]pyrrole;

(cis)-2-(1,2,3,3a,4,6a-hexahydrocyclopenta[b]pyrrol-5-yl)furo[3,2-b]pyridine;

(cis)-6-(3-pyridinyl)-2,3,3a,4,7,7a-hexahydro-1H-indole;

(cis)-6-(6-chloro-3-pyridinyl)-2,3,3a,4,7,7a-hexahydro-1H-indole;

(cis)-6-(6-bromo-3-pyridinyl)-2,3,3a,4,7,7a-hexahydro-1H-indole;

(cis)-6-(6-fluoro-3-pyridinyl)-2,3,3a,4,7,7a-hexahydro-1H-indole;

(cis)-6-(6-methyl-3-pyridinyl)-2,3,3a,4,7,7a-hexahydro-1H-indole;

(cis)-5-(2,3,3a,4,7,7a-hexahydro-1H-indol-6-yl)nicotinonitrile;

(cis)-6-(5-methyl-3-pyridinyl)-2,3,3a,4,7,7a-hexahydro-1H-indole;

(cis)-6-(5-chloro-3-pyridinyl)-2,3,3a,4,7,7a-hexahydro-1H-indole;

(cis)-6-(5-fluoro-3-pyridinyl)-2,3,3a,4,7,7a-hexahydro-1H-indole;

(cis)-6-(5-bromo-3-pyridinyl)-2,3,3a,4,7,7a-hexahydro-1H-indole;

(cis)-6-(5-vinyl-3-pyridinyl)-2,3,3a,4,7,7a-hexahydro-1H-indole;

(cis)-6-(5-methoxy-3-pyridinyl)-2,3,3a,4,7,7a-hexahydro-1H-indole;

(cis)-6-(5-vinyloxy-3-pyridinyl)-2,3,3a,4,7,7a-hexahydro-1H-indole;

(cis)-6-(5-ethynyl-3-pyridinyl)-2,3,3a,4,7,7a-hexahydro-1H-indole;

(cis)-6-(5,6-dichloro-3-pyridinyl)-2,3,3a,4,7,7a-hexahydro-1H-indole;

(cis)-6-(6-bromo-5-chloro-3-pyridinyl)-2,3,3a,4,7,7a-hexahydro-1H-indole;

(cis)-5-(2,3,3a,4,7,7a-hexahydro-1H-indol-6-yl)-2-methylnicotinonitrile;

(cis)-2-chloro-5-(2,3,3a,4,7,7a-hexahydro-1H-indol-6-yl)nicotinonitrile;

(cis)-2-bromo-5-(2,3,3a,4,7,7a-hexahydro-1H-indol-6-yl)nicotinonitrile;

(cis)-6-(6-chloro-5-methyl-3-pyridinyl)-2,3,3a,4,7,7a-hexahydro-1H-indole;

(cis)-6-(6-bromo-5-methyl-3-pyridinyl)-2,3,3a,4,7,7a-hexahydro-1H-indole;

(cis)-6-(5-methoxy-6-methyl-3-pyridinyl)-2,3,3a,4,7,7a-hexahydro-1H-indole;

(cis)-6-(6-chloro-5-methoxy-3-pyridinyl)-2,3,3a,4,7,7a-hexahydro-1H-indole;

(cis)-6-(6-bromo-5-methoxy-3-pyridinyl)-2,3,3a,4,7,7a-hexahydro-1H-indole;

(cis)-6-(3-methyl-5-isoxazolyl)-2,3,3a,4,7,7a-hexahydro-1H-indole;

(cis)-2-(2,3,3a,4,7,7a-hexahydro-1H-indol-6-yl)furo[3,2-b]pyridine;

(cis)-5-(3-pyridinyl)-1,2,3,3a,6,6a-hexahydrocyclopenta[b]pyrrole;

(cis)-5-(6-chloro-3-pyridinyl)-1,2,3,3a,6,6a-hexahydrocyclopenta[b]pyrrole;

(cis)-5-(6-bromo-3-pyridinyl)-1,2,3,3a,6,6a-hexahydrocyclopenta[b]pyrrole;

(cis)-5-(6-fluoro-3-pyridinyl)-1,2,3,3a,6,6a-hexahydrocyclopenta[b]pyrrole;

(cis)-5-(6-methyl-3-pyridinyl)-1,2,3,3a,6,6a-hexahydrocyclopenta[b]pyrrole;

(cis)-5-(1,2,3,3a,6,6a-hexahydrocyclopenta[b]pyrrol-5-yl)nicotinonitrile;

(cis)-5-(5-methyl-3-pyridinyl)-1,2,3,3a,6,6a-hexahydrocyclopenta[b]pyrrole;

(cis)-5-(5-chloro-3-pyridinyl)-1,2,3,3a,6,6a-hexahydrocyclopenta[b]pyrrole;

(cis)-5-(5-fluoro-3-pyridinyl)-1,2,3,3a,6,6a-hexahydrocyclopenta[b]pyrrole;

(cis)-5-(5-bromo-3-pyridinyl)-1,2,3,3a,6,6a-hexahydrocyclopenta[b]pyrrole;

(cis)-5-(5-vinyl-3-pyridinyl)-1,2,3,3a,6,6a-hexahydrocyclopenta[b]pyrrole;

(cis)-5-(5-methoxy-3-pyridinyl)-1,2,3,3a,6,6a-hexahydrocyclopenta[b]pyrrole;

(cis)-5-(5-vinyloxy-3-pyridinyl)-1,2,3,3a,6,6a-hexahydrocyclopenta[b]pyrrole;

(cis)-5-(5-ethynyl-3-pyridinyl)-1,2,3,3a,6,6a-hexahydrocyclopenta[b]pyrrole;

(cis)-5-(5,6-dichloro-3-pyridinyl)-1,2,3,3a,6,6a-hexahydrocyclopenta[b]pyrrole;

(cis)-5-(6-bromo-5-chloro-3-pyridinyl)-1,2,3,3a,6,6a-hexahydrocyclopenta[b]pyrrole;

(cis)-5-(1,2,3,3a,6,6a-hexahydrocyclopenta[b]pyrrol-5-yl)-2-methylnicotinonitrile;

(cis)-2-chloro-5-(1,2,3,3a,6,6a-hexahydrocyclopenta[b]pyrrol-5-yl)nicotinonitrile;

(cis)-2-bromo-5-(1,2,3,3a,6,6a-hexahydrocyclopenta[b]pyrrol-5-yl)nicotinonitrile;

(cis)-5-(6-chloro-5-methyl-3-pyridinyl)-1,2,3,3a,6,6a-hexahydrocyclopenta[b]pyrrole;

(cis)-5-(6-bromo-5-methyl-3-pyridinyl)-1,2,3,3a,6,6a-hexahydrocyclopenta[b]pyrrole;

(cis)-5-(5-methoxy-6-methyl-3-pyridinyl)-1,2,3,3a,6,6a-hexahydrocyclopenta[b]pyrrole;

(cis)-5-(6-chloro-5-methoxy-3-pyridinyl)-1,2,3,3a,6,6a-hexahydrocyclopenta[b]pyrrole;

(cis)-5-(6-bromo-5-methoxy-3-pyridinyl)-1,2,3,3a,6,6a-hexahydrocyclopenta[b]pyrrole;

(cis)-5-(3-methyl-5-isoxazolyl)-1,2,3,3a,6,6a-hexahydrocyclopenta[b]pyrrole;

(cis)-2-(1,2,3,3a,6,6a-hexahydrocyclopenta[b]pyrrol-5-yl)furo[3,2-b]pyridine;

(cis)-7-(3-pyridinyl)-2,3,3a,4,5,7a-hexahydro-1H-indole;

(cis)-7-(6-chloro-3-pyridinyl)-2,3,3a,4,5,7a-hexahydro-1H-indole;

(cis)-7-(6-bromo-3-pyridinyl)-2,3,3a,4,5,7a-hexahydro-1H-indole;

(cis)-7-(6-fluoro-3-pyridinyl)-2,3,3a,4, 5,7a-hexahydro-1H-indole;

(cis)-7-(6-methyl-3-pyridinyl)-2,3,3a,4,5,7a-hexahydro-1H-indole;

(cis)-5-(2,3,3a,4,5,7a-hexahydro-1H-indol-7-yl)nicotinonitrile;

(cis)-7-(5-methyl-3-pyridinyl)-2,3,3a,4,5,7a-hexahydro-1H-indole;

(cis)-7-(5-chloro-3-pyridinyl)-2,3,3a,4, 5,7a-hexahydro-1H-indole;

(cis)-7-(5-fluoro-3-pyridinyl)-2,3,3a,4, 5,7a-hexahydro-1H-indole;

(cis)-7-(5-bromo-3-pyridinyl)-2,3,3a,4,5,7a-hexahydro-1H-indole;

(cis)-7-(5-vinyl-3-pyridinyl)-2,3,3a,4,5,7a-hexahydro-1H-indole;

(cis)-7-(5-methoxy-3-pyridinyl)-2,3,3a,4,5,7a-hexahydro-1H-indole;

(cis)-7-(5-vinyloxy-3-pyridinyl)-2,3,3a,4,5,7a-hexahydro-1H-indole;

(cis)-7-(5-ethynyl-3-pyridinyl)-2,3,3a,4,5,7a-hexahydro-1H-indole;

(cis)-7-(5,6-dichloro-3-pyridinyl)-2,3,3a,4,5,7a-hexahydro-1H-indole;

(cis)-7-(6-bromo-5-chloro-3-pyridinyl)-2,3,3a,4,5,7a-hexahydro-1H-indole;

(cis)-5-(2,3,3a,4,5,7a-hexahydro-1H-indol-7-yl)-2-methylnicotinonitrile;

(cis)-2-chloro-5-(2,3,3a,4,5,7a-hexahydro-1H-indol-7-yl)nicotinonitrile;

(cis)-2-bromo-5-(2,3,3a,4,5,7a-hexahydro-1H-indol-7-yl)nicotinonitrile;

(cis)-7-(6-chloro-5-methyl-3-pyridinyl)-2,3,3a,4,5,7a-hexahydro-1H-indole;

(cis)-7-(6-bromo-5-methyl-3-pyridinyl)-2,3,3a,4,5,7a-hexahydro-1H-indole;

(cis)-7-(5-methoxy-6-methyl-3-pyridinyl)-2,3,3a,4,5,7a-hexahydro-1H-indole;

(cis)-7-(6-chloro-5-methoxy-3-pyridinyl)-2,3,3a,4,5,7a-hexahydro-1H-indole;

(cis)-7-(6-bromo-5-methoxy-3-pyridinyl)-2,3,3a,4,5,7a-hexahydro-1H-indole;

(cis)-2-(2,3,3a,4,5,7a-hexahydro-1H-indol-7-yl)furo[3,2-b]pyridine;

(cis)-7-(3-methyl-5-isoxazolyl)-2,3,3a,4,5,7a-hexahydro-1H-indole;

(cis)-6-(3-pyridinyl)-1,2,3,3a,4,6a-hexahydrocyclopenta[b]pyrrole;

(cis)-6-(6-chloro-3-pyridinyl)-1,2,3,3a,4,6a-hexahydrocyclopenta[b]pyrrole;

(cis)-6-(6-bromo-3-pyridinyl)-1,2,3,3a,4,6a-hexahydrocyclopenta[b]pyrrole;

(cis)-6-(6-fluoro-3-pyridinyl)-1,2,3,3a,4,6a-hexahydrocyclopenta[b]pyrrole;

(cis)-6-(6-methyl-3-pyridinyl)-1,2,3,3a,4,6a-hexahydrocyclopenta[b]pyrrole;

(cis)-5-(1,2,3,3a,4,6a-hexahydrocyclopenta[b]pyrrol-6-yl)nicotinonitrile;

(cis)-6-(5-methyl-3-pyridinyl)-1,2,3,3a,4,6a-hexahydrocyclopenta[b]pyrrole;

(cis)-6-(5-chloro-3-pyridinyl)-1,2,3,3a,4,6a-hexahydrocyclopenta[b]pyrrole;

(cis)-6-(5-fluoro-3-pyridinyl)-1,2,3,3a,4,6a-hexahydrocyclopenta[b]pyrrole;

(cis)-6-(5-bromo-3-pyridinyl)-1,2,3,3a,4,6a-hexahydrocyclopenta[b]pyrrole;

(cis)-6-(5-vinyl-3-pyridinyl)-1,2,3,3a,4,6a-hexahydrocyclopenta[b]pyrrole;

(cis)-6-(5-vinyloxy-3-pyridinyl)-1,2,3,3a,4,6a-hexahydrocyclopenta[b]pyrrole;

(cis)-6-(5-ethynyl-3-pyridinyl)-1,2,3,3a,4,6a-hexahydrocyclopenta[b]pyrrole;

(cis)-6-(5,6-dichloro-3-pyridinyl)-1,2,3,3a,4,6a-hexahydrocyclopenta[b]pyrrole;

(cis)-6-(6-bromo-5-chloro-3-pyridinyl)-1,2,3,3a,4,6a-hexahydrocyclopenta[b]pyrrole;

(cis)-5-(1,2,3,3a,4,6a-hexahydrocyclopenta[b]pyrrol-6-yl)-2-methylnicotinonitrile;

(cis)-2-chloro-5-(1,2,3,3a,4,6a-hexahydrocyclopenta[b]pyrrol-6-yl)nicotinonitrile;

(cis)-2-bromo-5-(1,2,3,3a,4,6a-hexahydrocyclopenta[b]pyrrol-6-yl)nicotinonitrile;

(cis)-6-(6-chloro-5-methyl-3-pyridinyl)-1,2,3,3a,4,6a-hexahydrocyclopenta[b]pyrrole;

(cis)-6-(6-bromo-5-methyl-3-pyridinyl)-1,2,3,3a,4,6a-hexahydrocyclopenta[b]pyrrole;

(cis)-6-(5-methoxy-6-methyl-3-pyridinyl)-1,2,3,3a,4,6a-hexahydrocyclopenta[b]pyrrole;

(cis)-6-(6-chloro-5-methoxy-3-pyridinyl)-1,2,3,3a,4,6a-hexahydrocyclopenta[b]pyrrole;

(cis)-6-(6-bromo-5-methoxy-3-pyridinyl)-1,2,3,3a,4,6a-hexahydrocyclopenta[b]pyrrole;

(cis)-6-(3-methyl-5-isoxazolyl)-1,2,3,3a,4,6a-hexahydrocyclopenta[b]pyrrole;

(cis)-2-(1,2,3,3a,4,6a-hexahydrocyclopenta[b]pyrrol-6-yl)furo[3,2-b]pyridine; or a pharmaceutically acceptable salt, amide, ester and prodrugs thereof.

In another embodiment of the present invention, compounds of formula (IV) are disclosed

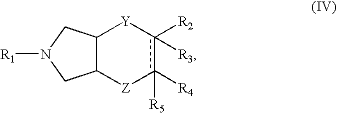

or a pharmaceutically acceptable salt, amide, ester or prodrugs thereof wherein Y, Z, R₁, R₂, R₃, R₄ and R₅ are as defined in formula (I).

In another embodiment of the present invention, compounds of formula (IV) are disclosed wherein Y is a covalent bond; Z is a covalent bond; and R₁, R₂, R₃, R₄, and R₅ are as defined in formula (I).

In another embodiment of the present invention, compounds of formula (IV) are disclosed wherein [symbol] represents a double bond; Y is a covalent bond; Z is a covalent bond; R₂ is hydrogen; R₃ and R₅ are absent; R₄ is heterocycle; and R₁ is as defined in formula (I).

In another embodiment of the present invention, compounds of formula (IV) are disclosed wherein [symbol] represents a double bond; Y is a covalent bond; Z is a covalent bond; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is a covalent bond; Z is a covalent bond; R₂ is hydrogen; R₃ and R₅ are absent; R₄ is heterocycle wherein the heterocycle is selected from imidazolyl, isoxazolyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (IV) are disclosed wherein [symbol] represents a double bond; Y is a covalent bond; Z is a covalent bond; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (IV) are disclosed wherein [symbol] represents a double bond; Y is a covalent bond; Z is a covalent bond; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (IV) are disclosed wherein [symbol] represents a single bond; Y is a covalent bond; Z is a covalent bond; R₂, R₃ and R₅ are hydrogen; R₄ is heterocycle; and R₁ is as defined in formula (I).

In another embodiment of the present invention, compounds of formula (IV) are disclosed wherein [symbol] represents a single bond; Y is a covalent bond; Z is a covalent bond; R₂, R₃ and R₅ are hydrogen; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR OR₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a single bond; Y is a covalent bond; Z is a covalent bond; R₂, R₃ and R₅ are hydrogen; R₄ is heterocycle wherein the heterocycle is selected from imidazolyl, isoxazolyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (IV) are disclosed wherein [symbol] represents a single bond; Y is a covalent bond; Z is a covalent bond; R₂, R₃, and R₅ are hydrogen; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (IV) are disclosed wherein [symbol] represents a single bond; Y is a covalent bond; Z is a covalent; R₂, R₃, and R₅ are hydrogen; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (IV) are disclosed wherein Y is a covalent bond; Z is CH₂; and R₁, R₂, R₃, R₄ and R₅ are as defined in formula (I).

In another embodiment of the present invention, compounds of formula (IV) are disclosed wherein [symbol] represents a double bond; Y is a covalent bond; Z is CH₂; R₂ is hydrogen; R₃ and R₅ are absent; R₄ is heterocycle; and R₁ is as defined in formula (I).

In another embodiment of the present invention, compounds of formula (IV) are disclosed wherein [symbol] represents a double bond; Y is a covalent bond; Z is CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is a covalent bond; Z is CH₂; R₂ is hydrogen; R₃ and R₅ are absent; R₄ is heterocycle wherein the heterocycle is selected from imidazolyl, isoxazolyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (IV) are disclosed wherein [symbol] represents a double bond; Y is a covalent bond; Z is CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (IV) are disclosed wherein [symbol] represents a double bond; Y is a covalent bond; Z is CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (IV) are disclosed wherein [symbol] represents a single bond; Y is a covalent bond; Z is CH₂; R₂ and R₃ are hydrogen; R₅ is selected from hydrogen or hydroxy; R₄ is heterocycle; and R₁ is as defined in formula (I).

In another embodiment of the present invention, compounds of formula (IV) are disclosed wherein [symbol] represents a single bond; Y is a covalent bond; Z is CH₂; R₂ and R₃ are hydrogen; R₅ is selected from hydrogen or hydroxy; R₄ is selected from heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a single bond; Y is a covalent bond; Z is CH₂; R₂ and R₃ are hydrogen; R₅ is selected from hydrogen or hydroxy; R₄ is selected from heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (IV) are disclosed wherein [symbol] represents a single bond; Y is a covalent bond; Z is CH₂; R₂ and R₃ are hydrogen; R₅ is selected from hydrogen or hydroxy; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (IV) are disclosed wherein [symbol] represents a single bond; Y is a covalent bond; Z is CH₂; R₂ and R₃ are hydrogen; R₅ is selected from hydrogen or hydroxy; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (IV) are disclosed wherein [symbol] represents a double bond; Y is a covalent bond; Z is CH₂; R₂ is heterocycle; R₃ and R₅ are absent; R₄ is hydrogen; and R₁ is as defined in formula (I).

In another embodiment of the present invention, compounds of formula (IV) are disclosed wherein [symbol] represents a double bond; Y is a covalent bond; Z is CH₂; R₂ is selected from heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is a covalent bond; Z is CH₂; R₂ is selected from heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NE)NR₁₀R₁₁; R₃ and R₅ are absent; R₄ is hydrogen; and R₁, R₁₀, and R₁₁ are as defined in formula (I).

In another embodiment of the present invention, compounds of formula (IV) are disclosed wherein [symbol] represents a double bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (III) are disclosed wherein [symbol] represents a double bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (IV) are disclosed wherein [symbol] represents a single bond; Y is a covalent bond; Z is CH₂; R₂ is heterocycle; R₃ is selected from hydrogen or hydroxy; R₄ and R₅ are hydrogen; and R₁ is as defined in formula (I).

In another embodiment of the present invention, compounds of formula (IV) are disclosed wherein [symbol] represents a single bond; Y is a covalent bond; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ is selected from hydrogen or hydroxy; R₄ and R₅ are hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a single bond; Y is a covalent bond; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ is selected from hydrogen or hydroxy; R₄ and R₅ are hydrogen; and R₁, R₁₀, and R₁₁ are as defined in formula (I).

In another embodiment of the present invention, compounds of formula (IV) are disclosed wherein [symbol] represents a single bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ is selected from hydrogen or hydroxy; R₄ and R₅ are hydrogen; and R₁ is as defined in formula (I).

In another embodiment of the present invention, compounds of formula (III) are disclosed wherein [symbol] represents a single bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ is selected from hydrogen or hydroxy; R₄ and R₅ are hydrogen; and R₁ is as defined in formula (I).

In another embodiment of the present invention, compounds of formula (IV) are disclosed wherein Y is a covalent bond; Z is CH₂CH₂; and R₁, R₂, R₃, R₄, and R₅ are as defined in formula (I).

In another embodiment of the present invention, compounds of formula (IV) are disclosed wherein [symbol] represents a double bond; Y is a covalent bond; Z is CH₂CH₂; R₂ is hydrogen; R₃ and R₅ are absent; R₄ is heterocycle; and R₁ is as defined in formula (I).

In another embodiment of the present invention, compounds of formula (IV) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁ and R₁, R₁₀, and R₁₁ are as defined in formula (I).

In another embodiment of the present invention, compounds of formula (IV) are disclosed wherein [symbol] represents a double bond; Y is a covalent bond; Z is CH₂CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (IV) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (IV) are disclosed wherein [symbol] represents a double bond; Y is a covalent bond; Z is CH₂CH₂; R₂ is heterocycle; R₃ and R₅ are absent; R₄ is hydrogen; and R₁ is as defined in formula (I).

In another embodiment of the present invention, compounds of formula (IV) are disclosed wherein [symbol] represents a double bond; Y is a covalent bond; Z is CH₂CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is a covalent bond; Z is CH₂CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (IV) are disclosed wherein [symbol] represents a double bond; Y is a covalent bond; Z is CH₂CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (IV) are disclosed wherein [symbol] represents a double bond; Y is a covalent bond; Z is CH₂CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (IV) are disclosed wherein Y is CH₂; Z is CH₂; and R₁, R₂, R₃, R₄, and R₅ are as defined in formula (I).

In another embodiment of the present invention, compounds of formula (IV) are disclosed wherein [symbol] represents a double bond; Y is CH₂; Z is CH₂; R₂ is heterocycle; R₃ and R₅ are absent; R₄ is hydrogen; and R₁ is as defined in formula (I).

In another embodiment of the present invention, compounds of formula (IV) are disclosed wherein [symbol] represents a double bond; Y is CH₂; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (IV) are disclosed wherein [symbol] represents a double bond; Y is CH₂; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (IV) are disclosed wherein [symbol] represents a double bond; Y is CH₂; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (IV) are disclosed wherein [symbol] represents a double bond; Y is CH₂; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

Representative compounds of formula (IV) include, but are not limited to:

(cis)-5-(6-chloro-3-pyridinyl)-1,2,3,3a,4,6a-hexahydrocyclopenta[c]pyrrole;

(cis)-5-(5-methoxy-3-pyridinyl)-1,2,3,3a,4,6a-hexahydrocyclopenta[c]pyrrole;

(cis)-5-(6-chloro-5-methyl-3-pyridinyl)-1,2,3,3a,4,6a-hexahydrocyclopenta[c]pyrrole;

(cis)-5-(3-pyridinyl)-1,2,3,3a,4,6a-hexahydrocyclopenta[c]pyrrole;

(cis)-5-(3-methyl-5-isoxazolyl)-1,2,3,3a,4,6a-hexahydrocyclopenta[c]pyrrole;

(cis)-5-(5,6-dichloro-3-pyridinyl)-1,2,3,3a,4,6a-hexahydrocyclopenta[c]pyrrole;

(cis)-2-methyl-5-(3-methyl-5-isoxazolyl)-1,2,3,3a,4,6a-hexahydrocyclopenta[c]pyrrole;

(cis)-5-(5-chloro-3-pyridinyl)-1,2,3,3a,4,6a-hexahydrocyclopenta[c]pyrrole;

(cis)-5-(3-bromo-1,2,4-thiadiazol-5-yl)-1,2,3,3a,4,6a-hexahydrocyclopenta[c]pyrrole;

(cis)-5-(1,3-thiazol-2-yl)-1,2,3,3a,4,6a-hexahydrocyclopenta[c]pyrrole;

(cis)-5-(3,5-dimethyl-4-isoxazolyl)-1,2,3,3a,4,6 a-hexahydrocyclopenta[c]pyrrole;

(cis)-5-(1H-imidazol-4-yl)-1,2,3,3a,4,6a-hexahydrocyclopenta[c]pyrrole;

(cis)-5-(1,3-thiazol-5-yl)-1,2,3,3a,4,6a-hexahydrocyclopenta[c]pyrrole;

(cis)-5-(imidazo[1,2-a]pyridin-3-yl)-1,2,3,3a,4,6a-hexahydrocyclopenta[c]pyrrole;

(cis)-5-(imidazo[1,2-a]pyridin-6-yl)-1,2,3,3a,4,6a-hexahydrocyclopenta[c]pyrrole;

(cis)-5-(thieno[3,2-b]pyridin-2-yl)-1,2,3,3a,4,6a-hexahydrocyclopenta[c]pyrrole;

(cis)-5-(1,2,3,3a,4,6a-hexahydrocyclopenta[c]pyrrol-5-yl)-2-thiophenesulfonamide;

(cis)-5-(6-methyl-3-pyridinyl)-1,2,3,3a,4,6a-hexahydrocyclopenta[c]pyrrole;

(cis)-5-(2-methyl-3-pyridinyl)-1,2,3,3a,4,6a-hexahydrocyclopenta[c]pyrrole;

(cis)-5-(6-chloro-5-fluoro-3-pyridinyl)-1,2,3,3a,4,6a-hexahydrocyclopenta[c]pyrrole;

(cis)-5-(6-fluoro-3-pyridinyl)-1,2,3,3a,4,6a-hexahydrocyclopenta[c]pyrrole;

(cis)-5-(1,2,3,3a,4,6a-hexahydrocyclopenta[c]pyrrol-5-yl)-2-thiophenecarboximidamide;

(cis)-5-(2-methyl-2H-tetrazol-5-yl)-1,2,3,3a,4,6a-hexahydrocyclopenta[c]pyrrole;

(cis)-5-(thieno[2,3-b]pyridin-5-yl)-1,2,3,3a,4,6a-hexahydrocyclopenta[c]pyrrole;

(cis)-5-(imidazo[1,2-a]pyridin-7-yl)-1,2,3,3a,4,6a-hexahydrocyclopenta[c]pyrrole;

(cis)-5-(2-pyridinyl)-1,2,3,3a,4,6a-hexahydrocyclopenta[c]pyrrole;

(cis)-5-(4-pyridinyl)-1,2,3,3a,4,6a-hexahydrocyclopenta[c]pyrrole;

(cis)-5-(5-nitro-1,3-thiazol-2-yl)-1,2,3,3a,4,6a-hexahydrocyclopenta[c]pyrrole;

(cis)-5-(6-methyl-2-pyridinyl)-1,2,3,3a,4,6a-hexahydrocyclopenta[c]pyrrole;

(cis)-5-(1,3,4-thiadiazol-2-yl)-1,2,3,3a,4,6a-hexahydrocyclopenta[c]pyrrole;

(endo)-5-(3-pyridinyl)octahydrocyclopenta[c]pyrrole;

(cis)-5-(6-chloro-3-pyridinyl)octahydrocyclopenta[c]pyrrol-5-ol;

(endo)-5-(6-chloro-3-pyridinyl)octahydrocyclopenta[c]pyrrole;

(exo)-5-(6-chloro-3-pyridinyl)octahydrocyclopenta[c]pyrrole;

(cis)-5-(5-bromo-3-pyridinyl)-1,2,3,3a,4,6a-hexahydrocyclopenta[c]pyrrole;

(cis)-5-(5-vinyl-3-pyridinyl)-1,2,3,3a,4,6a-hexahydrocyclopenta[c]pyrrole;

(cis)-5-(1,2,3,3a,4,6a-hexahydrocyclopenta[c]pyrrol-5-yl)nicotinonitrile;

(cis)-5-(6-chloro-3-pyridinyl)-1,2,3,3a,4,6a-hexahydrocyclopenta[c]pyrrole;

(cis)-5-(6-chloro-3-pyridinyl)-1,2,3,3a,4,6a-hexahydrocyclopenta[c]pyrrole;

(cis)-6-(6-chloro-3-pyridinyl)-1,2,3,3a,4,6a-hexahydrocyclopenta[c]pyrrole;

(cis)-4-(3-pyridinyl)octahydrocyclopenta[c]pyrrol-4-ol;

(endo)-4-(6-chloro-3-pyridinyl)octahydrocyclopenta[c]pyrrole;

(cis)-6-(6-methyl-3-pyridinyl)-3-azabicyclo[3.2.0]hept-6-ene;

(endo)-6-(6-chloro-3-pyridinyl)-3-azabicyclo[3.2.0]heptane;

(exo)-6-(6-chloro-3-pyridinyl)-3-azabicyclo[3.2.0]heptane;

(cis)-6-(5,6-dichloro-3-pyridinyl)-3-azabicyclo[3.2.0]hept-6-ene;

(endo)-6-(5,6-dichloro-3-pyridinyl)-3-azabicyclo[3.2.0]heptane;

(cis)-5-(6-bromo-3-pyridinyl)-1,2,3,3a,4,6a-hexahydrocyclopenta[c]pyrrole;

(cis)-5-(5-methyl-3-pyridinyl)-1,2,3,3a,4,6a-hexahydrocyclopenta[c]pyrrole;

(cis)-5-(6-chloro-5-methoxy-3-pyridinyl)-1,2,3,3a,4,6a-hexahydrocyclopenta[c]pyrrole;

(cis)-5-(6-bromo-5-methoxy-3-pyridinyl)-1,2,3,3a,4,6a-hexahydrocyclopenta[c]pyrrole;

(cis)-5-(5-methoxy-6-methyl-3-pyridinyl)-1,2,3,3a,4,6a-hexahydrocyclopenta[c]pyrrole;

(cis)-2-chloro-5-(1,2,3,3a,4,6a-hexahydrocyclopenta[c]pyrrol-5-yl)nicotinonitrile;

(cis)-2-bromo-5-(1,2,3,3a,4,6a-hexahydrocyclopenta[c]pyrrol-5-yl)nicotinonitrile;

(cis)-5-(1,2,3,3a,4,6a-hexahydrocyclopenta[c]pyrrol-5-yl)-2-methylnicotinonitrile;

(cis)-5-(5-bromo-6-chloro-3-pyridinyl)-1,2,3,3a,4,6a-hexahydrocyclopenta[c]pyrrole;

(cis)-5-(5,6-dibromo-3-pyridinyl)-1,2,3,3a,4,6a-hexahydrocyclopenta[c]pyrrole;

(cis)-5-(6-bromo-5-chloro-3-pyridinyl)-1,2,3,3a,4,6a-hexahydrocyclopenta[c]pyrrole;

(cis)-5-(5-chloro-6-methyl-3-pyridinyl)-1,2,3,3a,4,6a-hexahydrocyclopenta[c]pyrrole;

(cis)-5-(5-bromo-6-methyl-3-pyridinyl)-1,2,3,3a,4,6a-hexahydrocyclopenta[c]pyrrole;

(cis)-5-(6-bromo-5-methyl-3-pyridinyl)-1,2,3,3a,4,6a-hexahydrocyclopenta[c]pyrrole;

(cis)-2-(1,2,3,3a,4,6a-hexahydrocyclopenta[c]pyrrol-5-yl)furo[3,2-b]pyridine;

(cis)-7-(3-pyridinyl)-2,3,3a,4,5,7a-hexahydro-1H-isoindole;

(cis)-7-(6-chloro-3-pyridinyl)-2,3,3a,4,5,7a-hexahydro-1H-isoindole;

(cis)-7-(6-bromo-3-pyridinyl)-2,3,3a,4,5,7a-hexahydro-1H-isoindole;

(cis)-7-(6-fluoro-3-pyridinyl)-2,3,3a,4,5,7a-hexahydro-1H-isoindole;

(cis)-7-(6-methyl-3-pyridinyl)-2,3,3a,4,5,7a-hexahydro-1H-isoindole;

(cis)-5-(2,3,3a,6,7,7a-hexahydro-1H-isoindol-4-yl)nicotinonitrile;

(cis)-7-(5-methyl-3-pyridinyl)-2,3,3a,4,5,7a-hexahydro-1H-isoindole;

(cis)-7-(5-chloro-3-pyridinyl)-2,3,3a,4,5,7a-hexahydro-1H-isoindole;

(cis)-7-(5-bromo-3-pyridinyl)-2,3,3a,4,5,7a-hexahydro-1H-isoindole;

(cis)-7-(5-fluoro-3-pyridinyl)-2,3,3a,4,5,7a-hexahydro-1H-isoindole;

(cis)-7-(5-vinyl-3-pyridinyl)-2,3,3a,4,5,7a-hexahydro-1H-isoindole;

(cis)-7-(5-methoxy-3-pyridinyl)-2,3,3a,4,5,7a-hexahydro-1H-isoindole;

(cis)-7-(5-vinyloxy-3-pyridinyl)-2,3,3a,4,5,7a-hexahydro-1H-isoindole;

(cis)-7-(5-ethynyl-3-pyridinyl)-2,3,3a,4,5,7a-hexahydro-1H-isoindole;

(cis)-7-(5,6-dichloro-3-pyridinyl)-2,3,3a,4,5,7a-hexahydro-1H-isoindole;

(cis)-7-(6-bromo-5-chloro-3-pyridinyl)-2,3,3a,4,5,7a-hexahydro-1H-isoindole;

(cis)-5-(2,3,3a,6,7,7a-hexahydro-1H-isoindol-4-yl)-2-methylnicotinonitrile;

(cis)-2-chloro-5-(2,3,3a,6,7,7a-hexahydro-1H-isoindol-4-yl)nicotinonitrile;

(cis)-2-bromo-5-(2,3,3a,6,7,7a-hexahydro-1H-isoindol-4-yl)nicotinonitrile;

(cis)-7-(6-chloro-5-methyl-3-pyridinyl)-2,3,3a,4,5,7a-hexahydro-1H-isoindole;

(cis)-7-(6-bromo-5-methyl-3-pyridinyl)-2,3,3a,4,5,7a-hexahydro-1H-isoindole;

(cis)-7-(5-methoxy-6-methyl-3-pyridinyl)-2,3,3a,4,5,7a-hexahydro-1H-isoindole;

(cis)-7-(6-chloro-5-methoxy-3-pyridinyl)-2,3,3a,4, 5,7a-hexahydro-1H-isoindole;

(cis)-7-(6-bromo-5-methoxy-3-pyridinyl)-2,3,3a,4,5,7a-hexahydro-1H-isoindole;

(cis)-7-(3-methyl-5-isoxazolyl)-2,3,3a,4,5,7a-hexahydro-1H-isoindole;

(cis)-2-(2,3,3a,6,7,7a-hexahydro-1H-isoindol-4-yl)furo[3,2-b]pyridine;

(cis)-6-(3-pyridinyl)-1,2,3,3a,4,6a-hexahydrocyclopenta[c]pyrrole;

(cis)-6-(6-chloro-3-pyridinyl)-1,2,3,3a,4,6a-hexahydrocyclopenta[c]pyrrole;

(cis)-6-(6-bromo-3-pyridinyl)-1,2,3,3a,4,6a-hexahydrocyclopenta[c]pyrrole;

(cis)-6-(6-methyl-3-pyridinyl)-1,2,3,3a,4,6a-hexahydrocyclopenta[c]pyrrole;

(cis)-6-(6-fluoro-3-pyridinyl)-1,2,3,3a,4,6a-hexahydrocyclopenta[c]pyrrole;

(cis)-5-(o 1,2,3,3a,6,6a-hexahydrocyclopenta[c]pyrrol-4-yl)nicotinonitrile;

(cis)-6-(5-methyl-3-pyridinyl)-1,2,3,3a,4,6a-hexahydrocyclopenta[c]pyrrole;

(cis)-6-(5-chloro-3-pyridinyl)-1,2,3,3a,4,6a-hexahydrocyclopenta[c]pyrrole;

(cis)-6-(5-fluoro-3-pyridinyl)-1,2,3,3a,4,6a-hexahydrocyclopenta[c]pyrrole;

(cis)-6-(5-bromo-3-pyridinyl)-1,2,3,3a,4,6a-hexahydrocyclopenta[c]pyrrole;

(cis)-6-(5-vinyl-3-pyridinyl)-1,2,3,3a,4,6a-hexahydrocyclopenta[c]pyrrole;

(cis)-6-(5-methoxy-3-pyridinyl)-1,2,3,,3a,4,6a-hexahydrocyclopenta[c]pyrrole;

(cis)-6-(5-vinyloxy-3-pyridinyl)-1,2,3,3a,4,6a-hexahydrocyclopenta[c]pyrrole;

(cis)-6-(5-ethynyl-3-pyridinyl)-1,2,3,3a,4,6a-hexahydrocyclopenta[c]pyrrole;

(cis)-6-(5,6-dichloro-3-pyridinyl)-1,2,3,3a,4,6a-hexahydrocyclopenta[c]pyrrole;

(cis)-6-(6-bromo-3-pyridinyl)-1,2,3,3a,4,6a-hexahydrocyclopenta[c]pyrrole;

(cis)-5-(1,2,3,3a,6,6a-hexahydrocyclopenta[c]pyrrol-4-yl)-2-methylnicotinonitrile;

(cis)-2-chloro-5-(,2,3,3a,6,6a-hexahydrocyclopenta[c]pyrrol-4-yl)nicotinonitrile;

(cis)-2-bromo-5-(1,2,3,3a,6,6a-hexahydrocyclopenta[c]pyrrol-4-yl)nicotinonitrile;

(cis)-6-(6-chloro-5-methyl-3-pyridinyl)-1,2,3,3a,4,6a-hexahydrocyclopenta[c]pyrrole;

(cis)-6-(6-bromo-5-methyl-3-pyridinyl)-1,2,3,3a,4,6a-hexahydrocyclopenta[c]pyrrole;

(cis)-6-(5-methoxy-6-methyl-3-pyridinyl)-1,2,3,3a,4,6a-hexahydrocyclopenta[c]pyrrole;

(cis)-6-(6-chloro-5-methoxy-3-pyridinyl)-1,2,3,3a,4,6a-hexahydrocyclopenta[c]pyrrole;

(cis)-6-(6-bromo-5-methoxy-3-pyridinyl)-1,2,3,3a,4,6a-hexahydrocyclopenta[c]pyrrole;

(cis)-6-(3-methyl-5-isoxazolyl)-1,2,3,3a,4,6a-hexahydrocyclopenta[c]pyrrole;

(cis)-2-(1,2,3,3a,6,6a-hexahydrocyclopenta[c]pyrrol-4-yl)furo[3,2-b]pyridine;

(cis)-6-(3-pyridinyl)-2,3,3a,4,5,7a-hexahydro-1H-isoindole;

(cis)-6-(6-methyl-3-pyridinyl)-2,3,3a,4,5,7a-hexahydro-1H-isoindole;

(cis)-6-(6-bromo-3-pyridinyl)-2,3,3a,4,5,7a-hexahydro-1H-isoindole;

(cis)-6-(6-chloro-3-pyridinyl)-2,3,3a,4,5,7a-hexahydro-1H-isoindole;

(cis)-6-(6-fluoro-3-pyridinyl)-2,3,3a,4,5,7a-hexahydro-1H-isoindole;

(cis)-5-(2,3,3a,6,7,7a-hexahydro-1H-isoindol-5-yl)nicotinonitrile;

(cis)-6-(5-methyl-3-pyridinyl)-2,3,3a,4,5,7a-hexahydro-1H-isoindole;

(cis)-6-(5-chloro-3-pyridinyl)-2,3,3a,4,5,7a-hexahydro-1H-isoindole;

(cis)-6-(5-fluoro-3-pyridinyl)-2,3,3a,4,5,7a-hexahydro-1H-isoindole;

(cis)-6-(5-bromo-3-pyridinyl)-2,3,3a,4,5,7a-hexahydro-1H-isoindole;

(cis)-6-(5-vinyl-3-pyridinyl)-2,3,3a,4,5,7a-hexahydro-1H-isoindole;

(cis)-6-(5-methoxy-3-pyridinyl)-2,3,3a,4,5,7a-hexahydro-1H-isoindole;

(cis)-6-(5-vinyloxy-3-pyridinyl)-2,3,3a,4,5,7a-hexahydro-1H-isoindole;

(cis)-6-(5-ethynyl-3-pyridinyl)-2,3,3a,4,5,7a-hexahydro-1H-isoindole;

(cis)-6-(5,6-dichloro-3-pyridinyl)-2,3,3a,4,5,7a-hexahydro-1H-isoindole;

(cis)-6-(6-bromo-5-chloro-3-pyridinyl)-2,3,3a,4,5,7a-hexahydro-1H-isoindole;

(cis)-5-(2,3,3a,6,7,7a-hexahydro-1H-isoindol-5-yl)-2-methylnicotinonitrile;

(cis)-2-chloro-5-(2,3,3a,6,7,7a-hexahydro-1H-isoindol-5-yl)nicotinonitrile;

(cis)-2-bromo-5-(2,3,3a,6,7,7a-hexahydro-1H-isoindol-5-yl)nicotinonitrile;

(cis)-6-(6-chloro-5-methyl-3-pyridinyl)-2,3,3a,4,5,7a-hexahydro-1H-isoindole;

(cis)-6-(6-bromo-5-methyl-3-pyridinyl)-2,3,3a,4,5,7a-hexahydro-1H-isoindole;

(cis)-6-(5-methoxy-6-methyl-3-pyridinyl)-2,3,3a,4,5,7a-hexahydro-1H-isoindole;

(cis)-6-(6-chloro-5-methoxy-3-pyridinyl)-2,3,3a,4,5,7a-hexahydro-1H-isoindole;

(cis)-6-(6-bromo-5-methoxy-3-pyridinyl)-2,3,3a,4,5,7a-hexahydro-1H-isoindole;

(cis)-6-(3-methyl-5-isoxazolyl)-2,3,3a,4,5,7a-hexahydro-1H-isoindole;

(cis)-2-(2,3,3a,6,7,7a-hexahydro-1H-isoindol-5-yl)furo[3,2-b]pyridine;

(cis)-5-(3-pyridinyl)-2,3,3a,4,7,7a-hexahydro-1H-isoindole;

(cis)-5-(6-methyl-3-pyridinyl)-2,3,3a,4,7,7a-hexahydro-1H-isoindole;

(cis)-5-(6-chloro-3-pyridinyl)-2,3,3a,4,7,7a-hexahydro-1H-isoindole;

(cis)-5-(6-bromo-3-pyridinyl)-2,3,3a,4,7,7a-hexahydro-1H-isoindole;

(cis)-5-(6-fluoro-3-pyridinyl)-2,3,3a,4,7,7a-hexahydro-1H-isoindole;

(cis)-5-(2,3,3a,4,7,7a-hexahydro-1H-isoindol-5-yl)nicotinonitrile;

(cis)-5-(5-methyl-3-pyridinyl)-2,3,3a,4,7,7a-hexahydro-1H-isoindole;

(cis)-5-(5-chloro-3-pyridinyl)-2,3,3a,4,7,7a-hexahydro-1H-isoindole;

(cis)-5-(5-fluoro-3-pyridinyl)-2,3,3a,4,7,7a-hexahydro-1H-isoindole;

(cis)-5-(5-bromo-3-pyridinyl)-2,3,3a,4,7,7a-hexahydro-1H-isoindole;

(cis)-5-(5-vinyl-3-pyridinyl)-2,3,3a,4,7,7a-hexahydro-1H-isoindole;

(cis)-5-(5-methoxy-3-pyridinyl)-2,3,3a,4,7,7a-hexahydro-1H-isoindole;

(cis)-5-(5-vinyloxy-3-pyridinyl)-2,3,3a,4,7,7a-hexahydro-1H-isoindole;

(cis)-5-(5-ethynyl-3-pyridinyl)-2,3,3a,4,7,7a-hexahydro-1H-isoindole;

(cis)-5-(5,6-dichloro-3-pyridinyl)-2,3,3a,4,7,7a-hexahydro-1H-isoindole;

(cis)-5-(6-bromo-5-chloro-3-pyridinyl)-2,3,3a,4,7,7a-hexahydro-1H-isoindole;

(cis)-5-(2,3,3a,4,7,7a-hexahydro-1H-isoindol-5-yl)-2-methylnicotinonitrile;

(cis)-2-chloro-5-(2,3,3a,4,7,7a-hexahydro-1H-isoindol-5-yl)nicotinonitrile;

(cis)-2-bromo-5-(2,3,3a,4,7,7a-hexahydro-1H-isoindol-5-yl)nicotinonitrile;

(cis)-5-(6-chloro-5-methyl-3-pyridinyl)-2,3,3a,4,7,7a-hexahydro-1H-isoindole;

(cis)-5-(6-bromo-5-methyl-3-pyridinyl)-2,3,3a,4,7,7a-hexahydro-1H-isoindole;

(cis)-5-(5-methoxy-6-methyl-3-pyridinyl)-2,3,3a,4,7,7a-hexahydro-1H-isoindole;

(cis)-5-(6-chloro-5-methoxy-3-pyridinyl)-2,3,3a,4,7,7a-hexahydro-1H-isoindole;

(cis)-5-(6-bromo-5-methoxy-3-pyridinyl)-2,3,3a,4,7,7a-hexahydro-1H-isoindole;

(cis)-2-(2,3,3a,4,7,7a-hexahydro-1H-isoindol-5-yl)furo[3,2-b]pyridine;

(cis)-5-(3-methyl-5-isoxazolyl)-2,3,3a,4,7,7a-hexahydro-1H-isoindole;

(cis)-6-(3-pyridinyl)-3-azabicyclo[3.2.0]hept-6-ene;

(cis)-6-(6-bromo-3-pyridinyl)-3-azabicyclo[3.2.0]hept-6-ene;

(cis)-6-(6-fluoro-3-pyridinyl)-3-azabicyclo[3.2.0]hept-6-ene;

(cis)-6-(6-methyl-3-pyridinyl)-3-azabicyclo[3.2.0]hept-6-ene;

(cis)-5-(3-azabicyclo[3.2.0]hept-6-en-6-yl)nicotinonitrile;

(cis)-6-(5-chloro-3-pyridinyl)-3-azabicyclo[3.2.0]hept-6-ene;

(cis)-6-(5-bromo-3-pyridinyl)-3-azabicyclo[3.2.0]hept-6-ene;

(cis)-6-(5-fluoro-3-pyridinyl)-3-azabicyclo[3.2.0]hept-6-ene;

(cis)-6-(5-methyl-3-pyridinyl)-3-azabicyclo[3.2.0]hept-6-ene;

(cis)-6-(5-vinyl-3-pyridinyl)-3-azabicyclo[3.2.0]hept-6-ene;

(cis)-6-(5-methoxy-3-pyridinyl)-3-azabicyclo[3.2.0]hept-6-ene;

(cis)-6-(5-vinyloxy-3-pyridinyl)-3-azabicyclo[3.2.0]hept-6-ene;

(cis)-6-(5-ethynyl-3-pyridinyl)-3-azabicyclo[3.2.0]hept-6-ene;

(cis)-6-(6-bromo-5-chloro-3-pyridinyl)-3-azabicyclo[3.2.0]hept-6-ene;

(cis)-5-(3-azabicyclo[3.2.0]hept-6-en-6-yl)-2-methylnicotinonitrile;

(cis)-5-(3-azabicyclo[3.2.0]hept-6-en-6-yl)-2-bromonicotinonitrile;

(cis)-5-(3-azabicyclo[3.2.0]hept-6-en-6-yl)-2-chloronicotinonitrile;

(cis)-6-(6-chloro-5-methyl-3-pyridinyl)-3-azabicyclo[3.2.0]hept-6-ene;

(cis)-6-(6-bromo-5-methyl-3-pyridinyl)-3-azabicyclo[3.2.0]hept-6-ene;

(cis)-6-(5-methoxy-6-methyl-3-pyridinyl)-3-azabicyclo[3.2.0]hept-6-ene;

(cis)-6-(6-bromo-5-methoxy-3-pyridinyl)-3-azabicyclo[3.2.0]hept-6-ene;

(cis)-6-(6-chloro-5-methoxy-3-pyridinyl)-3-azabicyclo[3.2.0]hept-6-ene; or a

pharmaceutically acceptable salt, amide, ester or prodrugs thereof.

In another embodiment of the present invention, compounds of formula (V) are disclosed

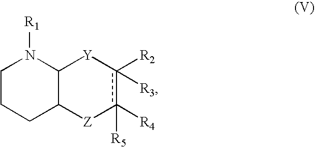

or a pharmaceutically acceptable salt, amide, ester or prodrug thereof wherein Y, Z, R₁, R₂, R₃, R₄, and R₅ are as defined in formula (I).

In another embodiment of the present invention, compounds of formula (V) are disclosed wherein Y is a covalent bond; Z is a covalent bond; and R₁, R₂, R₃, R₄, and R₅ are as defined in formula (I).

In another embodiment of the present invention, compounds of formula (V) are disclosed wherein Y is a covalent bond; Z is CH₂; and R₁, R₂, R₃, R₄, and R₅ are as defined in formula (I).

In another embodiment of the present invention, compounds of formula (V) are disclosed wherein [symbol] represents a double bond; Y is a covalent bond; Z is CH₂; R₂ is hydrogen; R₃ and R₅ are absent; R₄ is heterocycle; and R₁ is as defined in formula (I).

In another embodiment of the present invention, compounds of formula (V) are disclosed wherein [symbol] represents a double bond; Y is a covalent bond; Z is CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (V) are disclosed wherein [symbol] represents a double bond; Y is a covalent bond; Z is CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (V) are disclosed wherein [symbol] represents a double bond; Y is a covalent bond; Z is CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (V) are disclosed wherein [symbol] represents a double bond; Y is a covalent bond; Z is CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (V) are disclosed wherein [symbol] represents a double bond; Y is a covalent bond; Z is CH₂; R₂ is heterocycle; R₃ and R₅ are absent; R₄ is hydrogen; and R₁ is as defined in formula (I).

In another embodiment of the present invention, compounds of formula (V) are disclosed wherein [symbol] represents a double bond; Y is a covalent bond; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (V) are disclosed wherein [symbol] represents a double bond; Y is a covalent bond; Z is CH₂; R₂ is heterocycle wherein the heterocycle is selected from imdazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (V) are disclosed wherein [symbol] represents a double bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (V) are disclosed wherein [symbol] represents a double bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (V) are disclosed wherein Y is CH₂; Z is a covalent bond; and R₁, R₂, R₃, R₄, and R₅ are as defined in formula (I).

In another embodiment of the present invention, compounds of formula (V) are disclosed wherein [symbol] represents a double bond; Y is CH₂; Z is a covalent bond; R₂ is heterocycle; R₃ and R₅ are absent; R₄ is hydrogen; and R₁ is as defined in formula (I).

In another embodiment of the present invention, compounds of formula (V) are disclosed wherein [symbol] represents a double bond; Y is CH₂; Z is a covalent bond; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (V) are disclosed wherein [symbol] represents a double bond; Y is CH₂; Z is a covalent bond; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R_{1, 1})sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (V) are disclosed wherein [symbol] represents a double bond; Y is CH₂; Z is a covalent bond; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (V) are disclosed wherein [symbol] represents a double bond; Y is CH₂; Z is a covalent bond; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (V) are disclosed wherein Y is a covalent bond; Z is CH₂CH₂; and R₁, R₂, R₃, R₄, and R₅ are as defined in formula (I).

In another embodiment of the present invention, compounds of formula (V) are disclosed wherein [symbol] represents a double bond; Y is a covalent bond; Z is CH₂CH₂; R₂ is hydrogen; R₃ and R₅ are absent; R₄ is heterocycle; and R₁ is as defined in formula (I).

In another embodiment of the present invention, compounds of formula (V) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (V) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (V) are disclosed wherein [symbol] represents a double bond; Y is a covalent bond; Z is CH₂CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (V) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (V) are disclosed wherein [symbol] represents a double bond; Y is a covalent bond; Z is CH₂CH₂; R₂ is heterocycle; R₃ and R₅ are absent; R₄ is hydrogen; and R₁ is as defined in formula (I).

In another embodiment of the present invention, compounds of formula (V) are disclosed wherein [symbol] represents a double bond; Y is a covalent bond; Z is CH₂CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In anther embodiment of the present invention, compounds of formula (V) are disclosed wherein [symbol] represents a double bond; Y is a covalent bond; Z is CH₂CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (V) are disclosed wherein [symbol] represents a double bond; Y is a covalent bond; Z is CH₂CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (V) are disclosed wherein [symbol] represents a double bond; Y is a covalent bond; Z is CH₂CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (V) are disclosed wherein Y is CH₂; Z is CH₂; and R₁, R₂, R₃, R₄, and R₅ are as defined in formula (I).

In another embodiment of the present invention, compounds of formula (V) are disclosed wherein [symbol] represents a double bond; Y is CH₂; Z is CH₂; R₂ is heterocycle; R₃ and R₅ are absent; R₄ is hydrogen; and R₁ is as defined in formula (I).

In another embodiment of the present invention, compounds of formula (V) are disclosed wherein [symbol] represents a double bond; Y is CH₂; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (V) are disclosed wherein [symbol] represents a double bond; Y is CH₂; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (V) are disclosed wherein [symbol] represents a double bond; Y is CH₂; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (V) are disclosed wherein [symbol] represents a double bond; Y is CH₂; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (V) are disclosed wherein Y is CH₂CH₂; Z is a covalent bond; and R₁, R₂, R₃, R₄, and R₅ are as defined in formula (I)

Representative compounds of formula (V) include, but are not limited to:

(cis)-8-(3-pyridinyl)-1,2,3,4,4a,5,6,8a-octahydroquinoline;

(cis)-8-(6-chloro-3-pyridinyl)-1,2,3,4,4a,5,6,8a-octahydroquinoline;

(cis)-8-(6-bromo-3-pyridinyl)-1,2,3,4,4a,5,6,8a-octahydroquinoline;

(cis)-8-(6-fluoro-3-pyridinyl)-1,2,3,4,4a,5,6,8a-octahydroquinoline;

(cis)-8-(6-methyl-3-pyridinyl)-1,2,3,4,4a,5,6,8a-octahydroquinoline;

(cis)-5-(1,2,3,4,4a,5,6,8a-octahydro-8-quinolinyl)nicotinonitrile;

(cis)-8-(5-methyl-3-pyridinyl)-1,2,3,4,4a,5,6,8a-octahydroquinoline;

(cis)-8-(5-chloro-3-pyridinyl)-1,2,3,4,4a,5,6,8a-octahydroquinoline;

(cis)-8-(5-fluoro-3-pyridinyl)-1,2,3,4,4a,5,6,8a-octahydroquinoline;

(cis)-8-(5-bromo-3-pyridinyl)-1,2,3,4,4a,5,6,8a-octahydroquinoline;

(cis)-8-(5-vinyl-3-pyridinyl)-1,2,3,4,4a,5,6,8a-octahydroquinoline;

(cis)-8-(5-methoxy-3-pyridinyl)-1,2,3,4,4a,5,6,8a-octahydroquinoline;

(cis)-8-(5-vinyloxy-3-pyridinyl)-1,2,3,4,4a,5,6,8a-octahydroquinoline;

(cis)-8-(5-ethynyl-3-pyridinyl)-1,2,3,4,4a,5,6,8a-octahydroquinoline;

(cis)-8-(5,6-dichloro-3-pyridinyl)-1,2,3,4,4a,5,6,8a-octahydroquinoline;

(cis)-8-(6-bromo-5-chloro-3-pyridinyl)-1,2,3,4,4a,5,6,8a-octahydroquinoline;

(cis)-2-methyl-5-(1,2,3,4,4a,5,6,8a-octahydro-8-quinolinyl)nicotinonitrile;

(cis)-2-bromo-5-(1,2,3,4,4a,5,6,8a-octahydro-8-quinolinyl)nicotinonitrile;

(cis)-2-chloro-5-(1,2,3,4,4a,5,6,8a-octahydro-8-quinolinyl)nicotinonitrile;

(cis)-8-(6-chloro-5-methyl-3-pyridinyl)-1,2,3,4,4a,5,6,8a-octahydroquinoline;

(cis)-8-(5-methoxy-6-methyl-3-pyridinyl)-1,2,3,4,4a,5,6,8a-octahydroquinoline;

(cis)-8-(6-chloro-5-methoxy-3-pyridinyl)-1,2,3,4,4a,5,6,8a-octahydroquinoline;

(cis)-8-(6-bromo-5-methoxy-3-pyridinyl)-1,2,3,4,4a,5,6,8a-octahydroquinoline;

(cis)-8-(3-methyl-5-isoxazolyl)-1,2,3,4,4a, 5,6,8a-octahydroquinoline;

(cis)-8-furo[3,2-b]pyridin-2-yl-1,2,3,4,4a,5,6,8a-octahydroquinoline;

(cis)-7-(3-pyridinyl)-2,3,4,4a,5,7a-hexahydro-1H-cyclopenta[b]pyridine;

(cis)-7-(6-chloro-3-pyridinyl)-2,3,4,4a,5,7a-hexahydro-1H-cyclopenta[b]pyridine;

(cis)-7-(6-bromo-3-pyridinyl)-2,3,4,4a,5,7a-hexahydro-1H-cyclopenta[b]pyridine;

(cis)-7-(6-fluoro-3-pyridinyl)-2,3,4,4a,5,7a-hexahydro-1H-cyclopenta[b]pyridine;

(cis)-7-(6-methyl-3-pyridinyl)-2,3,4,4a,5,7a-hexahydro-1H-cyclopenta[b]pyridine;

(cis)-5-(2,3,4,4a,5,7a-hexahydro-1H-cyclopenta[b]pyridin-7-yl)nicotinonitrile;

(cis)-7-(5-methyl-3-pyridinyl)-2,3,4,4a,5,7a-hexahydro-1H-cyclopenta[b]pyridine;

(cis)-7-(5-chloro-3-pyridinyl)-2,3,4,4a,5,7a-hexahydro-1H-cyclopenta[b]pyridine;

(cis)-7-(5-bromo-3-pyridinyl)-2,3,4,4a,5,7a-hexahydro-1H-cyclopenta[b]pyridine;

(cis)-7-(5-fluoro-3-pyridinyl)-2,3,4,4a,5,7a-hexahydro-1H-cyclopenta[b]pyridine;

(cis)-7-(5-vinyl-3-pyridinyl)-2,3,4,4a,5,7a-hexahydro-1H-cyclopenta[b]pyridine;

(cis)-7-(5-methoxy-3-pyridinyl)-2,3,4,4a,5,7a-hexahydro-1H-cyclopenta[b]pyridine;

(cis)-7-(5-vinyloxy-3-pyridinyl)-2,3,4,4a,5,7a-hexahydro-1H-cyclopenta[b]pyridine;

(cis)-7-(5-ethynyl-3-pyridinyl)-2,3,4,4a,5,7a-hexahydro-1H-cyclopenta[b]pyridine;

(cis)-7-(5,6-dichloro-3-pyridinyl)-2,3,4,4a,5,7a-hexahydro-1H-cyclopenta[b]pyridine;

(cis)-7-(6-bromo-5-chloro-3-pyridinyl)-2,3,4,4a,5,7a-hexahydro-1H-cyclopenta[b]pyridine;

(cis)-5-(2,3,4,4a,5,7a-hexahydro-1H-cyclopenta[b]pyridin-7-yl)-2-methylnicotinonitrile;

(cis)-2-bromo-5-(2,3,4,4a,5,7a-hexahydro-1H-cyclopenta[b]pyridin-7-yl)nicotinonitrile;

(cis)-2-chloro-5-(2,3,4,4a,5,7a-hexahydro-1H-cyclopenta[b]pyridin-7-yl)nicotinonitrile;

(cis)-7-(6-chloro-5-methyl-3-pyridinyl)-2,3,4,4a,5,7a-hexahydro-1H-cyclopenta[b]pyridine;

(cis)-7-(6-bromo-5-methyl-3-pyridinyl)-2,3,4,4a,5,7a-hexahydro-1H-cyclopenta[b]pyridine;

(cis)-7-(5-methoxy-6-methyl-3-pyridinyl)-2,3,4,4a,5,7a-hexahydro-1H-cyclopenta[b]pyridine;

(cis)-7-(6-chloro-5-methoxy-3-pyridinyl)-2,3,4,4a,5,7a-hexahydro-1H-cyclopenta[b]pyridine;

(cis)-7-(6-bromo-5-methoxy-3-pyridinyl)-2,3,4,4a,5,7a-hexahydro-1H-cyclopenta[b]pyridine;

(cis)-7-(3-methyl-5-isoxazolyl)-2,3,4,4a,5,7a-hexahydro-1H-cyclopenta[b]pyridine;

(cis)-2-(2,3,4,4a,5,7a-hexahydro-1H-cyclopenta[b]pyridin-7-yl)furo[3,2-b]pyridine;

(cis)-7-(3-pyridinyl)-1,2,3,4,4a,5,6,8a-octahydroquinoline;

(cis)-7-(6-chloro-3-pyridinyl)-1,2,3,4,4a,5,6,8a-octahydroquinoline;

(cis)-7-(6-bromo-3-pyridinyl)-1,2,3,4,4a,5,6,8a-octahydroquinoline;

(cis)-7-(6-fluoro-3-pyridinyl)-1,2,3,4,4a,5,6,8a-octahydroquinoline;

(cis)-7-(6-methyl-3-pyridinyl)-1,2,3,4,4a,5,6,8a-octahydroquinoline;

(cis)-5-(1,2,3,4,4a,5,6,8a-octahydro-7-quinolinyl)nicotinonitrile;

(cis)-7-(5-methyl-3-pyridinyl)-1,2,3,4,4a, 5,6,8a-octahydroquinoline;

(cis)-7-(5-chloro-3-pyridinyl)-1,2,3,4,4a, 5,6,8a-octahydroquinoline;

(cis)-7-(5-fluoro-3-pyridinyl)-1,2,3,4,4a, 5,6,8a-octahydroquinoline;

(cis)-7-(5-bromo-3-pyridinyl)-1,2,3,4,4a, 5,6,8a-octahydroquinoline;

(cis)-7-(5-vinyl-3-pyridinyl)-1,2,3,4,4a, 5,6,8a-octahydroquinoline;

(cis)-7-(5-methoxy-3-pyridinyl)-1,2,3,4,4a, 5,6,8a-octahydroquinoline;

(cis)-7-(5-vinyloxy-3-pyridinyl)-1,2,3,4,4a,5,6,8a-octahydroquinoline;

(cis)-7-(5-ethynyl-3-pyridinyl)-1,2,3,4,4a, 5,6,8a-octahydroquinoline;

(cis)-2-methyl-5-(1,2,3,4,4a,5,6,8a-octahydro-7-quinolinyl)nicotinonitrile;

(cis)-2-bromo-5-(1,2,3,4,4a,5,6,8a-octahydro-7-quinolinyl)nicotinonitrile;

(cis)-2-chloro-5-(1,2,3,4,4a,5,6,8a-octahydro-7-quinolinyl)nicotinonitrile;

(cis)-7-(5,6-dichloro-3-pyridinyl)-1,2,3,4,4a,5,6,8a-octahydroquinoline;

(cis)-7-(6-bromo-5-chloro-3-pyridinyl)-1,2,3,4,4a,5,6,8a-octahydroquinoline;

(cis)-7-(6-chloro-5-methyl-3-pyridinyl)-1,2,3,4,4a,5,6,8a-octahydroquinoline;

(cis)-7-(6-bromo-5-methyl-3-pyridinyl)-1,2,3,4,4a,5,6,8a-octahydroquinoline;

(cis)-7-(5-methoxy-6-methyl-3-pyridinyl)-1,2,3,4,4a,5,6,8a-octahydroquinoline;

(cis)-7-(6-chloro-5-methoxy-3-pyridinyl)-1,2,3,4,4a,5,6,8a-octahydroquinoline;

(cis)-7-(6-bromo-5-methoxy-3-pyridinyl)-1,2,3,4,4a,5,6,8a-octahydroquinoline;

(cis)-7-furo[3,2-b]pyridin-2-yl-1,2,3,4,4a,5,6,8a-octahydroquinoline;

(cis)-7-(3-methyl-5-isoxazolyl)-1,2,3,4,4a,5,6,8a-octahydroquinoline;

(cis)-6-(3-pyridinyl)-2,3,4,4a,5,7a-hexahydro-1H-cyclopenta[b]pyridine;

(cis)-6-(5-chloro-3-pyridinyl)-2,3,4,4a,5,7a-hexahydro-1H-cyclopenta[b]pyridine;

(cis)-6-(5-bromo-3-pyridinyl)-2,3,4,4a,5,7a-hexahydro-1H-cyclopenta[b]pyridine;

(cis)-6-(5-fluoro-3-pyridinyl)-2,3,4,4a, 5,7a-hexahydro-1H-cyclopenta[b]pyridine;

(cis)-6-(5-methyl-3-pyridinyl)-2,3,4,4a,5,7a-hexahydro-1H-cyclopenta[b]pyridine;

(cis)-5-(2,3,4,4a,5,7a-hexahydro-1H-cyclopenta[b]pyridin-6-yl)nicotinonitrile;

(cis)-6-(5-methyl-3-pyridinyl)-2,3,4,4a,5,7a-hexahydro-1H-cyclopenta[b]pyridine;

(cis)-6-(5-chloro-3-pyridinyl)-2,3,4,4a,5,7a-hexahydro-1H-cyclopenta[b]pyridine;

(cis)-6-(5-fluoro-3-pyridinyl)-2,3,4,4a,5,7a-hexahydro-1H-cyclopenta[b]pyridine;

(cis)-6-(5-bromo-3-pyridinyl)-2,3,4,4a,5,7a-hexahydro-1H-cyclopenta[b]pyridine;

(cis)-6-(5-vinyl-3-pyridinyl)-2,3,4,4a,5,7a-hexahydro-1H-cyclopenta[b]pyridine;

(cis)-6-(5-methoxy-3-pyridinyl)-2,3,4,4a,5,7a-hexahydro-1H-cyclopenta[b]pyridine;

(cis)-6-(5-vinyloxy-3-pyridinyl)-2,3,4,4a, 5,7a-hexahydro-1H-cyclopenta[b]pyridine;

(cis)-6-(5-ethynyl-3-pyridinyl)-2,3,4,4a,5,7a-hexahydro-1H-cyclopenta[b]pyridine;

(cis)-6-(5,6-dichloro-3-pyridinyl)-2,3,4,4a,5,7a-hexahydro-1H-cyclopenta[b]pyridine;

(cis)-6-(6-bromo-5-chloro-3-pyridinyl)-2,3,4,4a,5,7a-hexahydro-1H-cyclopenta[b]pyridine;

(cis)-5-(2,3,4,4a,5,7a-hexahydro-1H-cyclopenta[b]pyridin-6-yl)-2-methylnicotinonitrile;

(cis)-2-bromo-5-(2,3,4,4a,5,7a-hexahydro-1H-cyclopenta[b]pyridin-6-yl)nicotinonitrile;

(cis)-2-chloro-5-(2,3,4,4a,5,7a-hexahydro-1H-cyclopenta[b]pyridin-6-yl)nicotinonitrile;

(cis)-6-(6-chloro-5-methyl-3-pyridinyl)-2,3,4,4a,5,7a-hexahydro-1H-cyclopenta[b]pyridine;

(cis)-6-(6-bromo-5-methyl-3-pyridinyl)-2,3,4,4a,5,7a-hexahydro-1H-cyclopenta[b]pyridine;

(cis)-6-(5-methoxy-6-methyl-3-pyridinyl)-2,3,4,4a,5,7a-hexahydro-1H-cyclopenta[b]pyridine;

(cis)-6-(6-chloro-5-methoxy-3-pyridinyl)-2,3,4,4a,5,7a-hexahydro-1H-cyclopenta[b]pyridine;

(cis)-6-(6-bromo-5-methoxy-3-pyridinyl)-2,3,4,4a,5,7a-hexahydro-1H-cyclopenta[b]pyridine;

(cis)-6-(3-methyl-5-isoxazolyl)-2,3,4,4a,5,7a-hexahydro-1H-cyclopenta[b]pyridine;

(cis)-2-(2,3,4,4a,5,7a-hexahydro-1H-cyclopenta[b]pyridin-6-yl)furo[3,2-b]pyridine;

(cis)-7-(3-pyridinyl)-1,2,3,4,4a,5,8,8a-octahydroquinoline;

(cis)-7-(6-bromo-3-pyridinyl)-1,2,3,4,4a,5,8,8a-octahydroquinoline;

(cis)-7-(6-chloro-3-pyridinyl)-1,2,3,4,4a,5,8,8a-octahydroquinoline;

(cis)-7-(6-fluoro-3-pyridinyl)-1,2,3,4,4a,5,8,8a-octahydroquinoline;

(cis)-7-(6-methyl-3-pyridinyl)-1,2,3,4,4a,5,8,8a-octahydroquinoline;

(cis)-5-(1,2,3,4,4a,5,8,8a-octahydro-7-quinolinyl)nicotinonitrile;

(cis)-7-(5-methyl-3-pyridinyl)-1,2,3,4,4a,5,8,8a-octahydroquinoline;

(cis)-7-(5-chloro-3-pyridinyl)-1,2,3,4,4a,5,8,8a-octahydroquinoline;

(cis)-7-(5-fluoro-3-pyridinyl)-1,2,3,4,4a,5,8,8a-octahydroquinoline;

(cis)-7-(5-bromo-3-pyridinyl)-1,2,3,4,4a, 5,8,8a-octahydroquinoline;

(cis)-7-(5-vinyl-3-pyridinyl)-1,2,3,4,4a,5,8,8a-octahydroquinoline;

(cis)-7-(5-methoxy-3-pyridinyl)-1,2,3,4,4a,5,8,8a-octahydroquinoline;

(cis)-7-(5-vinyloxy-3-pyridinyl)-1,2,3,4,4a,5,8,8a-octahydroquinoline;

(cis)-7-(5-ethynyl-3-pyridinyl)-1,2,3,4,4a,5,8,8a-octahydroquinoline;

(cis)-7-(5,6-dichloro-3-pyridinyl)-1,2,3,4,4a,5,8,8a-octahydroquinoline;

(cis)-7-(6-bromo-5-chloro-3-pyridinyl)-1,2,3,4,4a,5,8,8a-octahydroquinoline;

(cis)-2-methyl-5-(1,2,3,4,4a,5,8,8a-octahydro-7-quinolinyl)nicotinonitrile;

(cis)-2-bromo-5-(1,2,3,4,4a,5,8,8a-octahydro-7-quinolinyl)nicotinonitrile;

(cis)-2-chloro-5-(1,2,3,4,4a,5,8,8a-octahydro-7-quinolinyl)nicotinonitrile;

(cis)-7-(6-chloro-5-methyl-3-pyridinyl)-1,2,3,4,4a,5,8,8a-octahydroquinoline;

(cis)-7-(6-bromo-5-methyl-3-pyridinyl)-1,2,3,4,4a,5,8,8a-octahydroquinoline;

(cis)-7-(5-methoxy-6-methyl-3-pyridinyl)-1,2,3,4,4a,5,8,8a-octahydroquinoline;

(cis)-7-(6-chloro-5-methoxy-3-pyridinyl)-1,2,3,4,4a,5,8,8a-octahydroquinoline;

(cis)-7-(6-bromo-5-methoxy-3-pyridinyl)-1,2,3,4,4a,5,8,8a-octahydroquinoline;

(cis)-7-(3-methyl-5-isoxazolyl)-1,2,3,4,4a,5,8,8a-octahydroquinoline;

(cis)-7-furo[3,2-b]pyridin-2-yl-1,2,3,4,4a,5,8,8a-octahydroquinoline;

(cis)-6-(3-pyridinyl)-2,3,4,4a,7,7a-hexahydro-1H-cyclopenta[b]pyridine;

(cis)-6-(6-chloro-3-pyridinyl)-2,3,4,4a,7,7a-hexahydro-1H-cyclopenta[b]pyridine;

(cis)-6-(6-fluoro-3-pyridinyl)-2,3,4,4a,7,7a-hexahydro-1H-cyclopenta[b]pyridine;

(cis)-6-(6-bromo-3-pyridinyl)-2,3,4,4a,7,7a-hexahydro-1H-cyclopenta[b]pyridine;

(cis)-6-(6-methyl-3-pyridinyl)-2,3,4,4a,7,7a-hexahydro-1H-cyclopenta[b]pyridine;

(cis)-5-(2,3,4,4a,7,7a-hexahydro-1H-cyclopenta[b]pyridin-6-yl)nicotinonitrile;

(cis)-6-(5-chloro-3-pyridinyl)-2,3,4,4a,7,7a-hexahydro-1H-cyclopenta[b]pyridine;

(cis)-6-(5-methyl-3-pyridinyl)-2,3,4,4a,7,7a-hexahydro-1H-cyclopenta[b]pyridine;

(cis)-6-(5-bromo-3-pyridinyl)-2,3,4,4a,7,7a-hexahydro-1H-cyclopenta[b]pyridine;

(cis)-6-(5-fluoro-3-pyridinyl)-2,3,4,4a,7,7a-hexahydro-1H-cyclopenta[b]pyridine;

(cis)-6-(5-vinyl-3-pyridinyl)-2,3,4,4a,7,7a-hexahydro-1H-cyclopenta[b]pyridine;

(cis)-6-(5-methoxy-3-pyridinyl)-2,3,4,4a,7,7a-hexahydro-1H-cyclopenta[b]pyridine;

(cis)-6-(5-vinyloxy-3-pyridinyl)-2,3,4,4a,7,7a-hexahydro-1H-cyclopenta[b]pyridine;

(cis)-6-(5-ethynyl-3-pyridinyl)-2,3,4,4a,7,7a-hexahydro-1H-cyclopenta[b]pyridine;

(cis)-6-(5,6-dichloro-3-pyridinyl)-2,3,4,4a,7,7a-hexahydro-1H-cyclopenta[b]pyridine;

(cis)-6-(6-bromo-5-chloro-3-pyridinyl)-2,3,4,4a,7,7a-hexahydro-1H-cyclopenta[b]pyridine;

(cis)-5-(2,3,4,4a,7,7a-hexahydro-1H-cyclopenta[b]pyridin-6-yl)-2-methylnicotinonitrile;

(cis)-2-bromo-5-(2,3,4,4a,7,7a-hexahydro-1H-cyclopenta[b]pyridin-6-yl)nicotinonitrile;

(cis)-2-chloro-5-(2,3,4,4a,7,7a-hexahydro-1H-cyclopenta[b]pyridin-6-yl)nicotinonitrile;

(cis)-6-(6-chloro-5-methyl-3-pyridinyl)-2,3,4,4a,7,7a-hexahydro-1H-cyclopenta[b]pyridine;

(cis)-6-(6-bromo-5-methyl-3-pyridinyl)-2,3,4,4a,7,7a-hexahydro-1H-cyclopenta[b]pyridine;

(cis)-6-(5-methoxy-6-methyl-3-pyridinyl)-2,3,4,4a,7,7a-hexahydro-1H-cyclopenta[b]pyridine;

(cis)-6-(6-chloro-5-methoxy-3-pyridinyl)-2,3,4,4a,7,7a-hexahydro-1H-cyclopenta[b]pyridine;

(cis)-6-(6-bromo-5-methoxy-3-pyridinyl)-2,3,4,4a,7,7a-hexahydro-1H-cyclopenta[b]pyridine;

(cis)-6-(3-methyl-5-isoxazolyl)-2,3,4,4a,7,7a-hexahydro-1H-cyclopenta[b]pyridine;

(cis)-2-(2,3,4,4a,7,7a-hexahydro-1H-cyclopenta[b]pyridin-6-yl)furo[3,2-b]pyridine; or a pharmaceutically acceptable salt, amide, ester and prodrug thereof.

In another embodiment of the present invention, compounds of formula (VI) are disclosed

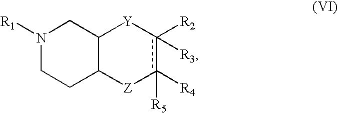

or a pharmaceutically acceptable salt, amide, ester and prodrug thereof wherein Y, Z, R₁, R₂, R₃, R₄ and R₅ are as defined in formula (I).

In another embodiment of the present invention, compounds of formula (VI) are disclosed wherein Y is a covalent bond; Z is a covalent bond; and R₁, R₂, R₃, R₄, and R₅ are as defined in formula (I).

In another embodiment of the present invention, compounds of formula (VI) are disclosed wherein Y is a covalent bond; Z is CH₂; and R₁, R₂, R₃, R₄, and R₅ are as defined in formula (I).

In another embodiment of the present invention, compounds of formula (VI) are disclosed wherein [symbol] represents a double bond; Y is a covalent bond; Z is CH₂; R₂ is hydrogen; R₃ and R₅ are absent; R₄ is heterocycle; and R₁ is as defined in formula (I).

In another embodiment of the present invention, compounds of formula (VI) are disclosed wherein [symbol] represents a double bond; Y is a covalent bond; Z is CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is a covalent bond; Z is CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (VI) are disclosed wherein [symbol] represents a double bond; Y is a covalent bond; Z is CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (VI) are disclosed wherein [symbol] represents a double bond; Y is a covalent bond; Z is CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (VI) are disclosed wherein [symbol] represents a double bond; Y is a covalent bond; Z is CH₂; R₂ is heterocycle; R₃ and R₅ are absent; R₄ is hydrogen; and R₁ is as defined in formula (I).

In another embodiment of the present invention, compounds of formula (VI) are disclosed wherein [symbol] represents a double bond; Y is a covalent bond; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is a covalent bond; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (VI) are disclosed wherein [symbol] represents a double bond; Y is a covalent bond; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (VI) are disclosed wherein [symbol] represents a double bond; Y is a covalent bond; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (VI) are disclosed wherein Y is CH₂; Z is a covalent bond; and R₁, R₂, R₃, R₄, and R₅ are as defined in formula (I).

In another embodiment of the present invention, compounds of formula (VI) are disclosed wherein [symbol] represents a double bond; Y is CH₂; Z is a covalent bond; R₂ is heterocycle; R₃ and R₅ are absent; R₄ is hydrogen; and R₁ is as defined in formula (I).

In another embodiment of the present invention, compounds of formula (VI) are disclosed wherein [symbol] represents a double bond; Y is CH₂; Z is a covalent bond; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is CH₂; Z is a covalent bond; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (VI) are disclosed wherein [symbol] represents a double bond; Y is CH₂; Z is a covalent bond; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (VI) are disclosed wherein [symbol] represents a double bond; Y is CH₂; Z is a covalent bond; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (VI) are disclosed wherein Y is a covalent bond; Z is CH₂CH₂; and R₁, R₂, R₃, R₄, and R₅ are as defined in formula (I).

In another embodiment of the present invention, compounds of formula (VI) are disclosed wherein [symbol] represents a double bond; Y is a covalent bond; Z is CH₂CH₂; R₂ is hydrogen; R₃ and R₅ are absent; R₄ is heterocycle; and R₁ is as defined in formula (I).

In another embodiment of the present invention, compounds of formula (VI) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; and R₁, R₁₀, and R₁₁ are as defined in formula (I).

In another embodiment of the present invention, compounds of formula (VI) are disclosed wherein [symbol] represents a double bond; Y is a covalent bond; Z is CH₂CH₂; R₂ is hydrogen; R₃ and R₅ are absent; R₄ is heterocycle wherein the heterocycle is pyridinyl substituted with 0, 1, or 2 substituents independently selected from alkenyl, alkenyloxy, alkoxy, alkyl, alkynyl, cyano, halogen, or nitro; and R₁ is as defined in formula (I).

In another embodiment of the present invention, compounds of formula (VI) are disclosed wherein [symbol] represents a double bond; Y is a covalent bond; Z is CH₂CH₂; R₂ is hydrogen; R₃ and R₅ are absent; R₄ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; and R₁ is as defined in formula (I).

In another embodiment of the present invention, compounds of formula (VI) are disclosed wherein [symbol] represents a double bond; Y is a covalent bond; Z is CH₂CH₂; R₂ is heterocycle; R₃ and R₅ are absent; R₄ is hydrogen; and R₁ is as defined in formula (I).

In another embodiment of the present invention, compounds of formula (VI) are disclosed wherein [symbol] represents a double bond; Y is a covalent bond; Z is CH₂CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is a covalent bond; Z is CH₂CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (VI) are disclosed wherein [symbol] represents a double bond; Y is a covalent bond; Z is CH₂CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (VI) are disclosed wherein [symbol] represents a double bond; Y is a covalent bond; Z is CH₂CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (VI) are disclosed wherein Y is CH₂; Z is CH₂; and R₁, R₂, R₃, R₄, and R₅ are as defined in formula (I).

In another embodiment of the present invention, compounds of formula (VI) are disclosed wherein [symbol] represents a double bond; Y is CH₂; Z is CH₂; R₂ is heterocycle; R₃ and R₅ are absent; R₄ is hydrogen; and R₁ is as defined in formula (I).

In another embodiment of the present invention, compounds of formula (VI) are disclosed wherein [symbol] represents a double bond; Y is CH₂; Z is CH₂; R₂ is heterocycle wherein the heterocycle is selected from furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl or thieno[2,3-b]pyridinyl wherein the heterocycle is substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, or —S(O)₂R₁₂; R₃ and R₅ are absent; R₄ is hydrogen; R₁₂, R₁₃, and R₁₄ are independently selected from hydrogen, alkyl, aryl, or arylalkyl; and R₁, R₁₀, and R₁₁ are as defined in formula (I).

In another embodiment of the present invention, compounds of formula (VI) are disclosed wherein [symbol] represents a double bond; Y is CH₂; Z is CH₂; R₂ is heterocycle wherein the heterocycle is selected from imidazolyl, isoxazolyl, pyridinyl, tetrazolyl, thiadiazolyl, thiazolyl, thienyl, imidazo[1,2-a]pyridinyl, thieno[3,2-b]pyridinyl or thieno[2,3-b]pyridinyl wherein the heterocycle is substituted with 0, 1, or 2 substituents independently selected from alkenyl, alkenyloxy, alkoxy, alkyl, alkynyl, cyano, halogen, nitro, (NR₁₀R₁₁)sulfonyl, or —C(NH)NR₁₀R₁₁; R₃ and R₅ are absent; R₄ is hydrogen; and R₁, R₁₀, and R₁₁ are as defined in formula (I).

In another embodiment of the present invention, compounds of formula (VI) are disclosed wherein [symbol] represents a double bond; Y is CH₂; Z is CH₂; R₂ is heterocycle wherein the heterocycle is pyridinyl substituted with 0, 1, or 2 substituents independently selected from alkenyl, alkenyloxy, alkoxy, alkyl, alkynyl, cyano, halogen, or nitro; R₃ and R₅ are absent; R₄ is hydrogen; and R₁ is as defined in formula (I).

In another embodiment of the present invention, compounds of formula (VI) are disclosed wherein [symbol] represents a double bond; Y is CH₂; Z is CH₂; R₂ is heterocycle wherein the heterocycle is selected from 3-pyridinyl, 6-bromo-3-pyridinyl, 6-chloro-3-pyridinyl, 6-fluoro-3 pyridinyl, 6-methyl-3-pyridinyl, 5-cyano-3-pyridinyl, 5-methyl-3-pyridinyl, 5-chloro-3-pyridinyl, 5-fluoro-3-pyridinyl, 5-bromo-3-pyridinyl, 5-vinyl-3-pyridinyl, 5-methoxy-3-pyridinyl, 5-vinyloxy-3-pyridinyl, 5-ethynyl-3-pyridinyl, 5,6-dichloro-3-pyridinyl, 6-bromo-5-chloro-3-pyridinyl, 5-cyano-6-methyl-3-pyridinyl, 6-chloro-5-cyano-3-pyridinyl, 6-bromo-5-cyano-3-pyridinyl, 6-chloro-5-methyl-3-pyridinyl, 6-bromo-5-methyl-3-pyridinyl, 5-methoxy-6-methyl-3-pyridinyl, 6-chloro-5-methoxy-3-pyridinyl, or 6-bromo-5-methoxy-3-pyridinyl; R₃ and R₅ are absent; R₄ is hydrogen; and R₁ is as defined in formula (I).

In another embodiment of the present invention, compounds of formula (VI) are disclosed wherein Y is CH₂CH₂; Z is a covalent bond; and R₁, R₂, R₃, R₄, and R₅ are as defined in formula (I).

Representative compounds of formula (VI) include, but are not limited to:

(cis)-8-(3-pyridinyl)-1,2,3,4,4a,5,6,8a-octahydroisoquinoline;

(cis)-8-(6-chloro-3-pyridinyl)-1,2,3,4,4a,5,6,8a-octahydroisoquinoline;

(cis)-8-(6-bromo-3-pyridinyl)-1,2,3,4,4a,5,6,8a-octahydroisoquinoline;

(cis)-8-(6-fluoro-3-pyridinyl)-1,2,3,4,4a,5,6,8a-octahydroisoquinoline;

(cis)-8-(6-methyl-3-pyridinyl)-1,2,3,4,4a,5,6,8a-octahydroisoquinoline;

(cis)-5-(1,2,3,4,4a,5,6,8a-octahydro-8-isoquinolinyl)nicotinonitrile;

(cis)-8-(5-chloro-3-pyridinyl)-1,2,3,4,4a,5,6,8a-octahydroisoquinoline;

(cis)-8-(5-bromo-3-pyridinyl)-1,2,3,4,4a,5,6,8a-octahydroisoquinoline;

(cis)-8-(5-fluoro-3-pyridinyl)-1,2,3,4,4a,5,6,8a-octahydroisoquinoline;

(cis)-8-(5-methyl-3-pyridinyl)-1,2,3,4,4a,5,6,8a-octahydroisoquinoline;

(cis)-8-(5-vinyl-3-pyridinyl)-1,2,3,4,4a,5,6,8a-octahydroisoquinoline;

(cis)-8-(5-methoxy-3-pyridinyl)-1,2,3,4,4a,5,6,8a-octahydroisoquinoline;

(cis)-8-(5-vinyloxy-3-pyridinyl)-1,2,3,4,4a,5,6,8a-octahydroisoquinoline;

(cis)-8-(5-ethynyl-3-pyridinyl)-1,2,3,4,4a,5,6,8a-octahydroisoquinoline;

(cis)-8-(5,6-dichloro-3-pyridinyl)-1,2,3,4,4a,5,6,8a-octahydroisoquinoline;

(cis)-8-(6-bromo-5-chloro-3-pyridinyl)-1,2,3,4,4a, 5,6, 8a-octahydroisoquinoline;

(cis)-2-methyl-5-(1,2,3,4,4a,5,6,8a-octahydro-8-isoquinolinyl)nicotinonitrile;

(cis)-2-bromo-5-(1,2,3,4,4a,5,6,8a-octahydro-8-isoquinolinyl)nicotinonitrile;

(cis)-2-chloro-5-(1,2,3,4,4a,5,6,8a-octahydro-8-isoquinolinyl)nicotinonitrile;

(cis)-8-(6-chloro-5-methyl-3-pyridinyl)-1,2,3,4,4a,5,6,8a-octahydroisoquinoline;

(cis)-8-(6-bromo-5-methyl-3-pyridinyl)-1,2,3,4,4a,5,6,8a-octahydroisoquinoline;

(cis)-8-(5-methoxy-6-methyl-3-pyridinyl)-1,2,3,4,4a,5,6,8a-octahydroisoquinoline;

(cis)-8-(6-bromo-5-methoxy-3-pyridinyl)-1,2,3,4,4a,5,6,8a-octahydroisoquinoline;

(cis)-8-(6-chloro-5-methoxy-3-pyridinyl)-1,2,3,4,4a,5,6,8a-octahydroisoquinoline;

(cis)-8-(3-methyl-5-isoxazolyl)-1,2,3,4,4a,5,6,8a-octahydroisoquinoline;

(cis)-8-furo[3,2-b]pyridin-2-yl-1,2,3,4,4a, 5,6,8a-octahydroisoquinoline;

(cis)-7-(3-pyridinyl)-2,3,4,4a,5,7a-hexahydro-1H-cyclopenta[c]pyridine;

(cis)-7-(6-chloro-3-pyridinyl)-2,3,4,4a,5,7a-hexahydro-1H-cyclopenta[c]pyridine;

(cis)-7-(6-bromo-3-pyridinyl)-2,3,4,4a,5,7a-hexahydro-1H-cyclopenta[c]pyridine;

(cis)-7-(6-fluoro-3-pyridinyl)-2,3,4,4a,5,7a-hexahydro-1H-cyclopenta[c]pyridine;

(cis)-7-(6-methyl-3-pyridinyl)-2,3,4,4a,5,7a-hexahydro-1H-cyclopenta[c]pyridine;

(cis)-5-(2,3,4,4a,5,7a-hexahydro-1H-cyclopenta[c]pyridin-7-yl)nicotinonitrile;

(cis)-7-(5-methyl-3-pyridinyl)-2,3,4,4a,5,7a-hexahydro-1H-cyclopenta[c]pyridine;

(cis)-7-(5-chloro-3-pyridinyl)-2,3,4,4a, 5,7a-hexahydro-1H-cyclopenta[c]pyridine;

(cis)-7-(5-bromo-3-pyridinyl)-2,3,4,4a, 5,7a-hexahydro-1H-cyclopenta[c]pyridine;

(cis)-7-(5-fluoro-3-pyridinyl)-2,3,4,4a,5,7a-hexahydro-1H-cyclopenta[c]pyridine;

(cis)-7-(5-vinyl-3-pyridinyl)-2,3,4,4a,5,7a-hexahydro-1H-cyclopenta[c]pyridine;

(cis)-7-(5-methoxy-3-pyridinyl)-2,3,4,4a,5,7a-hexahydro-1H-cyclopenta[c]pyridine;

(cis)-7-(5-vinyloxy-3-pyridinyl)-2,3,4,4a,5,7a-hexahydro-1H-cyclopenta[c]pyridine;

(cis)-7-(5-ethynyl-3-pyridinyl)-2,3,4,4a,5,7a-hexahydro-1H-cyclopenta[c]pyridine;

(cis)-5-(2,3,4,4a,5,7a-hexahydro-1H-cyclopenta[c]pyridin-7-yl)-2-methylnicotinonitrile;

(cis)-2-bromo-5-(2,3,4,4a,5,7a-hexahydro-1H-cyclopenta[c]pyridin-7-yl)nicotinonitrile;

(cis)-2-chloro-5-(2,3,4,4a,5,7a-hexahydro-1H-cyclopenta[c]pyridin-7-yl)nicotinonitrile;

(cis)-7-(5,6-dichloro-3-pyridinyl)-2,3,4,4a,5,7a-hexahydro-1H-cyclopenta[c]pyridine;

(cis)-7-(6-bromo-5-chloro-3-pyridinyl)-2,3,4,4a,5,7a-hexahydro-1H-cyclopenta[c]pyridine;

(cis)-7-(6-chloro-5-methyl-3-pyridinyl)-2,3,4,4a, 5,7a-hexahydro-1H-cyclopenta[c]pyridine;

(cis)-7-(6-bromo-5-methyl-3-pyridinyl)-2,3,4,4a,5,7a-hexahydro-1H-cyclopenta[c]pyridine;

(cis)-7-(5-methoxy-6-methyl-3-pyridinyl)-2,3,4,4a, 5,7a-hexahydro-1H-cyclopenta[c]pyridine;

(cis)-7-(6-chloro-5-methoxy-3-pyridinyl)-2,3,4,4a,5,7a-hexahydro-1H-cyclopenta[c]pyridine;

(cis)-7-(6-bromo-5-methoxy-3-pyridinyl)-2,3,4,4a, 5,7a-hexahydro-1H-cyclopenta[c]pyridine;

(cis)-2-(2,3,4,4a,5,7a-hexahydro-1H-cyclopenta[c]pyridin-7-yl)furo[3,2-b]pyridine;

(cis)-7-(3-methyl-5-isoxazolyl)-2,3,4,4a,5,7a-hexahydro-1H-cyclopenta[c]pyridine;

(cis)-7-(3-pyridinyl)-1,2,3,4,4a, 5,6,8a-octahydroisoquinoline;

(cis)-7-(6-chloro-3-pyridinyl)-1,2,3,4,4a,5,6,8a-octahydroisoquinoline;

(cis)-7-(6-bromo-3-pyridinyl)-1,2,3,4,4a,5,6,8a-octahydroisoquinoline;

(cis)-7-(6-fluoro-3-pyridinyl)-1,2,3,4,4a,5,6,8a-octahydroisoquinoline;

(cis)-7-(6-methyl-3-pyridinyl)-1,2,3,4,4a,5,6,8a-octahydroisoquinoline;

(cis)-5-(1,2,3,4,4a,5,6,8a-octahydro-7-isoquinolinyl)nicotinonitrile;

(cis)-7-(5-methyl-3-pyridinyl)-1,2,3,4,4a, 5,6,8a-octahydroisoquinoline;

(cis)-7-(5-chloro-3-pyridinyl)-1,2,3,4,4a, 5,6,8a-octahydroisoquinoline;

(cis)-7-(5-bromo-3-pyridinyl)-1,2,3,4,4a,5,6,8a-octahydroisoquinoline;

(cis)-7-(5-fluoro-3-pyridinyl)-1,2,3,4,4a,5,6,8a-octahydroisoquinoline;

(cis)-7-(5-vinyl-3-pyridinyl)-1,2,3,4,4a,5,6,8a-octahydroisoquinoline;

(cis)-7-(5-methoxy-3-pyridinyl)-1,2,3,4,4a, 5,6,8a-octahydroisoquinoline;

(cis)-7-(5-vinyloxy-3-pyridinyl)-1,2,3,4,4a,5,6,8a-octahydroisoquinoline;

(cis)-7-(5-ethynyl-3-pyridinyl)-1,2,3,4,4a, 5,6,8a-octahydroisoquinoline;

(cis)-7-(5,6-dichloro-3-pyridinyl)-1,2,3,4,4a,5,6,8a-octahydroisoquinoline;

(cis)-7-(6-bromo-5-chloro-3-pyridinyl)-1,2,3,4,4a, 5,6,8a-octahydroisoquinoline;

(cis)-2-methyl-5-(1,2,3,4,4a,5,6,8a-octahydro-7-isoquinolinyl)nicotinonitrile;

(cis)-2-bromo-5-(1,2,3,4,4a,5,6,8a-octahydro-7-isoquinolinyl)nicotinonitrile;

(cis)-2-chloro-5-(1,2,3,4,4a,5,6,8a-octahydro-7-isoquinolinyl)nicotinonitrile;

(cis)-7-(6-chloro-5-methyl-3-pyridinyl)-1,2,3,4,4a, 5,6,8a-octahydroisoquinoline;

(cis)-7-(6-bromo-5-methyl-3-pyridinyl)-1,2,3,4,4a,5,6,8a-octahydroisoquinoline;

(cis)-7-(5-methoxy-6-methyl-3-pyridinyl)-1,2,3,4,4a,5,6,8a-octahydroisoquinoline;

(cis)-7-(6-chloro-5-methoxy-3-pyridinyl)-1,2,3,4,4a,5,6,8a-octahydroisoquinoline;

(cis)-7-(6-bromo-5-methoxy-3-pyridinyl)-1,2,3,4,4a,5,6,8a-octahydroisoquinoline;

(cis)-8-(3-methyl-5-isoxazolyl)-1,2,3,4,4a,5,6,8a-octahydroisoquinoline;

(cis)-8-furo[3,2-b]pyridin-2-yl-1,2,3,4,4a,5,6,8a-octahydroisoquinoline;

(cis)-6-(3-pyridinyl)-2,3,4,4a,5,7a-hexahydro-1H-cyclopenta[c]pyridine;

(cis)-6-(6-chloro-3-pyridinyl)-2,3,4,4a,5,7a-hexahydro-1H-cyclopenta[c]pyridine;

(cis)-6-(6-bromo-3-pyridinyl)-2,3,4,4a,5,7a-hexahydro-1H-cyclopenta[c]pyridine;

(cis)-6-(6-fluoro-3-pyridinyl)-2,3,4,4a,5,7a-hexahydro-1H-cyclopenta[c]pyridine;

(cis)-6-(6-methyl-3-pyridinyl)-2,3,4,4a,5,7a-hexahydro-1H-cyclopenta[c]pyridine;

(cis)-5-(2,3,4,4a,5,7a-hexahydro-1H-cyclopenta[c]pyridin-6-yl)nicotinonitrile;

(cis)-6-(5-chloro-3-pyridinyl)-2,3,4,4a,5,7a-hexahydro-1H-cyclopenta[c]pyridine;

(cis)-6-(5-bromo-3-pyridinyl)-2,3,4,4a,5,7a-hexahydro-1H-cyclopenta[c]pyridine;

(cis)-6-(5-fluoro-3-pyridinyl)-2,3,4,4a,5,7a-hexahydro-1H-cyclopenta[c]pyridine;

(cis)-6-(5-methyl-3-pyridinyl)-2,3,4,4a,5,7a-hexahydro-1H-cyclopenta[c]pyridine;

(cis)-6-(5-vinyl-3-pyridinyl)-2,3,4,4a, 5,7a-hexahydro-1H-cyclopenta[c]pyridine;

(cis)-6-(5-methoxy-3-pyridinyl)-2,3,4,4a,5,7a-hexahydro-1H-cyclopenta[c]pyridine;

(cis)-6-(5-vinyloxy-3-pyridinyl)-2,3,4,4a,5,7a-hexahydro-1H-cyclopenta[c]pyridine;

(cis)-6-(5-ethynyl-3-pyridinyl)-2,3,4,4a,5,7a-hexahydro-1H-cyclopenta[c]pyridine;

(cis)-6-(5,6-dichloro-3-pyridinyl)-2,3,4,4a,5,7a-hexahydro-1H-cyclopenta[c]pyridine;

(cis)-6-(6-bromo-5-chloro-3-pyridinyl)-2,3,4,4a,5,7a-hexahydro-1H-cyclopenta[c]pyridine;

(cis)-5-(2,3,4,4a,5,7a-hexahydro-1H-cyclopenta[c]pyridin-6-yl)-2-methylnicotinonitrile;

(cis)-2-chloro-5-(2,3,4,4a,5,7a-hexahydro-1H-cyclopenta[c]pyridin-6-yl)nicotinonitrile;

(cis)-2-bromo-5-(2,3,4,4a,5,7a-hexahydro-1H-cyclopenta[c]pyridin-6-yl)nicotinonitrile;

(cis)-6-(6-chloro-5-methyl-3-pyridinyl)-2,3,4,4a,5,7a-hexahydro-1H-cyclopenta[c]pyridine;

(cis)-6-(6-bromo-5-methyl-3-pyridinyl)-2,3,4,4a,5,7a-hexahydro-1H-cyclopenta[c]pyridine;

(cis)-6-(5-methoxy-6-methyl-3-pyridinyl)-2,3,4,4a,5,7a-hexahydro-1H-cyclopenta[c]pyridine;

(cis)-6-(6-bromo-5-methoxy-3-pyridinyl)-2,3,4,4a,5,7a-hexahydro-1H-cyclopenta[c]pyridine;

(cis)-6-(6-chloro-5-methoxy-3-pyridinyl)-2,3,4,4a,5,7a-hexahydro-1H-cyclopenta[c]pyridine;

(cis)-6-(3-methyl-5-isoxazolyl)-2,3,4,4a,5,7a-hexahydro-1H-cyclopenta[c]pyridine;

(cis)-2-(2,3,4,4a,5,7a-hexahydro-1H-cyclopenta[c]pyridin-6-yl)furo[3,2-b]pyridine;

(cis)-7-(3-pyridinyl)-1,2,3,4,4a,5,8,8a-octahydroisoquinoline;

(cis)-7-(6-methyl-3-pyridinyl)-1,2,3,4,4a,5,8,8a-octahydroisoquinoline;

(cis)-7-(6-chloro-3-pyridinyl)-1,2,3,4,4a,5,8,8a-octahydroisoquinoline;

(cis)-7-(6-bromo-3-pyridinyl)-1,2,3,4,4a, 5,8,8a-octahydroisoquinoline;

(cis)-7-(6-fluoro-3-pyridinyl)-1,2,3,4,4a, 5,8,8a-octahydroisoquinoline;

(cis)-5-(1,2,3,4,4a,5,8,8a-octahydro-7-isoquinolinyl)nicotinonitrile;

(cis)-7-(5-methyl-3-pyridinyl)-1,2,3,4,4a,5,8,8a-octahydroisoquinoline;

(cis)-7-(5-chloro-3-pyridinyl)-1,2,3,4,4a,5,8,8a-octahydroisoquinoline;

(cis)-7-(5-bromo-3-pyridinyl)-1,2,3,4,4a,5,8,8a-octahydroisoquinoline;

(cis)-7-(5-fluoro-3-pyridinyl)-1,2,3,4,4a,5,8,8a-octahydroisoquinoline;

(cis)-7-(5,6-dichloro-3-pyridinyl)-1,2,3,4,4a,5,8,8a-octahydroisoquinoline;

(cis)-7-(6-bromo-5-chloro-3-pyridinyl)-1,2,3,4,4a, 5,8, 8a-octahydroisoquinoline;

(cis)-2-methyl-5-(1,2,3,4,4a,5,8,8a-octahydro-7-isoquinolinyl)nicotinonitrile;

(cis)-2-chloro-5-(1,2,3,4,4a,5,8,8a-octahydro-7-isoquinolinyl)nicotinonitrile;

(cis)-2-bromo-5-(1,2,3,4,4a,5,8,8a-octahydro-7-isoquinolinyl)nicotinonitrile;

(cis)-7-(6-chloro-5-methyl-3-pyridinyl)-1,2,3,4,4a,5,8,8a-octahydroisoquinoline;

(cis)-7-(6-bromo-5-methyl-3-pyridinyl)-1,2,3,4,4a,5,8,8a-octahydroisoquinoline;

(cis)-7-(5-methoxy-6-methyl-3-pyridinyl)-1,2,3,4,4a,5,8,8a-octahydroisoquinoline;

(cis)-7-(6-chloro-5-methoxy-3-pyridinyl)-1,2,3,4,4a,5,8,8a-octahydroisoquinoline;

(cis)-7-(6-bromo-5-methoxy-3-pyridinyl)-1,2,3,4,4a,5,8,8a-octahydroisoquinoline;

(cis)-7-(3-methyl-5-isoxazolyl)-1,2,3,4,4a,5,8,8a-octahydroisoquinoline;

(cis)-7-furo[3,2-b]pyridin-2-yl-1,2,3,4,4a,5,8,8a-octahydroisoquinoline;

(cis)-6-(3-pyridinyl)-2,3,4,4a,7,7a-hexahydro-1H-cyclopenta[c]pyridine;

(cis)-6-(6-chloro-3-pyridinyl)-2,3,4,4a,7,7a-hexahydro-1H-cyclopenta[c]pyridine;

(cis)-6-(6-bromo-3-pyridinyl)-2,3,4,4a,7,7a-hexahydro-1H-cyclopenta[c]pyridine;

(cis)-6-(6-fluoro-3-pyridinyl)-2,3,4,4a,7,7a-hexahydro-1H-cyclopenta[c]pyridine;

(cis)-6-(6-methyl-3-pyridinyl)-2,3,4,4a,7,7a-hexahydro-1H-cyclopenta[c]pyridine;

(cis)-5-(2,3,4,4a,7,7a-hexahydro-1H-cyclopenta[c]pyridin-6-yl)nicotinonitrile;

(cis)-6-(5-chloro-3-pyridinyl)-2,3,4,4a,7,7a-hexahydro-1H-cyclopenta[c]pyridine;

(cis)-6-(5-bromo-3-pyridinyl)-2,3,4,4a,7,7a-hexahydro-1H-cyclopenta[c]pyridine;

(cis)-6-(5-fluoro-3-pyridinyl)-2,3,4,4a,7,7a-hexahydro-1H-cyclopenta[c]pyridine;

(cis)-6-(5-methyl-3-pyridinyl)-2,3,4,4a,7,7a-hexahydro-1H-cyclopenta[c]pyridine;

(cis)-6-(5—vinyl-3-pyridinyl)-2,3,4,4a,7,7a-hexahydro-1H-cyclopenta[c]pyridine;

(cis)-6-(5-methoxy-3-pyridinyl)-2,3,4,4a,7,7a-hexahydro-1H-cyclopenta[c]pyridine;

(cis)-6-(5-vinyloxy-3-pyridinyl)-2,3,4,4a,7,7a-hexahydro-1H-cyclopenta[c]pyridine;

(cis)-6-(5-ethynyl-3-pyridinyl)-2,3,4,4a,7,7a-hexahydro-1H-cyclopenta[c]pyridine;

(cis)-6-(5,6-dichloro-3-pyridinyl)-2,3,4,4a,7,7a-hexahydro-1H-cyclopenta[c]pyridine;

(cis)-6-(6-bromo-5-chloro-3-pyridinyl)-2,3,4,4a,7,7a-hexahydro-1H-cyclopenta[c]pyridine;

(cis)-5-(2,3,4,4a,7,7a-hexahydro-1H-cyclopenta[c]pyridin-6-yl)-2-methylnicotinonitrile;

(cis)-2-chloro-5-(2,3,4,4a,7,7a-hexahydro-1H-cyclopenta[c]pyridin-6-yl)nicotinonitrile;

(cis)-2-bromo-5-(2,3,4,4a,7,7a-hexahydro-1H-cyclopenta[c]pyridin-6-yl)nicotinonitrile;

(cis)-6-(6-bromo-5-methyl-3-pyridinyl)-2,3,4,4a,7,7a-hexahydro-1H-cyclopenta[c]pyridine;

(cis)-6-(6-chloro-5-methyl-3-pyridinyl)-2,3,4,4a,7,7a-hexahydro-1H-cyclopenta[c]pyridine;

(cis)-6-(5-methoxy-6-methyl-3-pyridinyl)-2,3,4,4a,7,7a-hexahydro-1H-cyclopenta[c]pyridine;

(cis)-6-(6-chloro-5-methoxy-3-pyridinyl)-2,3,4,4a,7,7a-hexahydro-1H-cyclopenta[c]pyridine;

(cis)-6-(6-bromo-5-methoxy-3-pyridinyl)-2,3,4,4a,7,7a-hexahydro-1H-cyclopenta[c]pyridine;

(cis)-6-(3-methyl-5-isoxazolyl)-2,3,4,4a,7,7a-hexahydro-1H-cyclopenta[c]pyridine;

(cis)-2-(2,3,4,4a,7,7a-hexahydro-1H-cyclopenta[c]pyridin-6-yl)furo[3,2-b]pyridine; or a pharmaceutically acceptable salt, amide, ester and prodrug thereof.

Another embodiment of the present invention relates to pharmaceutical compositions comprising a therapeutically effective amount of a compound of formula (I-VI) or a pharmaceutically acceptable salt thereof. The compostion may be in combination with a pharmaceutically acceptable carrier.

Another embodiment of the present invention relates to a method of treating a disorder, such as Alzheimer's disease, Parkinson's disease, memory dysfunction, Tourette's syndrome, sleep disorders, attention deficit hyperactivity disorder, neurodegeneration, inflammation, neuroprotection, amyotrophic lateral sclerosis, anxiety, depression, mania, schizophrenia, nicotinic withdrawal syndrome, anorexia and other eating disorders, AIDS-induced dementia, epilepsy, urinary incontinence, substance abuse, smoking cessation and inflammatory bowel syndrome, in a mammal in need of such treatment comprising administering to the mammal a therapeutically effective amount of a compound of formula (I-VI) or a pharmaceutically acceptable salt, amide, ester or prodrug thereof.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof in combination with a pharmaceutically acceptable carrier.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof in combination with an opioid.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof in combination with a non-steroid anti-inflammatory agent.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof in combination with a tricyclic antidepressant.

Another embodiment of the present invention relates to a method for treating pain in a mammal in need of such treatment comprising administering to the mammal a therapeutically effective amount of a compound of formula (I-VI) or a pharmaceutically acceptable salt, amide, ester or prodrug thereof in combination with an anticonvulsant such as gabapentin or pregabalin.

Definition of Terms

As used throughout this specification and the appended claims, the following terms have the following meanings.

The term “alkenyl” as used herein, means a straight or branched chain hydrocarbon containing from 2 to 10 carbons, preferably 2 to 6 carbon atoms, preferably in a straight chain, and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, 2-(sec-butylcarbonyl)ethyl, 2-(isopropoxycarbonyl)ethyl, and 2-(tert-butoxycarbonyl)ethyl.

The term “alkyl” as used herein, means a straight or branched chain hydrocarbon containing from 1 to 10 carbon atoms, preferably 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxy moiety, as defined herein. Representative examples of alkylcarbonyloxy include, but are not limited to, acetyloxy, ethylcarbonyloxy, and tert-butylcarbonyloxy.

The term “alkylthio” as used herein, means an alkyl group, as defined herein, appended to the parent molecular moiety through a sulfur atom: Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms, preferably 2 to 6 carbon atoms, preferably in a straight chain, and containing at least one carbon-carbon triple bond. Representative examples of alkynyl include, but are not limited, to acetylenyl, 1-propynyl, 2-propynyl, 3-butynyl, 2-pentynyl, 3-methyl-1-pentynyl, 3,4-dimethyl-1-pentynyl, and 1-butynyl.

The term “aryl” as used herein, means a monocyclic-ring system, or a fused bicyclic-ring system wherein one or more of the fused rings are aromatic. Representative examples of aryl include, but are not limited to, azulenyl, indanyl, indenyl, naphthyl, phenyl, and tetrahydronaphthyl.

The aryl groups of this invention are substituted with 0, 1, 2, 3, 4 or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂ and —S(O)₂R₁₂ wherein R₁₂, R₁₃ and R₁₄ are independently selected from hydrogen, alkyl, alkylcarbonyl, aryl, and arylalkyl, as defined herein. The aryl groups of this invention can be further substituted with an additional aryl group, as defined herein, or an additional heterocycle, as defined herein, wherein the additional aryl group and the additional heterocycle are substituted with 0, 1, 2 or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂ and —S(O)₂R₁₂ wherein R₁₂, R₁₃ and R₁₄ are independently selected from hydrogen, alkyl, alkylcarbonyl, aryl, and arylalkyl, as defined herein.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, 1,2-difluoroethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic” as used herein, means a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepinyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above heterocyclic ring systems fused to an aryl group as defined herein, a cycloalkyl group as defined herein, or another heterocyclic ring system. Representative examples of bicyclic ring systems include but are not limited to, for example, benzimidazolyl, benzothiazolyl, benzothienyl, benzoxazolyl, benzofuranyl, benzopyranyl, benzothiopyranyl, benzodioxinyl, cinnolinyl, furopyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, pyranopyridyl, quinolinyl, quinolizinyl, quinoxalinyl, quinazolinyl, tetrahydroisoquinolinyl, tetrahydroquinolinyl, thienopyridinyl, thieno[3,2-b]pyridinyl, thieno[2,3-b]pyridinyl and thiopyranopyridyl. Tricyclic rings systems are exemplified by any of the above bicyclic ring systems fused to an aryl group as defined herein, a cycloalkyl group as defined herein, or a heterocyclic ring system. Representative examples of tricyclic ring systems include, but are not limited to, acridinyl, carbazolyl, carbolinyl, dibenzo[b,d]furanyl, dibenzo[b,d]thienyl, naphtho[2,3-b]furan, naphtho[2,3-b]thienyl, phenazinyl, phenothiazinyl, phenoxazinyl, thianthrenyl, thioxanthenyl and xanthenyl.

The heterocycles of this invention are substituted with 0, 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂ wherein R₁₂, R₁₃, and R₁₄ are independently selected from hydrogen, alkyl, alkylcarbonyl, aryl, and arylalkyl as defined herein. The heterocycles of this invention can be further substituted with an additional aryl group, as defined herein, or an additional heterocycle, as defined herein, wherein the additional aryl group and the additional heterocycle can be substituted with 1, 2 or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀ R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂ wherein R₁₂, R₁₃, and R₁₄ are independently selected from hydrogen, alkyl, aryl, and arylalkyl, as defined herein.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypropyl, and 2-ethyl-4-hydroxyheptyl.

The term “mercapto” as used herein, means a —SH group.

The term “mercaptoalkyl” as used herein, means at least one mercapto group, as defined herein, appended to the parent molecular moiety through an alkyl group, as defined herein. Representative examples of mercaptoalkyl include, but are not limited to, thiomethyl, 2-thioethyl and 3-thiopropyl.

The term “—NR₁₀R₁₁” as used herein, means two groups, R₁₀ and R₁₁, which are appended to the parent molecular moiety through a nitrogen atom. R₁₀ and R₁₁ are independently selected from hydrogen, alkyl, alkylcarbonyl, aryl, and arylalkyl as defined herein. Representative examples of —NR₁₀R₁₁ include, but are not limited to, acetylamino, amino, benzylamino, methylamino, dimethylamino, ethylamino, phenylamino, and methylcarbonylamino.

The term “(NR₁₀R₁₁)alkyl” as used herein, means a —NR₁₀R₁₁, as defined herein, appended to the parent molecular moiety through an alkyl group, as defined herein. Representative examples of (NR₁₀R₁₁)alkyl include, but are not limited, aminomethyl, (methylamino)methyl, 2-aminoethyl, and (dimethylamino)methyl.

The term “(NR₁₀R₁₁)carbonyl” as used herein, means a —NR₁₀R₁₁ group, as defined herein, appended to the parent molecular moiety through a carbonyl group, as defined herein. Representative examples of (NR₁₀R₁₁)carbonyl include, but are not limited to, aminocarbonyl, dimethylaminocarbonyl, methylaminocarbonyl, and ethylaminocarbonyl.

The term “(NR₁₀R₁₁)carbonylalkyl” as used herein, means a (NR₁₀R₁₁)carbonyl group, as defined herein, appended to the parent molecular moiety through an alkyl group, as defined herein. Representative examples of (NR₁₀R₁₁)carbonylalkyl include, but are not limited to, 2-amino-2-oxoethyl, 2-(methylamino)-2-oxoethyl, 4-amino-4-oxobutyl, and 4-(dimethylamino)-4-oxobutyl.

The term “(NR₁₀R₁₁)sulfonyl” as used herein, means a —NR₁₀R₁₁ group, as defined herein, appended to the parent molecular moiety through a sulfonyl group, as defined herein. Representative examples of (NR₁₀R₁₁)sulfonyl include, but are not limited to, aminosulfonyl, dimethylaminosulfonyl, methylaminosulfonyl, ethylaminosulfonyl, phenylaminosulfonyl and benzylaminosulfonyl.

The term “nitrogen protecting group” or “N-protecting group” as used herein, means those groups intended to protect an amino group against undesirable reactions during synthetic procedures. Nitrogen protecting groups comprise carbamates, amides, N-benzyl derivatives, and imine derivatives. Preferred nitrogen protecting groups are acetyl, benzoyl, benzyl, benzyloxycarbonyl (Cbz), formyl, phenylsulfonyl, pivaloyl, tert-butoxycarbonyl (Boc), trifluoroacetyl, and triphenylmethyl (trityl).

The term “nitro” as used herein, means a —NO₂ group.

The term “oxo” as used herein, means a ═O moiety.

The term “oxy” as used herein, means a —O— moiety.

The term “sulfonyl” as used herein, means a —SO₂— group.

Compounds of the present invention were named by ACD/ChemSketch version 5.0 (developed by Advanced Chemistry Development, Inc., Toronto, ON, Canada) or were given names which appeared to be consistent with ACD nomenclature.

It is to be understood that compounds of the present invention can be either cis or trans and that the cis and trans arrangements are included within the scope of the present invention.

The present invention contemplates stereoisomers and mixtures thereof which are specifically included within the scope of this invention. Stereoisomers include enantiomers, diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

### Determination of Biological Activity In Vitro Data Determination of Nicotinic Acetylcholine Receptor Binding Potencies

Compounds of the present invention were subjected to an in vitro assay against the nicotinic acetylcholine receptor as described below and were found to be effective binders to the receptor. The In Vitro protocols for determination of nicotinic acetylcholine channel receptor binding potencies of ligands were determined as follows.

Binding of [³H]-cytisine ([³H]-CYT) to neuronal nicotinic acetylcholine receptors was accomplished using crude synaptic membrane preparations from whole rat brain (Pabreza et al., Molecular Pharmacol., 1990, 39:9). Washed membranes were stored at −80° C. prior to use. Frozen aliquots were slowly thawed and resuspended in 20 volumes of buffer (containing: 120 mM NaCl, 5 mM KCl, 2 mM MgCl₂, 2 mM CaCl₂ and 50 mM Tris-Cl, pH 7.4 @4° C.). After centrifuging at 20,000×g for 15 minutes, the pellets were resuspended in 30 volumes of buffer.

The test compounds were dissolved in water to make 10 mM stock solutions. This solution was then diluted (1:100) with buffer (as above) and further taken through seven serial log dilutions to produce test solutions from 10⁻⁵ to 10⁻¹¹ M.

Homogenate (containing 125-150 μg protein) was added to triplicate tubes containing the range of concentrations of test compound described above and [³H]-CYT (1.25 mM) in a final volume of 500 μL. Samples were incubated for 60 minutes at 4° C., then rapidly filtered through Whatman GF/B filters presoaked in 0.5% polyethyleneimine using 3×4 mL of ice-cold buffer. The filters are counted in 4 mL of Ecolume® (ICN). Nonspecific binding was determined in the presence of 10 μM (−)-nicotine and values were expressed as a percentage of total binding. IC₅₀ values were determined with the RS-1 (BBN) nonlinear least squares curve-fitting program and IC₅₀ values were converted to Ki values using the Cheng and Prusoff correction (Kᵢ=IC₅₀/(1+[ligand]/Kd of ligand).

Representative compounds of the present invention bound to nicotinic acetylcholine receptors with binding affinities from 2300 nM to 0.029 nM. Preferred compounds of the present invention bound to nicotinic acetylcholine receptors with binding affinities less than or equal to 100 nM.

### In Vivo Data Determination of Effectiveness of Nicotinic Acetylcholine Receptor Ligands as Analgesic Agents in the Mouse Hot Plate Paradigm

An in vivo protocol was utilized to determine the effectiveness of nicotinic acetylcholine receptor ligands as analgesic agents in the mouse hot plate paradigm.

Separate groups of mice, (n=8/group) were utilized for dose group. All drugs were administered by the intraperitoneal route of administration. Test drugs were dissolved in water to make a 6.2 mM stock solution. Animals were dosed with this solution (10 mL/kg body weight) for a 62 micromol/kg dose. Lower doses were administered similarly, following serial dilution of the stock solution in half-log increments. Animals were dosed 30 minutes prior to testing in the hot plate. The hot-plate utilized was an automated analgesia monitor (Model #AHP 16AN, Omnitech Electronics, Inc. of Columbus, Ohio). The temperature of the hot plate was maintained at 55° C. and a cut-off time of 180 seconds was utilized. Latency until the tenth jump was recorded as the dependent measure. An increase in the tenth jump latency relative to the control was considered an effect.

Representative compounds of the present invention showed an antinociceptive effect in the mouse hot plate paradigm at doses ranging from 62 μmol/kg to 6.2 μmol/kg. Preferred compounds of the present invention showed an antinociceptive effect in the mouse hot plate paradigm at doses less than or equal to 62 μmol/kg.

### Determination of Effectiveness of Nicotinic Acetylcholine Receptor Ligands as Analgesic Agents in the Rat Formalin Test

Another in vivo protocol utilized to determine the effectiveness of nicotinic acetylcholine receptor ligands as analgesic agents was the rat formalin test.

Male Sprague-Dawley rats (Charles River, Portage, Mich.) weighing 200 to 400 grams were used for all experiments. After a 20 minute period of acclimation to individual cages, 50 μL of a 5% formalin solution was injected subcutaneous into the dorsal aspect of one of the rear paws and the rats were then returned to the clear observation cages suspended above mirror panels. Rats were observed during phase 2 of the formalin test which was defined as the 20 minute period from 30 to 50 minutes after formalin injection. The investigator recorded nocifensive behaviors in the injected paw of four animals during the session by observing the animals for one 15 second observation period during each 1 minute interval. Nocifensive behaviors recorded included flinching, licking or biting the injected paw. In dose-response studies, the test compound (or saline) was administered intraperitoneally 5 minutes before injection of formalin.

Representative compounds of the present invention showed an antinociceptive effect in the rat formalin test at doses ranging from 62 μmol/kg to 1.9 μmol/kg. Preferred compounds of the present invention showed an antinociceptive effect in the rat formalin test at doses less than or equal to 62 μmol/kg.

The in vitro and in vivo data demonstrates that compounds of the present invention bind to the nicotinic acetylcholine receptor and are useful for treating pain.

The ability of the compounds of the present invention, including but not limited to those specified in the examples, to treat pain via the nicotinic acetylcholine receptors and the cholinergic system can be further demonstrated by Williams, M.; Arneric, S. P.: Beyond the Tobacco Debate: dissecting out the therapeutic potential of nicotine. Exp. Opin. Invest. Drugs (1996)5(8): 1035-1045; and Arneric, S. P.; Sullivan, J. P.; Williams, W.: Neuronal nicotinic acetylcholine receptors. Novel targets for central nervous system theraputics in: Psychopharmacology: The Fourth Generation of Progress. Bloom F E, Kupfer D J (Eds.), Raven Press, New York (1995): 95-109.

Additionally, compounds of the present invention are useful for ameliorating or preventing disorders affected by nicotinic acetylcholine receptors and the cholinergic system, such as Alzheimer's disease, Parkinson's disease, memory dysfunction, Tourette's syndrome, sleep disorders, attention deficit hyperactivity disorder, neurodegeneration, inflammation, neuroprotection, anxiety, depression, mania, schizophrenia, anorexia and other eating disorders, AIDS-induced dementia, epilepsy, urinary incontinence, substance abuse, smoking cessation and inflammatory bowel syndrome.

The ability of the compounds of the present invention, including but not limited to those specified in the examples, to treat Alzheimer's disease can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100; Lindstrom, J.: Nicotinic Acetylchloline Receptors in Health and Disease. Molecular Neurobiology (1997) 15: 193-222; and Lloyd, G K; Menzaghi, F; Bontempi B; Suto, C; Siegel, R; Akong, M; Stauderman, K; Velicelebi, G; Johnson, E; Harpold, M M; Rao, T S; Sacaan, A I; Chavez-Noriega, L E; Washburn, M S; Vernier, J M; Cosford, N D P; McDonald, L A: The potential of subtype selective neuronal nicotinic acetylcholine receptor agonists as therapeutic agents. Life Sciences (1998)62(17/18): 1601-1606.

The ability of the compounds of the present invention, including but not limited to those specified in the examples, to treat Parkinson's disease can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Lindstrom, J.: Nicotinic Acetylchloline Receptors in Health and Disease. Molecular Neurobiology (1997) 15: 193-222; and Lloyd, G K; Menzaghi, F; Bontempi B; Suto, C; Siegel, R; Akong, M; Stauderman, K; Velicelebi, G; Johnson, E; Harpold, M M; Rao, T S; Sacaan, A I; Chavez-Noriega, L E; Washburn, M S; Vernier, J M; Cosford, N D P; McDonald, L A: The potential of subtype selective neuronal nicotinic acetylcholine receptor agonists as therapeutic agents. Life Sciences (1998)62(17/18): 1601-1606.

The ability of the compounds of the present invention, including but not limited to those specified in the examples, to treat memory dysfunction can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat Tourette's syndrome can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat sleeping disorders can be demonstrated by Williams, M.; Arneric, S. P.: Beyond the Tobacco Debate: dissecting out the therapeutic potential of nicotine. Exp. Opin. Invest. Drugs (1996)5(8): 1035-1045.

The ability of the compounds of the present invention, including but not limited to those specified in the examples, to treat attention deficit hyperactivity disorder can be demonstrated by Williams, M.; Arneric, S. P.: Beyond the Tobacco Debate: dissecting out the therapeutic potential of nicotine. Exp. Opin. Invest. Drugs (1996)5(8): 1035-1045; and Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100.

The ability of the compounds of the present invention, including but not limited to those specified in the examples, to treat neurodegeneration and to provide neuroprotection can be demonstrated by Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100.

The ability of the compounds of the present invention, including but not limited to those specified in the examples, to treat inflammation can be demonstrated by Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100.

The ability of the compounds of the present invention, including but not limited to those specified in the examples, to treat amyotrophic lateral sclerosis can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100.

The ability of the compounds of the present invention, including but not limited to those specified in the examples, to treat anxiety can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Arneric, S. P.; Holladay, M. W.; Sullivan, J. P.: Cholinergic channel modulators as a novel therapeutic strategy for Alzheimer's disease. Exp. Opin. Invest. Drugs (1996) 5(1): 79-100.

The ability of the compounds of the present invention, including but not limited to those specified in the examples, to treat depression can be demonstrated by Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109.

The ability of the compounds of the present invention, including but not limited to those specified in the examples, to treat mania and schizophrenia can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat anorexia and other eating disorders can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat AIDS-induced dementia can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat epilepsy can be demonstrated by Williams, M.; Arneric, S. P.: Beyond the Tobacco Debate: dissecting out the therapeutic potential of nicotine. Exp. Opin. Invest. Drugs (1996)5(8): 1035-1045; Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109; and Lindstrom, J.: Nicotinic Acetylchloline Receptors in Health and Disease. Molecular Neurobiology (1997) 15: 193-222.

The ability of the compounds of the present invention, including but not limited to those specified in the examples, to treat urinary incontinence can be demonstrated by Williams, M.; Arneric, S. P.: Beyond the Tobacco Debate: dissecting out the therapeutic potential of nicotine. Exp. Opin. Invest. Drugs (1996)5(8): 1035-1045.

The ability of the compounds of the present invention, including but not limited to those specified in the examples, to treat premenstrual syndrome can be demonstrated by Williams, M.; Arneric, S. P.: Beyond the Tobacco Debate: dissecting out the therapeutic potential of nicotine. Exp. Opin. Invest. Drugs (1996)5(8): 1035-1045; and Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109.

The ability of the compounds of the present invention, including but not limited to those specified in the examples, to treat substance abuse can be demonstrated by Williams, M.; Arneric, S. P.: Beyond the Tobacco Debate: dissecting out the therapeutic potential of nicotine. Exp. Opin. Invest. Drugs (1996)5(8): 1035-1045; and Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109.

The ability of the compounds of the present invention, including but not limited to those specified in the examples, to treat smoking cessation can be demonstrated by Williams, M.; Arneric, S. P.: Beyond the Tobacco Debate: dissecting out the therapeutic potential of nicotine. Exp. Opin. Invest. Drugs (1996)5(8): 1035-1045; and Arneric, S. P.; Sullivan, J. P.; Williams, W.: Neuronal nicotinic acetylcholine receptors. Novel targets for central nervous system theraputics. in: Psychopharmacology: The Fourth Generation of Progress. Bloom F E, Kupfer D J (Eds.), Raven Press, New York (1995): 95-109.

The ability of the compounds of the present invention, including but not limited to those specified in the examples, to treat inflammatory bowel syndrome can be demonstrated by Williams, M.; Arneric, S. P.: Beyond the Tobacco Debate: dissecting out the therapeutic potential of nicotine. Exp. Opin. Invest. Drugs (1996)5(8): 1035-1045; and Lindstrom, J.: Nicotinic Acetylchloline Receptors in Health and Disease. Molecular Neurobiology (1997) 15: 193-222.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

Dosage forms for topical administration of a compound of the present invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions, and mode of administration.

When used in the above or other treatments, a therapeutically effective amount of one of the compounds of the present invention can be employed in pure form or, where such forms exist, in pharmaceutically acceptable salt, ester, amide, or prodrug form. Alternatively, the compound can be administered as a pharmaceutical composition containing the compound of interest in combination with one or more pharmaceutically acceptable carriers. The phrase “therapeutically effective amount” of the compound of the present invention means a sufficient amount of the compound to treat disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of the present invention administered to a mammal, and particularly a human, may range from about 0.01 to about 50 mg/kg/day. More preferable doses can be in the range of from about 0.01 to about 5 mg/kg/day. If desired, the effective daily dose can be divided into multiple doses for purposes of administration; consequently, single dose compositions may contain such amounts or submultiples thereof to make up the daily dose.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The present invention contemplates pharmaceutically active compounds either chemically synthesized or formed by in vivo biotransformation to compounds of formula (I-VI).

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The term “pharmaceutically acceptable salt, ester, amide, and prodrug,” as used herein, refers to carboxylate salts, amino acid addition salts, zwitterions, esters, amides, and prodrugs of compounds of formula (I-VI) which are within the scope of sound medical judgement, suitable for use in contact with the tissues of humans and lower animals without undue toxicity, irritation, allergic response, and the like, are commensurate with a reasonable benefit/risk ratio, and are effective for their intended use. Representative examples include, but are not limited to, 5-(1,2,3,3a,4,6a-hexahydrocyclopenta[c]pyrrol-5-yl)-3-pyridinyl acetate, 5-(1,2,3,3a,4,6a-hexahydrocyclopenta[c]pyrrol-5-yl)-3-pyridinyl benzoate, 2-chloro-5-(1,2,3,3a,4,6a-hexahydrocyclopenta[c]pyrrol-5-yl)-3-pyridinyl benzoate, 2-chloro-5-(1,2,3,3a,4,6a-hexahydrocyclopenta[c]pyrrol-5-yl)-3-pyridinyl acetate, [2-chloro-5-(1,2,3,3a,4,6a-hexahydrocyclopenta[c]pyrrol-5-yl)-3-pyridinyl]methyl benzoate, [2-chloro-5-(1,2,3,3a,4,6a-hexahydrocyclopenta[c]pyrrol-5-yl)-3-pyridinyl]methyl acetate, [5-(1,2,3,3a,4,6a-hexahydrocyclopenta[c]pyrrol-5-yl)-3-pyridinyl]methyl acetate, [5-(1,2,3,3a,4,6a-hexahydrocyclopenta[c]pyrrol-5-yl)-3-pyridinyl]methyl benzoate, 2-acetyl-5-(3-pyridinyl)-1,2,3,3a,4,6a-hexahydrocyclopenta[c]pyrrole, 2-acetyl-5-(6-chloro-3-pyridinyl)-1,2,3,3a,4,6a-hexahydrocyclopenta[c]pyrrole, 5-(6-chloro-3-pyridinyl)-2-(trifluoroacetyl)-1,2,3,3a,4,6a-hexahydrocyclopenta[c]pyrrole, 5-(3-pyridinyl)-2-(trifluoroacetyl)-1,2,3,3a,4,6a-hexahydrocyclopenta[c]pyrrole, 2-benzoyl-5-(3-pyridinyl)-1,2,3,3a,4,6a-hexahydrocyclopenta[c]pyrrole, 2-benzoyl-5-(6-chloro-3-pyridinyl)-1,2,3,3a,4,6a-hexahydrocyclopenta[c]pyrrole, phenyl 5-(6-chloro-3-pyridinyl)-3,3a,4,6a-tetrahydrocyclopenta[c]pyrrole-2(1H)-carboxylate, and phenyl 5-(3-pyridinyl)-3,3a,4,6a-tetrahydrocyclopenta[c]pyrrole-2(1H)-carboxylate.

The compounds of the present invention can be used in the form of pharmaceutically acceptable salts derived from inorganic or organic acids. The term “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as maleic acid, fumaric acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the present invention include phosphate, tris and acetate.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I-VI), for example, by hydrolysis in blood.

The term “pharmaceutically acceptable ester” or “ester,” as used herein, refers to esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula (I-VI) may be prepared according to conventional methods.

The term “pharmaceutically acceptable amide” or “amide,” as used herein, refers to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I-VI) may be prepared according to conventional methods.

### Abbreviations

Abbreviations which have been used in the descriptions of the Schemes and the Examples that follow are: Ac for acetyl; acyl for alkylcarbonyl; AIBN for 2,2′-azobis(2-methylpropionitrile) N,N-dimethylformamide; Bn for benzyl; dppf for 1,1′-bis(diphenylphosphino)ferrocene; DMAP for dimethylaminopyridine; DME for 1,2-dimethoxyethane; DMSO for dimethylsulfoxide; EtOAc for ethyl acetate; EtOH for ethanol; formalin for a solution of formaldehyde (37% by weight) in water; HPLC for high pressure liquid chromatography; LAH for lithium aluminum hydride; LDA for lithium diusopropylamine; MeOH for methanol; Ms for mesylate —SO₂CH₃; Tf for (trifluoromethyl)sulfonyl (—SO₂CF₃); TFA for trifluoroacetic acid; THF for tetrahydrofuran; TMS for trimethylsilyl; Ts or tosyl for p-CH₃(C₆H₄)SO₂—; and TsOH for para-toluenesulfonic acid.

### Preparation of Compounds of The Present Invention

The compounds and processes of the present invention will be better understood in connection with the following synthetic schemes and methods which illustrate a means by which the compounds of the invention can be prepared.

The compounds of this invention can be prepared by a variety of procedures and synthetic routes. Representative procedures and synthetic routes are shown in, but are not limited to, Schemes 1-49.

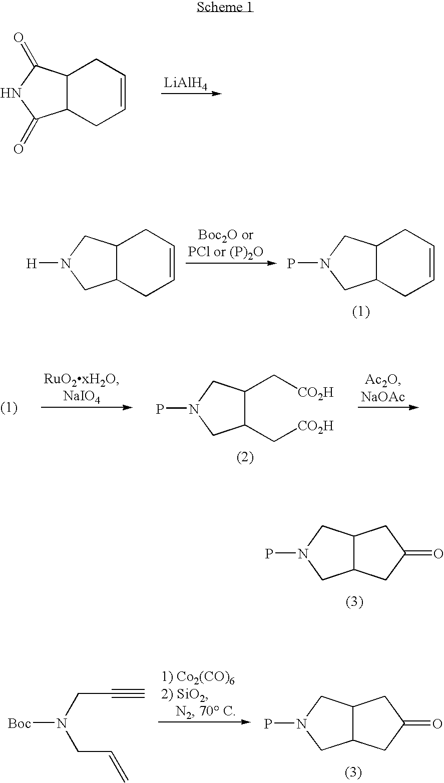

Nitrogen-protected (cis)-hexahydrocyclopenta[c]pyrrol-5(1H)-ones of general formula (3), wherein P is a suitable nitrogen protecting group such as, but not limited to, alkyl, benzyl, triphenylmethyl (trityl), acyl, p-toluenesulfonyl (Ts), benzyloxy carbonyl (Cbz) or tert-butoxycarbonyl (Boc), may be prepared as described in Scheme 1. (cis)-3a,4,7,7a-Tetrahydro-1H-isoindole-1,3(2H)-dione, purchased commercially or prepared as described in Helv. Chim. Acta (1996) 79(3), 875-894 and J. Org. Chem. (1951) 16, 501-505, may be treated with lithium aluminum hydride to provide (cis)-2,3,3a,4,7,7a-hexahydro-1H-isoindole as described in J. Am. Chem. Soc. (1980) 102(6), 2005-2010. (cis)-2,3,3a,4,7,7a-Hexahydro-1H-isoindole may be treated with a nitrogen protecting group precursor such as, but not limited to, di-tert-butyl dicarbonate or benzyl chloroformate to provide nitrogen protected isoindoles of general formula (1). Nitrogen protected isoindoles of general formula (1) may be oxidatively cleaved in the presence of ruthenium (IV) oxide hydrate and sodium periodate as desribed in J. Org. Chem. (1981) 46(19), 3936-3938 and Chem. Pharm. Bull. (1995) 43(8), 1318-1324) to provide diacids of general formula (2). Diacids of general formula (2) may be treated with acetic anhydride and sodium acetate as described in J. Org. Chem. (1989) 54, 5115-5122 to provide nitrogen-protected (cis)-hexahydrocyclopenta[c]pyrrol-5(1H)-ones of general formula (3).

Alternatively, tert-butyl (cis)-5-oxohexahydrocyclopenta[c]pyrrole-2(1H)-carboxylate may be prepared by treating tert-butyl allyl(2-propynyl)carbamate to a reductive Pauson-Khand cyclocarbonylation as described in Tetrahedron (1993) 49(23), 5047-5054. An asymmetric cyclocarbonylation has also been reported in J. Org. Chem. (1999) 64, 5547-5550 that can allow access to analogs in high enantiomeric excess.

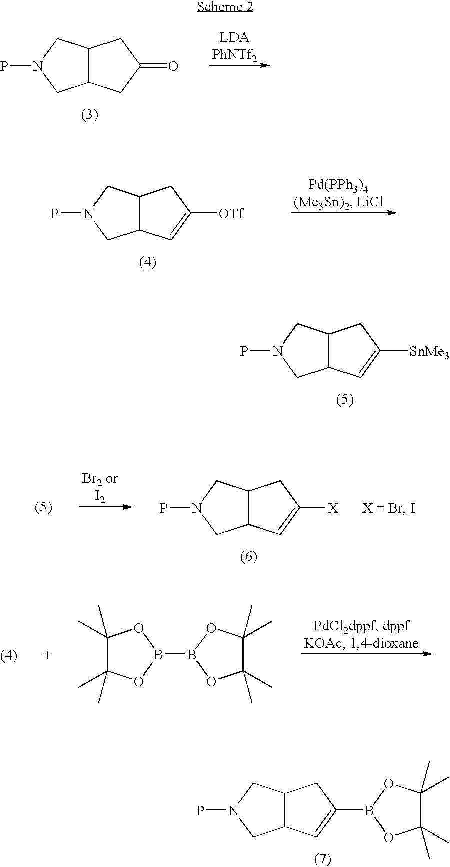

Vinyl triflates of general formula (4), vinyl stannanes of general formula (5), vinyl halides of general formula (6) and vinyl boronates of general formula (7), wherein P is a suitable nitrogen protecting group such as, but not limited to, alkyl, benzyl, trityl, acyl, p-toluenesulfonyl (Ts), benzyloxy carbonyl (Cbz) or tert-butoxycarbonyl (Boc), may be prepared as described in Scheme 2. Nitrogen-protected (cis)-hexahydrocyclopenta[c]pyrrol-5(1H)-ones of general formula (3) may be treated with a base such as, but not limited to, lithium diisopropylamide or sodium bis(trimethylsilyl)amide or potassium bis(trimethylsilyl)amide and N-phenyltrifluormethanesulfonimide to provide vinyl triflates of general formula (4) as described in Tetrahedron Lett. (1983) 24(10), 979-282. Vinyl triflates of general formula (4) may be further elaborated into vinyl stannanes of general formula (5) as described in J. Org. Chem. (1986) 51, 277-279. Vinyl stannanes of general formula (5) may be treated with bromine or iodine to provide vinyl halides of general formula (6) as described in J. Org. Chem. (1985) 50, 2438-2443. Vinyl triflates of general formula (4) may also be converted into vinyl boronates of general formula (7) as described in Tetrahedron Lett. (2000) 41(19), 3705-3708.

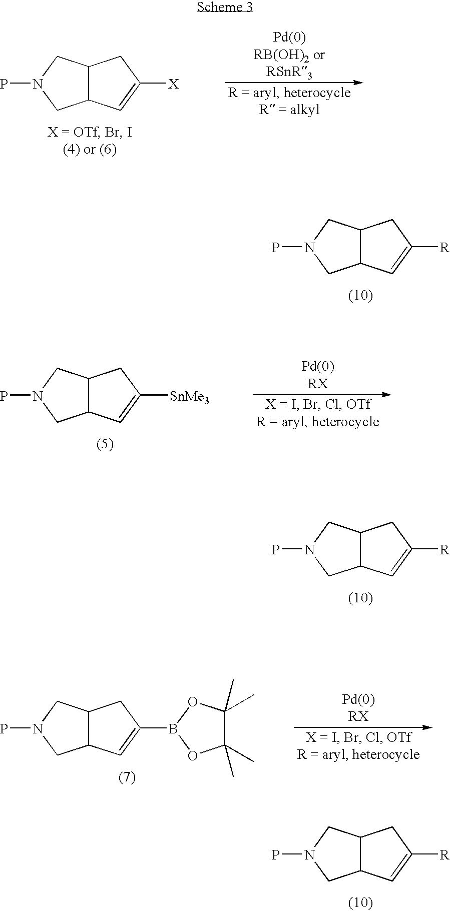

Azabicyclic compounds of general formula (10), wherein P is a suitable nitrogen protecting group such as, but not limited to, alkyl, benzyl, trityl, acyl, p-toluenesulfonyl (Ts) and R is aryl or heterocycle, may be prepared as described in Scheme 3. Vinyl triflates of general formula (4) or vinyl halides of general formula (6) may be treated with a palladium catalyst and an aryl or heterocyclic boronic acid (or an analogous aryl or heterocyclic stannane) to provide azabicyclic compounds of general formula (10). Vinyl stannanes of general formula (5) may be treated with a palladium catalyst and an aryl halide or a heterocyclic halide (or triflate) to provide azabicyclic compounds of general formula (10). Vinyl boronates of general formula (7) may be treated with a palladium catalyst and an aryl halide or a heterocyclic halide (or triflate) to provide azabicyclic compounds of general formula (10).

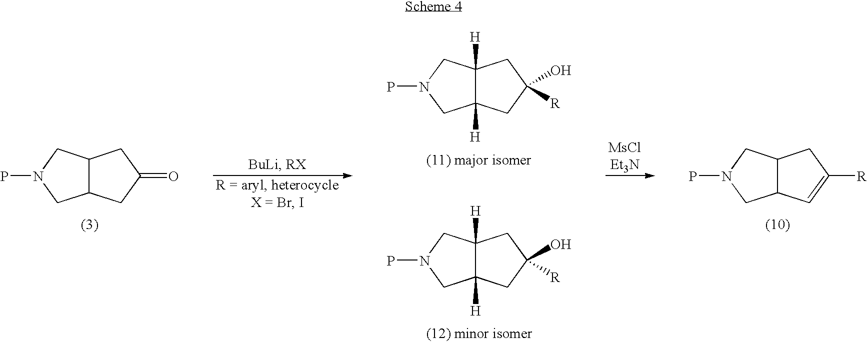

An alternative method of preparing azabicyclic compounds of general formula (10), wherein P is a suitable nitrogen protecting group such as, but not limited to, alkyl, benzyl, trityl, acyl, p-toluenesulfonyl (Ts) and R is aryl or heterocycle, may be used as described in Scheme 4. Nitrogen-protected (cis)-hexahydrocyclopenta[c]pyrrol-5(1H)-ones of general formula (3) may be treated with n-butyllithium or tert-butyllithium and an aryl halide or a heterocyclic halide to provide alcohols of general formula (11) and general formula (12). In general, the major diasteroemer produced in additions of aryl or heterocyclic anions to fused azabicyclic ketones such as, but not limited to, (3) and other related fused bicyclic aminoketones described herein is expected to result from nucleophilic addition to the exo (or convex) face of the azabicyclic ketone as illustrated in Scheme 4. The diastereomers (11) and (12) can be separated with standard chromatographic techniques used by those skilled in the art of organic chemistry. Alcohols of general formula (11) and general formula (12) may be treated with methanesulfonyl chloride and triethylamine to provide azabicyclic compounds of general formula (10).

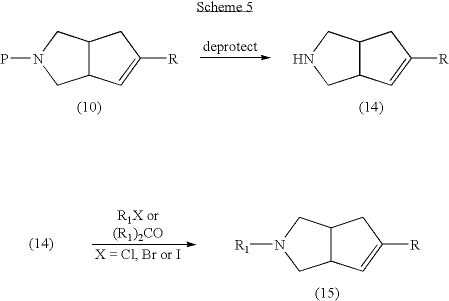

Azabicycles of general formula (14) and azabicycles of general formula (15), wherein R is aryl or heterocycle and R₁ is as defined in formula (I), may be prepared as described in Scheme 5. The nitrogen protecting group in azabicycles of general formula (10) may be removed under conditions known to those of skill in the art of organic chemistry to provide azabicycles of general formula (14). Azabicycles of general formula (14) may be treated with alkylating or acylating reagents and a base such as, but not limited to, triethylamine to provide azabicycles of general formula (15).

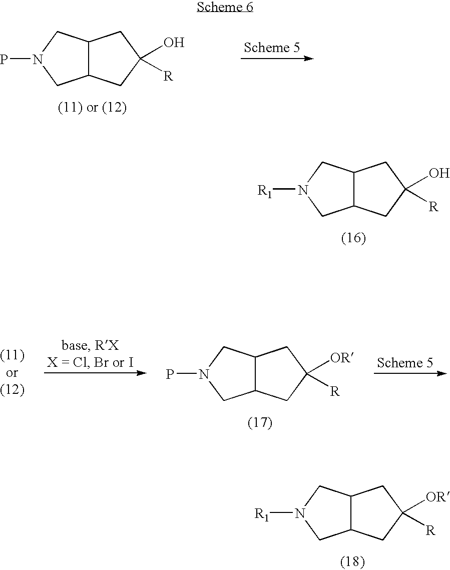

Azabicycles of general formula (16) and azabicycles of general formula (18), wherein R is aryl or heterocycle, R′ is alkyl and R₁ is as defined in formula (I), may be prepared as described in Scheme 6. Alcohols of general formula (11) or (12) may be processed as described in Scheme 5 to provide azabicycles of general formula (16). Alcohols of general formula (11) or (12) may also be treated with a base such as, but not limited to, sodium hydride and an alkylating reagent to provide alkoxy compounds of general formula (17). Alkoxy compounds of general formula (17) may be processed as described in Scheme 5 to provide azabicycles of general formula (18).

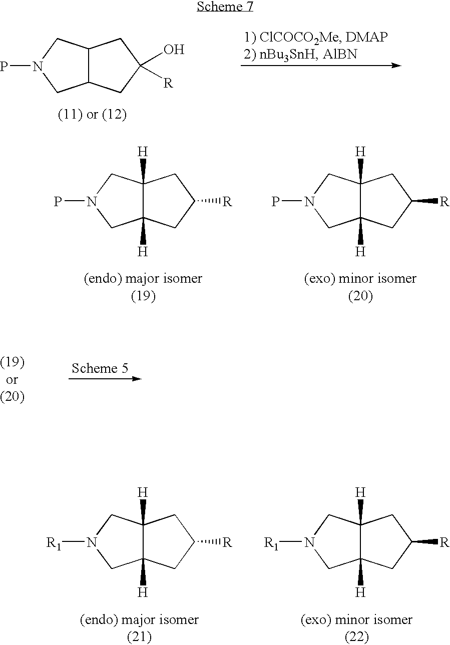

Azabicycles of general formula (21) and (22), wherein R is aryl or heterocycle and R₁ is as defined in formula (I), may be prepared as described in Scheme 7. Alcohols of general formula (11) or (12) may be deoxygenated by treatment with methyl chlorooxoacetate followed by tributyltin hydride as described in J. Org. Chem. (1996) 61(20), 7189-7191 to provide azabicyclics of general formula (19) and (20). In general, the major diastereomer produced in deoxygenation reactions performed on compounds (11) or (12) and related fused azabicyclic systems described herein is expected to be the endo-substituted isomer. Azabicyclics of general formula (19) and (20) may be processed as described in Scheme 5 to provide azabicyclics of general formula (21) and (22). The diastereomers (21) and (22) can be separated with standard chromatographic techniques used by those skilled in the art of organic chemistry.

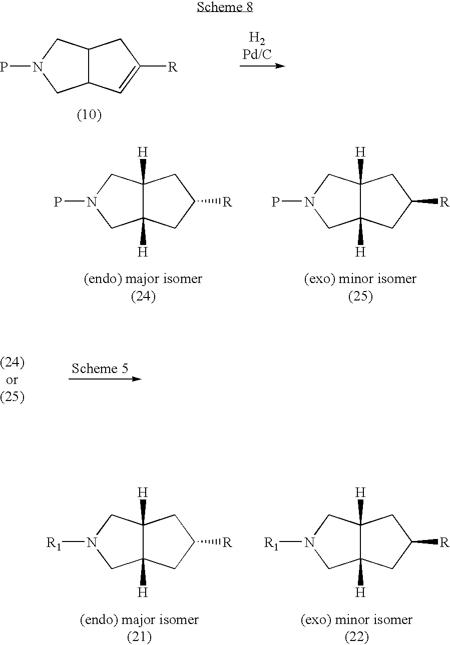

Alternatively, azabicyclics of general formula (21) and (22), wherein R is aryl or heterocycle and R₁ is as defined in formula (I), may be prepared as described in Scheme 8. Azabicycles of general formula (10) may be treated with a catalyst such as, but not limited to, palladium on carbon under an atmosphere of hydrogen to provide azabicycles of general formula (24) and (25). In general, the major diastereomer produced in hydrogenation reactions performed on compounds of general formula (10) and related azabicyclic olefins described herein is expected to be the endo-substituted isomer. Azabicycles of general formula (24) and (25) may be processed as described in Scheme 5 to provide azabicycles of general formula (21) and (22).

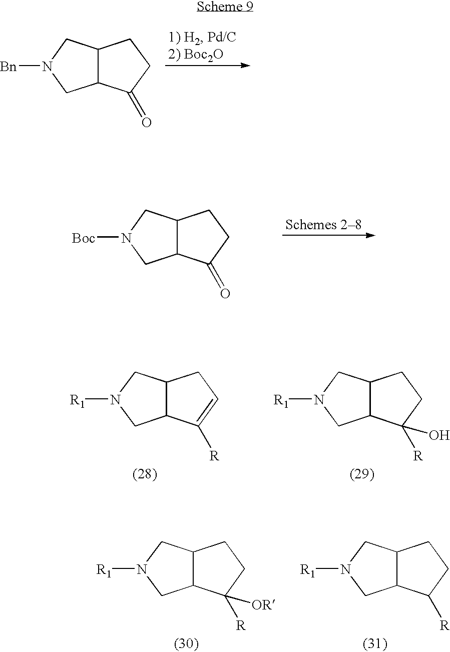

Azabicycles of general formula (28), (29), (30) and (31), wherein R is aryl or heterocycle, R′ is alkyl and R₁ is as defined in formula (I), may be prepared as described in Scheme 9. (cis)-2-Benzylhexahydrocyclopenta[c]pyrrol-4(1H)-one, prepared as described in Chem. Pharm. Bull. (1985) 33(7), 2762-2766, may be treated with a transition metal catalyst such as, but not limited to, palladium on carbon under an atmosphere of hydrogen in the presence of di-tert-butyl dicarbonate to provide tert-butyl (cis)-4-oxohexahydrocyclopenta[c]pyrrole-2(1H)-carboxylate. tert-Butyl (cis)-4-oxohexahydrocyclopenta[c]pyrrole-2(1H)-carboxylate may be processed as described in Schemes 2-8 to provide azabicycles of general formula (28), (29), (30) and (31).

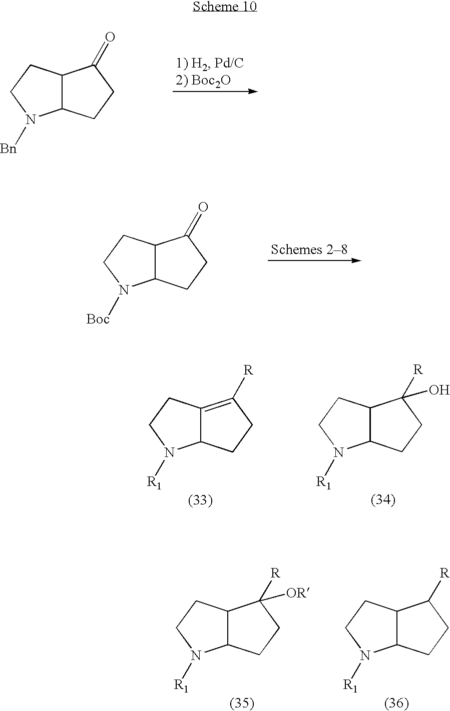

Azabicycles of general formula (33), (34), (35) and (36), wherein R is aryl or heterocycle, R′ is alkyl and R₁ is as defined in formula (I), may be prepared as described in Scheme 10. (cis)-1-Benzylhexahydrocyclopenta[b]pyrrol-4(1H)-one, prepared as described in J. Org. Chem.(1985) 50, 2403-2405, may be treated with a transition metal catalyst such as, but not limited to, palladium on carbon under an atmosphere of hydrogen in the presence of di-tert-butyl dicarbonate to provide tert-butyl (cis)-4-oxohexahydrocyclopenta[b]pyrrole-1 (2H)-carboxylate. tert-Butyl (cis)-4-oxohexahydrocyclopenta[b]pyrrole-1(2H)-carboxylate may be processed as described in Schemes 2-8 to provide azabicycles of general formula (33), (34), (35) and (36).

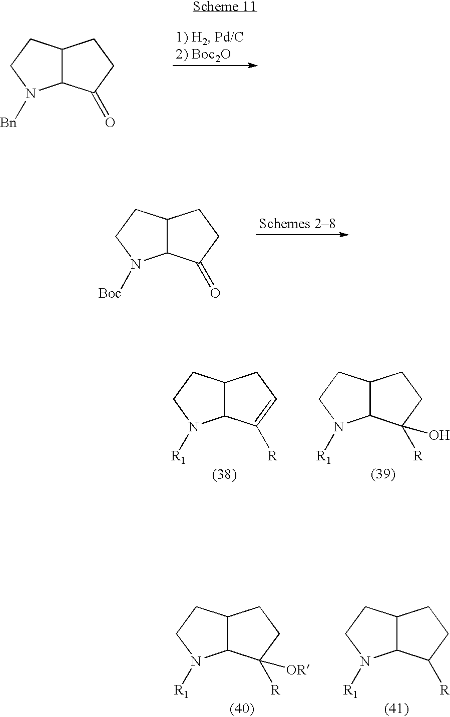

Azabicycles of general formula (38), (39), (40) and (41), wherein R is aryl or heterocycle, R′ is alkyl and R₁ is as defined in formula (I), may be prepared as described in Scheme 11. (cis)-1-Benzylhexahydrocyclopenta[b]pyrrol-6(1H)-one, prepared as described in Tetrahedron Lett. (1989) 30(41), 5547-5550, may be treated with a transition metal catalyst such as, but not limited to, palladium on carbon under an atmosphere of hydrogen in the presence of di-tert-butyl dicarbonate to provide tert-butyl (cis)-6-oxohexahydrocyclopenta[b]pyrrole-1(2H)-carboxylate. tert-Butyl (cis)-6-oxohexahydrocyclopenta[b]pyrrole-1 (2H)-carboxylate may be processed as described in Schemes 2-8 to provide azabicycles of general formula (38), (39), (40) and (41).

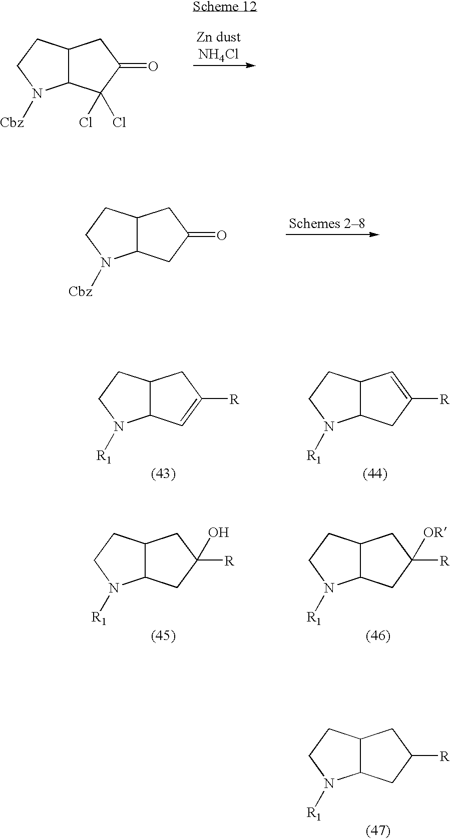

Azabicycles of general formula (43), (44), (45), (46) and (47), wherein R is aryl or heterocycle, R′ is alkyl and R₁ is as defined in formula (I), may be prepared as described in Scheme 12. Benzyl (cis)-6,6-dichloro-5-oxohexahydrocyclopenta[b]pyrrole-1(2H)-carboxylate, prepared as described in Tetrahedron Lett. (1997) 38(11), 1869-1872, may be treated with zinc dust and ammonium chloride to provide benzyl (cis)-5-oxohexahydrocyclopenta[b]pyrrole-1 (2H)-carboxylate. Benzyl (cis)-5-oxohexahydrocyclopenta[b]pyrrole-1 (2H)-carboxylate may be processed as described in Schemes 2-8 to provide azabicycles of general formula (43), (44), (45), (46) and (47).

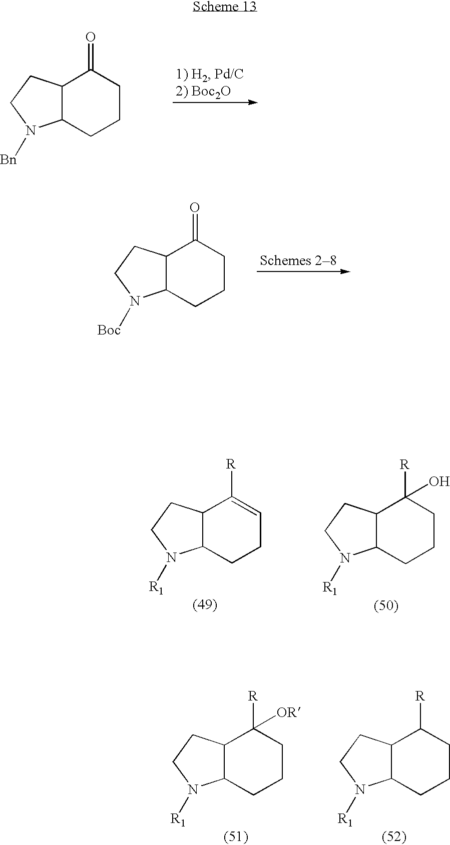

Azabicycles of general formula (49), (50), (51) and (52), wherein R is aryl or heterocycle, R′ is alkyl and R₁ is as defined in formula (I), may be prepared as described in Scheme 13. (cis)-1-Benzyloctahydro-4H-indol-4-one, prepared as described in J. Am. Chem. Soc. (1983) 105(22), 6629-6637, may be treated with a transition metal catalyst such as, but not limited to, palladium on carbon under an atmosphere of hydrogen in the presence of di-tert-butyl dicarbonate to provide tert-butyl (cis)-4-oxooctahydro-1H-indole-1-carboxylate. tert-Butyl (cis)-4-oxooctahydro-1H-indole-1-carboxylate may be processed as described in Schemes 2-8 to provide azabicycles of general formula (49), (50), (51) and (52).

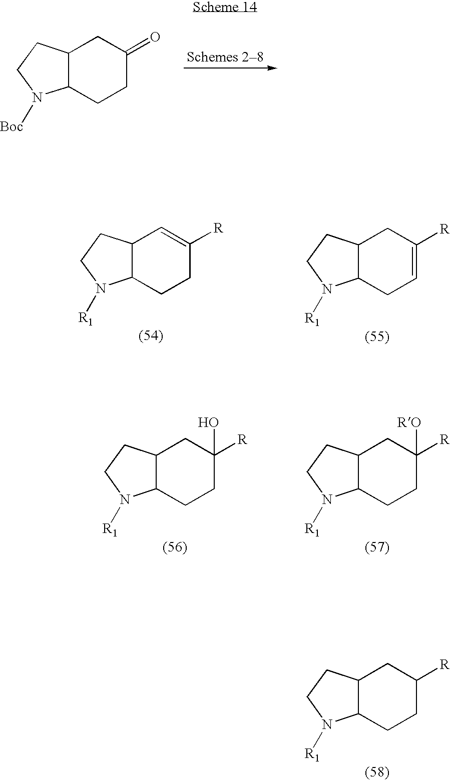

Azabicycles of general formula (54), (55), (56), (57) and (58), wherein R is aryl or heterocycle, R′ is alkyl and R₁ is as defined in formula (I), may be prepared as described in Scheme 14. tert-Butyl (cis)-5-oxooctahydro-1H-indole-1-carboxylate, prepared as described in J. Med. Chem. (1992) 35(19), 3547-3560, may be processed as described in Schemes 2-8 to provide azabicycles of general formula (54), (55), (56), (57) and (58).

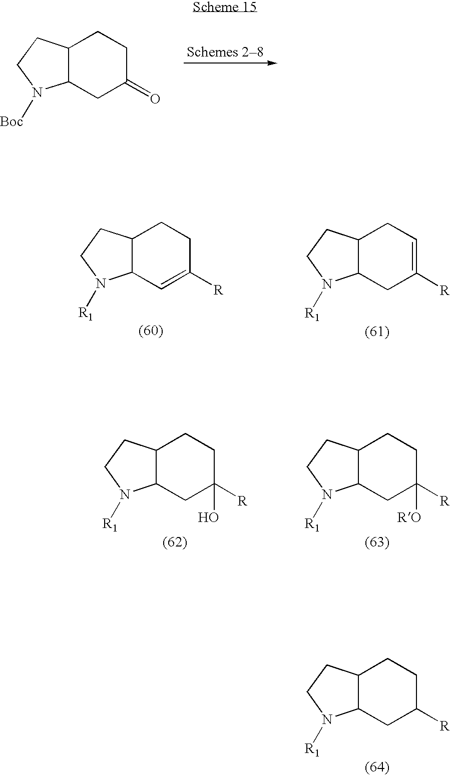

Azabicycles of general formula (60), (61), (62), (63) and (64), wherein R is aryl or heterocycle, R′ is alkyl and R₁ is as defined in formula (I), may be prepared as described in Scheme 15. tert-Butyl (cis)-6-oxooctahydro-1H-indole-1-carboxylate, prepared as described in J. Org. Chem. (1996) 61(20), 7106-7115, may be processed as described in Schemes 2-8 to provide azabicycles of general formula (60), (61), (62), (63) and (64).

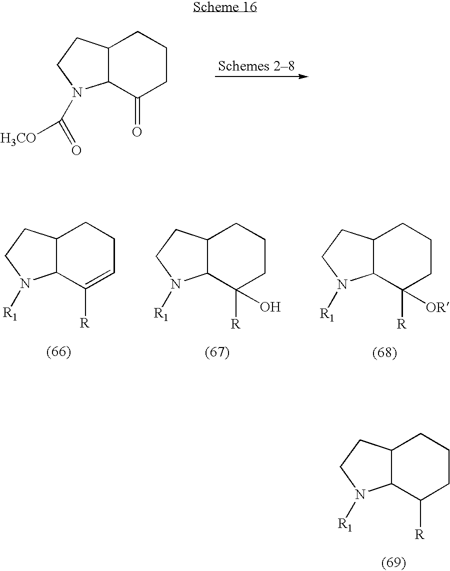

Azabicycles of general formula (66), (67), (68) and (69), wherein R is aryl or heterocycle, R′ is alkyl and R₁ is as defined in formula (I), may be prepared as described in Scheme 16. Methyl (cis)-7-oxooctahydro-1H-indole-1-carboxylate, prepared as described in J. Chem. Soc. Perkin Trans. I (1995) 2671-2672, may be processed as described in Schemes 2-8 to provide azabicycles of general formula (66), (67), (68) and (69).

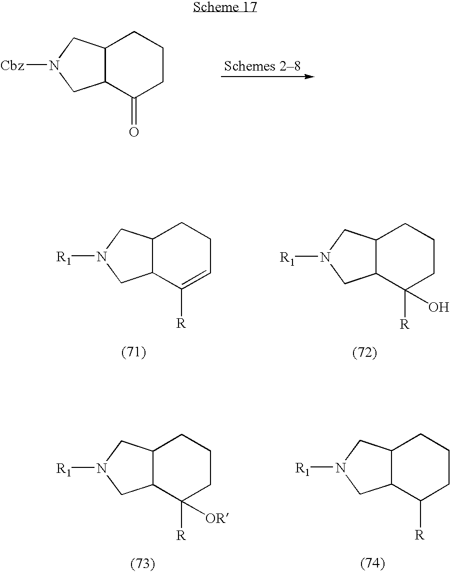

Azabicycles of general formula (71), (72), (73) and (74), wherein R is aryl or heterocycle, R′ is alkyl and R₁ is as defined in formula (I), may be prepared as described in Scheme 17. Benzyl (cis)-4-oxooctahydro-2H-isoindole-2-carboxylate, prepared as described in Eur. J. Med. Chem. (1991) 26, 889-906, may be processed as described in Schemes 2-8 to provide azabicycles of general formula (71), (72), (73) and (74).

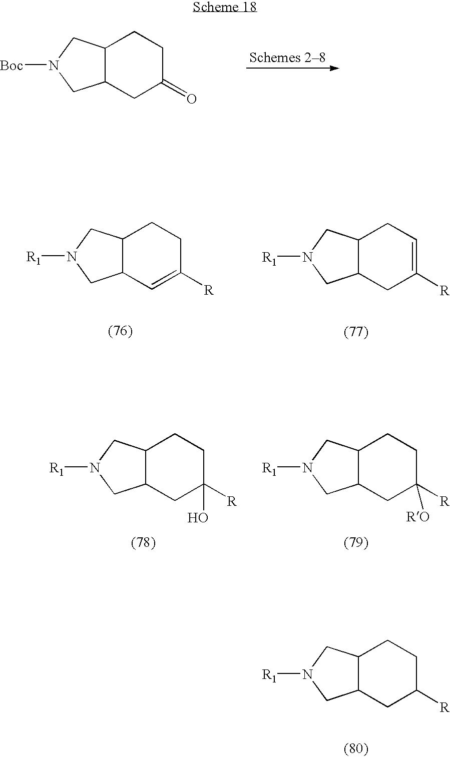

Azabicycles of general formula (76), (77), (78), (79) and (80), wherein R is aryl or heterocycle, R′ is alkyl and R₁ is as defined in formula (I), may be prepared as described in Scheme 18. tert-Butyl (cis)-5-oxooctahydro-2H-isoindole-2-carboxylate, prepared as described in WO 9806720, may be processed as described in Schemes 2-8 to provide azabicycles of general formula (76), (77), (78), (79) and (80).

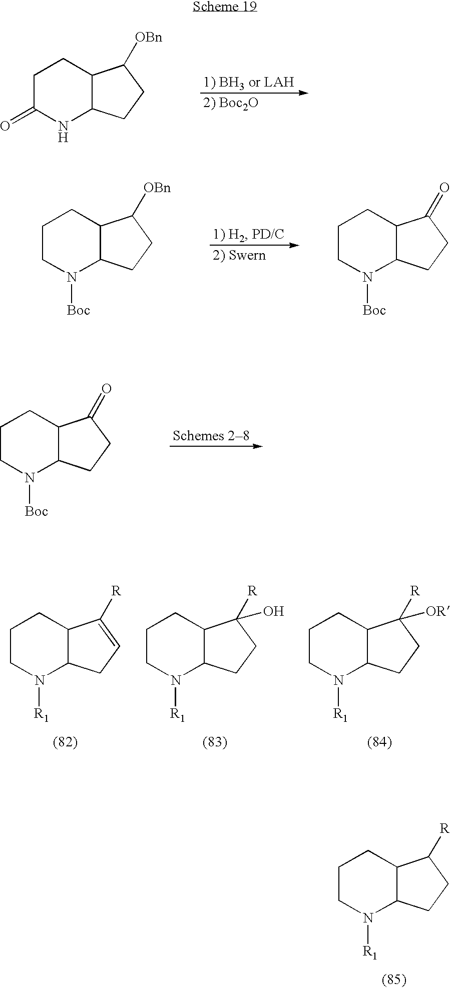

Azabicycles of general formula (82), (83), (84) and (85), wherein R is aryl or heterocycle, R′ is alkyl and R₁ is as defined in formula (I), may be prepared as described in Scheme 19. (cis)-5-(Benzyloxy)octahydro-2H-cyclopenta[b]pyridin-2-one, prepared as described in WO 9526187, may be treated with a reducing agent such as, but not limited to, borane-tetrahydrofuran complex or lithium aluminum hydride and then treated with di-tert-butyl dicarbonate (another suitable nitrogen protecting reagent may also be used such as, but not limited to, benzyl chloroformate) to provide tert-butyl (cis)-5-(benzyloxy)octahydro-1H-cyclopenta[b]pyridine-1-carboxylate. tert-Butyl (cis)-5-(benzyloxy)octahydro-1H-cyclopenta[b]pyridine-1-carboxylate may be treated with a transition metal catalyst such as, but not limited to, palladium on carbon under a hydrogen atmosphere and then treated with an oxidizing agent to provide tert-butyl (cis)-5-oxooctahydro-1H-cyclopenta[b]pyridine-1-carboxylate. tert-Butyl (cis)-5-oxooctahydro-1H-cyclopenta[b]pyridine-1-carboxylate may be processed as described in Schemes 2-8 to provide azabicycles of general formula (82), (83), (84) and (85).

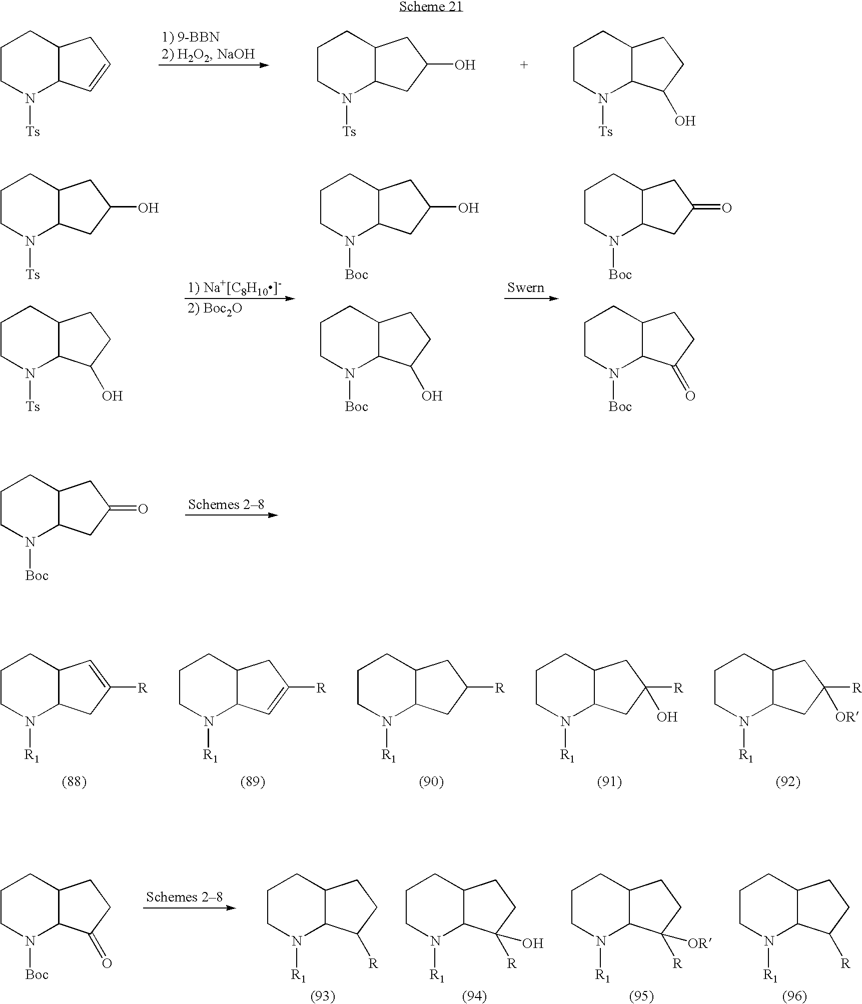

Azabicycles of general formula (88), (89), (90), (91), (92), (93), (94), (95) and (96), wherein R is aryl or heterocycle, R′ is alkyl and R₁ is as defined in formula (I), may be prepared as described in Scheme 21. (cis)-1-[(4-Methylphenyl)sulfonyl]-2,3,4,4a,5,7a-hexahydro-1H-cyclopenta[b]pyridine, prepared as described in J. Org. Chem. (1996) 61(11), 3584-3585, may be treated with a hydroborating reagent such as, but not limited to, diborane or 9-borabicyclo[3.3.1]nonane followed by treatment with hydrogen peroxide and aqueous sodium hydroxide to provide stereoisomeric alcohols, (cis)-1-[(4-methylphenyl)sulfonyl]octahydro-1H-cyclopenta[b]pyridin-6-ol and (cis)-1-[(4-methylphenyl)sulfonyl]octahydro-1H-cyclopenta[b]pyridin-7-ol. The alcohols may be separated using chromatographic methods known to those of skill in the art of organic chemistry. The alcohols may be treated with sodium naphthalenide and then treated with di-tert-butyl dicarbonate to provide tert-butyl (cis)-6-hydroxyoctahydro-1H-cyclopenta[b]pyridine-1-carboxylate or tert-butyl (cis)-7-hydroxyoctahydro-1H-cyclopenta[b]pyridine-1-carboxylate. The alcohols may be oxidized under Swern conditions (DMSO/oxalyl chloride/triethylamine) to provide the corresponding ketones, tert-butyl (cis)-6-oxooctahydro-1H-cyclopenta[b]pyridine-1-carboxylate or tert-butyl (cis)-7-oxooctahydro-1H-cyclopenta[b]pyridine-1-carboxylate. tert-Butyl (cis)-6-oxooctahydro-1H-cyclopenta[b]pyridine-1-carboxylate may be processed as described in Schemes 2-8 to provide azabicycles of general formula (88), (89), (90), (91) and (92). tert-Butyl (cis)-7-oxooctahydro-1H-cyclopenta[b]pyridine-1-carboxylate may be processed as described in Schemes 2-8 to provide azabicycles of general formula (93), (94), (95) and (96).

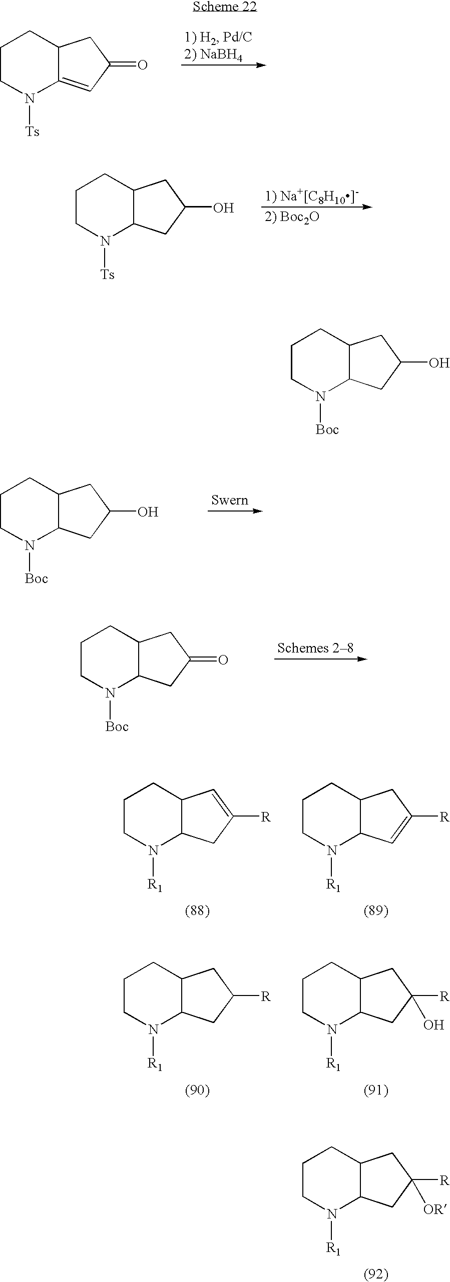

An alternative method of preparing azabicycles of general formula (88), (89), (90), (91) and (92), wherein R is aryl or heterocycle, R′ is alkyl and R₁ is as defined in formula (I), may be used as described in Scheme 22. 1-[(4-Methylphenyl)sulfonyl]-1,2,3,4,4a,5-hexahydro-6H-cyclopenta[b]pyridin-6-one, prepared as described in J. Org. Chem. (1996) 61(16), 5540-5552, may be treated with a transition metal catalyst such as, but not limited to, palladium on carbon under a hydrogen atmosphere and then treated with a reducing agent such as, but not limited to, sodium borohydride to provide stereoisomeric alcohols. The stereoisomeric alcohols may be treated with sodium naphthalenide and then treated with di-tert-butyl dicarbonate to provide the N-boc protected stereoisomeric alcohols. The N-boc protected stereoisomeric alcohols may be oxidized under Swern conditions (DMSO/oxalyl chloride/triethylamine) to provide tert-butyl (cis)-6-oxooctahydro-1H-cyclopenta[b]pyridine-1-carboxylate. tert-Butyl (cis)-6-oxooctahydro-1H-cyclopenta[b]pyridine-1-carboxylate may be processed as described in Schemes 2-8 to provide azabicycles of general formula (88), (89), (90), (91) and (92).

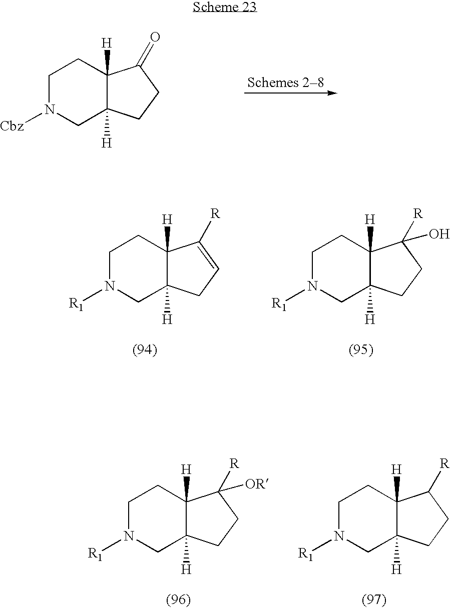

Azabicycles of general formula (94), (95), (96) and (97), wherein R is aryl or heterocycle, R′ is alkyl and R₁ is as defined in formula (I), may be prepared as described in Scheme 23. Benzyl (trans)-5-oxooctahydro-2H-cyclopenta[c]pyridine-2-carboxylate, prepared as described in Eur. J. Med. Chem. (1991) 26, 889-906, may be processed as described in Schemes 2-8 to provide azabicycles of general formula (94), (95), (96) and (97).

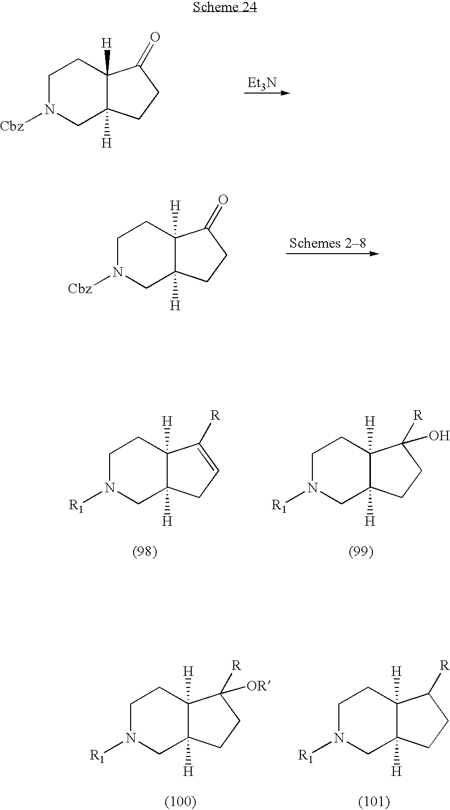

Azabicycles of general formula (98), (99), (100) and (101), wherein R is aryl or heterocycle, R′ is alkyl and R₁ is as defined in formula (I), may be prepared as described in Scheme 24. Benzyl (trans)-5-oxooctahydro-2H-cyclopenta[c]pyridine-2-carboxylate, prepared as described in Eur. J. Med. Chem. (1991) 26, 889-906, may be epimerized with a base such as, but not limited to, triethylamine to provide benzyl (cis)-5-oxooctahydro-2H-cyclopenta[c]pyridine-2-carboxylate. Benzyl (cis)-5-oxooctahydro-2H-cyclopenta[c]pyridine-2-carboxylate may be processed as described in Schemes 2-8 to provide azabicycles of general formula (98), (99), (100) and (101).

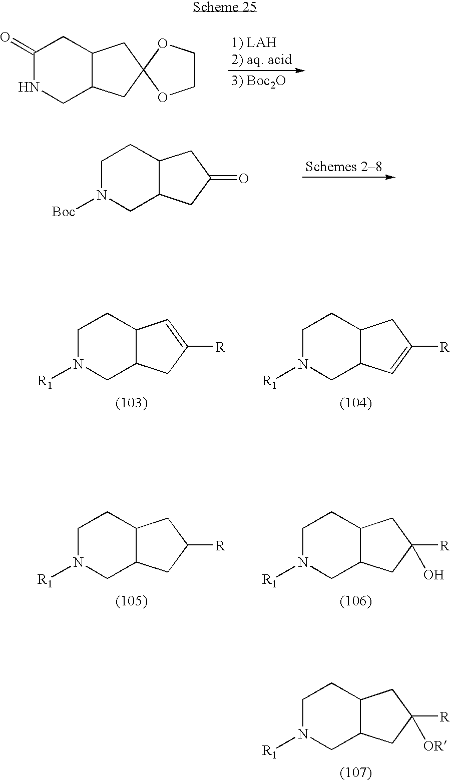

Azabicycles of general formula (103), (104), (105), (106) and (107), wherein R is aryl or heterocycle, R′ is alkyl and R₁ is as defined in formula (I), may be prepared as described in Scheme 25. (cis)-Hexahydro-spiro[6H-cyclopenta[c]pyridine-6,2′-[1,3]dioxolan]-3(2H)-one, prepared as described in Tetrahedron: Asymmetry (1997) 8(17), 2893-2903 maybe treated with a reducing agent such as, but not limited to, borane tetrahydrofuran complex or lithium aluminum hydride, treated with aqueous acid and then treated with di-tert-butyl dicarbonate to provide tert-butyl (cis)-6-oxooctahydro-2H-cyclopenta[c]pyridine-2-carboxylate. tert-Butyl (cis)-6-oxooctahydro-2H-cyclopenta[c]pyridine-2-carboxylate may be processed as described in Schemes 2-8 to provide azabicycles of general formula (103), (104), (105), (106) and (107).

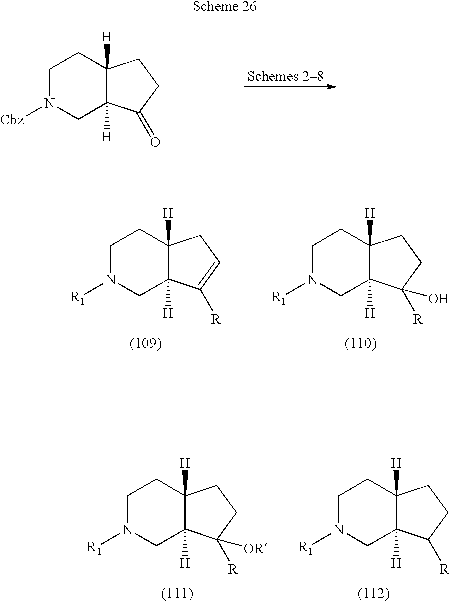

Azabicycles of general formula (109), (110), (111) and (112), wherein R is aryl or heterocycle, R′ is alkyl and R₁ is as defined in formula (I), may be prepared as described in Scheme 26. Benzyl (trans)-7-oxooctahydro-2H-cyclopenta[c]pyridine-2-carboxylate, prepared as described in Eur. J. Med. Chem. (1991) 26, 889-906, may be processed as described in Schemes 2-8 to provide azabicycles of general formula (109), (110), (111) and (112).

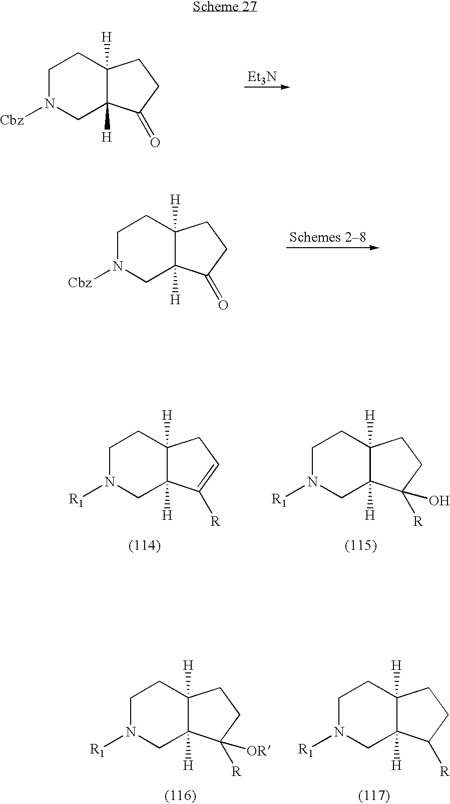

Azabicycles of general formula (114), (115), (116) and (117), wherein R is aryl or heterocycle, R′ is alkyl and R₁ is as defined in formula (I), may be prepared as described in Scheme 27. Benzyl (trans)-7-oxooctahydro-2H-cyclopenta[c]pyridine-2-carboxylate, prepared as described in Eur. J. Med. Chem. (1991) 26, 889-906, may be epimerized with a base such as, but not limited to, triethylamine to provide benzyl (cis)-7-oxooctahydro-2H-cyclopenta[c]pyridine-2-carboxylate. Benzyl (cis)-7-oxooctahydro-2H-cyclopenta[c]pyridine-2-carboxylate may be processed as described in Schemes 2-8 to provide azabicycles of general formula (114), (115), (116) and (117).

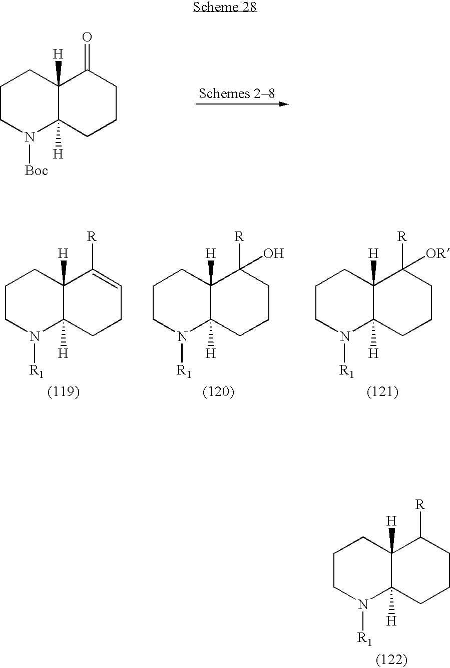

Azabicycles of general formula (119), (120), (121) and (122), wherein R is aryl or heterocycle, R′ is alkyl and R₁ is as defined in formula (I), may be prepared as described in Scheme 28. tert-Butyl (trans)-5-oxooctahydro-1(2H)-quinolinecarboxylate, prepared as described in J. Med. Chem. (1991) 34(9), 2736-2746, may be processed as described in Schemes 2-8 to provide azabicycles of general formula (119), (120), (121) and (122).

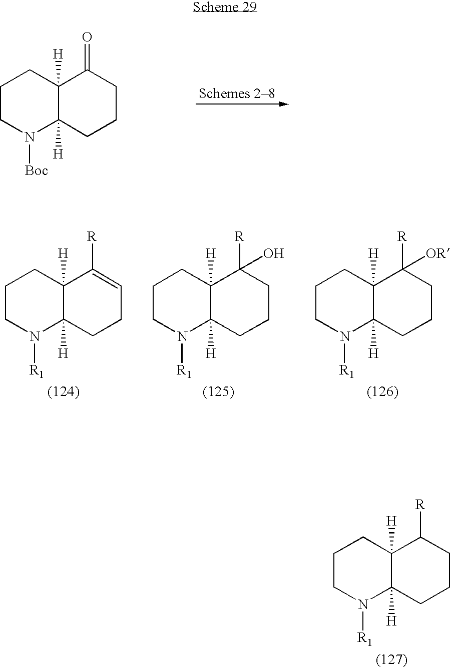

Azabicycles of general formula (124), (125), (126) and (127), wherein R is aryl or heterocycle, R′ is alkyl and R₁ is as defined in formula (I), may be prepared as described in Scheme 29. tert-Butyl (cis)-5-oxooctahydro-1(2H)-quinolinecarboxylate, prepared as described in J. Med. Chem. (1991) 34(9), 2736-2746, may be processed as described in Schemes 2-8 to provide azabicycles of general formula (124), (125), (126) and (127).

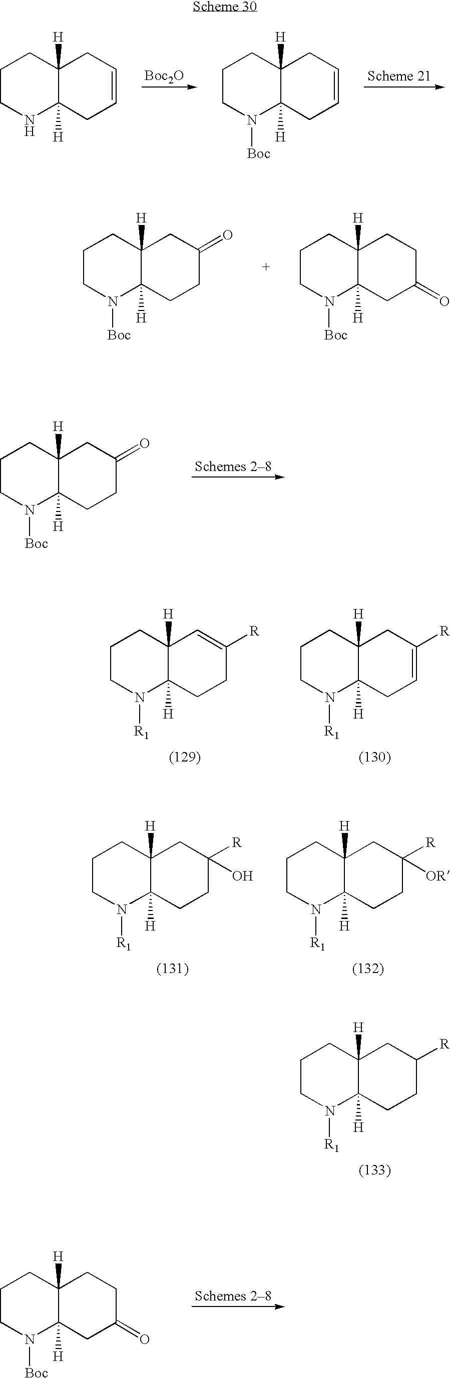

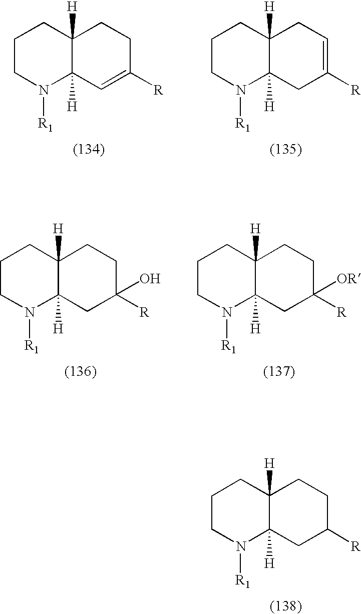

Azabicycles of general formula (129), (130), (131), (132), (133), (134), (135), (136), (137) and (138), wherein R is aryl or heterocycle, R′ is alkyl and R₁ is as defined in formula (I), may be prepared as described in Scheme 30. (trans)-1,2,3,4,4a,5,8,8a-Octahydroquinoline, prepared as described in J. Med. Chem. (1999) 42(15), 2862-2869, may be treated with di-tert-butyl dicarbonate in a solvent such as, but not limited to, THF or 1,4-dioxane to provide tert-butyl (trans)-3,4,4a,5,8,8a-hexahydro-1(2H)-quinolinecarboxylate. tert-Butyl (trans)-3,4,4a,5,8,8a-hexahydro-1(2H)-quinolinecarboxylate may be processed as described in Scheme 21 to provide ketones tert-butyl (trans)-6-oxooctahydro-1(2H)-quinolinecarboxylate and tert-butyl (trans)-7-oxooctahydro-1(2H)-quinolinecarboxylate. The ketones may be separated using methods known to those of skill in the art of organic chemistry such as, but not limited to, chromatography. tert-Butyl (trans)-6-oxooctahydro-1 (2H)-quinolinecarboxylate may be processed as described in Schemes 2-8 to provide azabicycles of general formula (129), (130), (131), (132) and (133). tert-Butyl (trans)-7-oxooctahydro-1(2H)-quinolinecarboxylate maybe processed as described in Schemes 2-8 to provide azabicycles of general formula (134), (135), (136), (137) and (138).

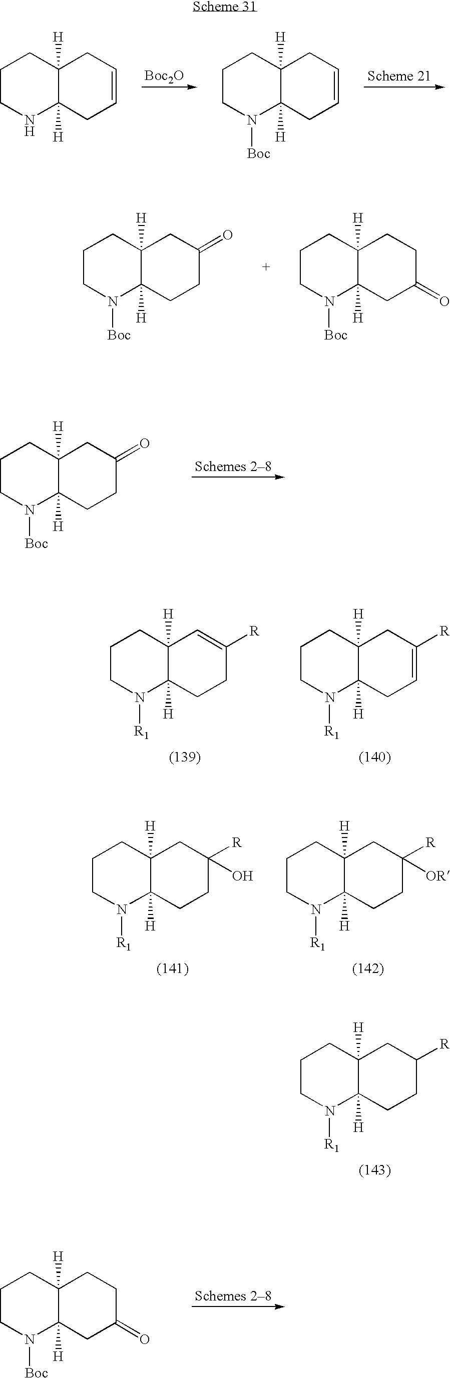

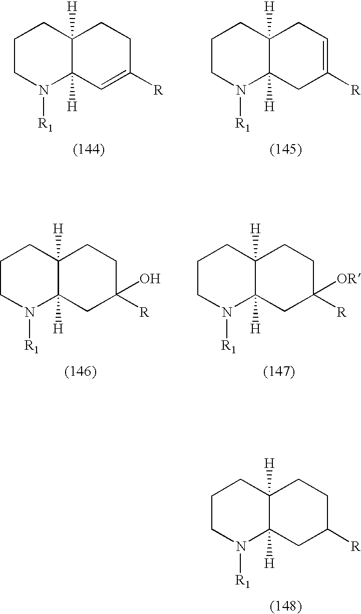

Azabicycles of general formula (139), (140), (141), (142), (143), (144), (145), (146), (147) and (148), wherein R is aryl or heterocycle, R′ is alkyl and R₁ is as defined in formula (I), maybe prepared as described in Scheme 31. (cis)-1,2,3,4,4a,5,8,8a-Octahydroquinoline, prepared as described in J. Med. Chem. (1999) 42(15), 2862-2869, may be treated with di-tert-butyl dicarbonate in a solvent such as, but not limited to, THF or 1,4-dioxane to provide tert-butyl (cis)-3,4,4a,5,8,8a-hexahydro-1 (2H)-quinolinecarboxylate. tert-Butyl (cis)-3,4,4a,5,8,8a-hexahydro-1 (2H)-quinolinecarboxylate may be processed as described in Scheme 21 to provide ketones tert-butyl (cis)-6-oxooctahydro-1 (2H)-quinolinecarboxylate and tert-butyl (cis)-7-oxooctahydro-1 (2H)-quinolinecarboxylate. The ketones may be separated using methods known to those of skill in the art of organic chemistry such as, but not limited to, chromatography. tert-Butyl (cis)-6-oxooctahydro-1 (2H)-quinolinecarboxylate may be processed as described in Schemes 2-8 to provide azabicycles of general formula (139), (140), (141), (142) and (143). tert-Butyl (cis)-7-oxooctahydro-1(2H)-quinolinecarboxylate may be processed as described in Schemes 2-8 to provide azabicycles of general formula (144), (145), (146), (147) and (148).

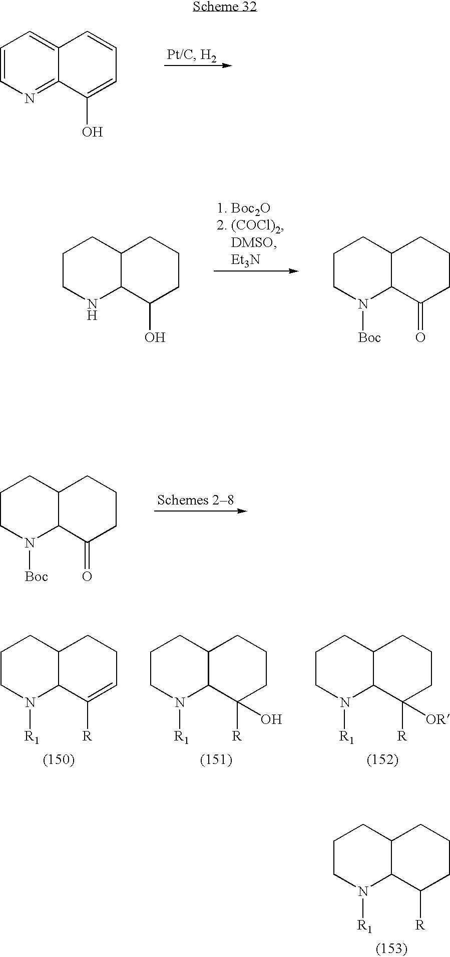

Azabicycles of general formula (150), (151), (152) and (153), wherein R is aryl or heterocycle, R′ is alkyl and R₁ is as defined in formula (I), may be prepared as described in Scheme 32. 8-Quinolinol, purchased from Aldrich, may be treated with a platinum catalyst such as, but not limited to, platinum on carbon under a hydrogen atmosphere to provide (cis)-decahydro-8-quinolinol. (cis)-Decahydro-8-quinolinol may be treated with a nitrogen protecting group reagent such as, but not limited to, di-tert-butyl dicarbonate and then oxidized under Swern conditions to provided tert-butyl (cis)-8-oxooctahydro-1(2H)-quinolinecarboxylate. tert-Butyl (cis)-8-oxooctahydro-1(2H)-quinolinecarboxylate may be processed as described in Schemes 2-8 to provide azabicycles of general formula (150), (151), (152) and (153).

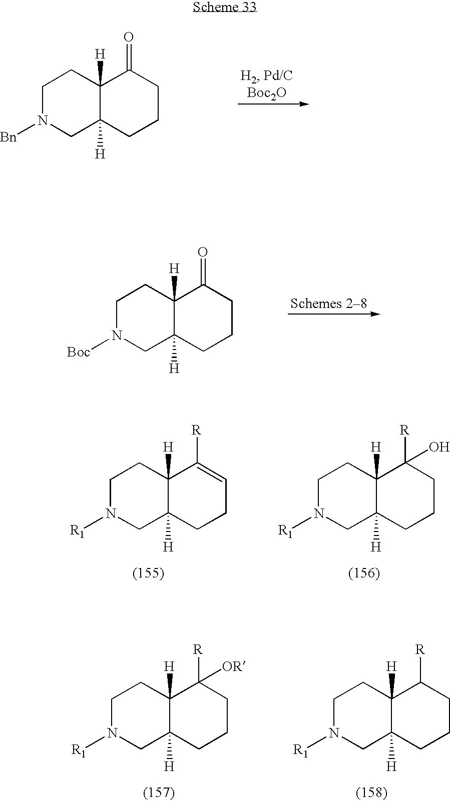

Azabicycles of general formula (155), (156), (157) and (158), wherein R is aryl or heterocycle, R′ is alkyl and R₁ is as defined in formula (I), may be prepared as described in Scheme 33. (trans)-2-Benzyloctahydro-5(1H)-isoquinolinone, prepared as described in J. Am. Chem. Soc. (1991) 113(23), 8863-8878, may be treated with a metal catalyst such as, but not limited to, palladium on carbon under a hydrogen atmosphere in the presence of di-tert-butyl dicarbonate to provide tert-butyl (trans)-5-oxooctahydro-2(1H)-isoquinolinecarboxylate. tert-Butyl (trans)-5-oxooctahydro-2(1H)-isoquinolinecarboxylate may be processed as described in Schemes 2-8 to provide azabicycles of general formula (155), (156), (157) and (158).

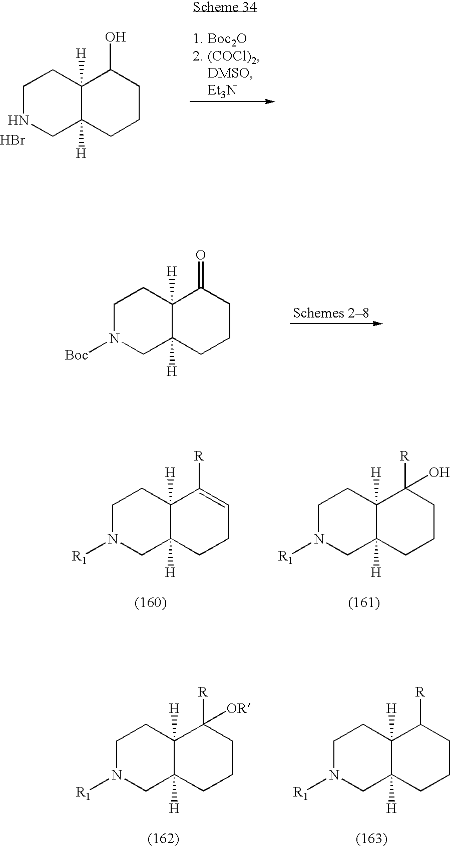

Azabicycles of general formula (160), (161), (162) and (163), wherein R is aryl or heterocycle, R′ is alkyl and R₁ is as defined in formula (I), may be prepared as described in Scheme 34. (cis)-Decahydro-5-isoquinolinol hydrobromide, prepared as described in J. Med. Chem. (1968) 11(5), 997-100, may be treated with di-tert-butyl dicarbonate and then oxidized under Swern conditions to provide tert-butyl (cis)-5-oxooctahydro-2(1H)-isoquinolinecarboxylate. tert-Butyl (cis)-5-oxooctahydro-2(1H)-isoquinolinecarboxylate may be processed as described in Schemes 2-8 to provide azabicycles of general formula (160), (161), (162) and (163).

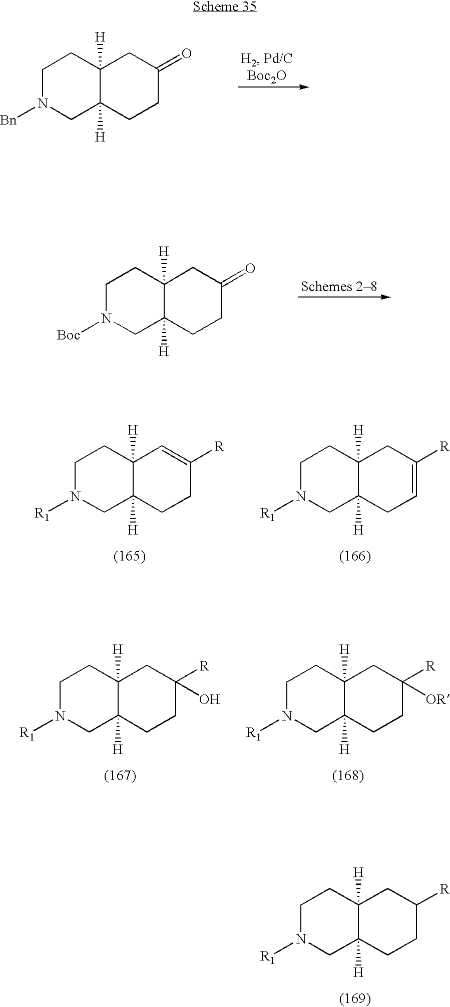

Azabicycles of general formula (165), (166), (167), (168) and (169), wherein R is aryl or heterocycle, R′ is alkyl and R₁ is as defined in formula (I), may be prepared as described in Scheme 35. (cis)-2-Benzyloctahydro-6(2H)-isoquinolinone, prepared as described in J. Med. Chem. (1987) 30(7), 1210-1214, may be treated with a transition metal catalyst such as, but not limited to, palladium on carbon under an atmosphere of hydrogen in the presence of di-tert-butyl dicarbonate to provide tert-butyl (cis)-6-oxooctahydro-2(1H)-isoquinolinecarboxylate. tert-Butyl (cis)-6-oxooctahydro-2(1H)-isoquinolinecarboxylate may be processed as described in Schemes 2-8 to provide azabicycles of general formula (165), (166), (167), (168) and (169).

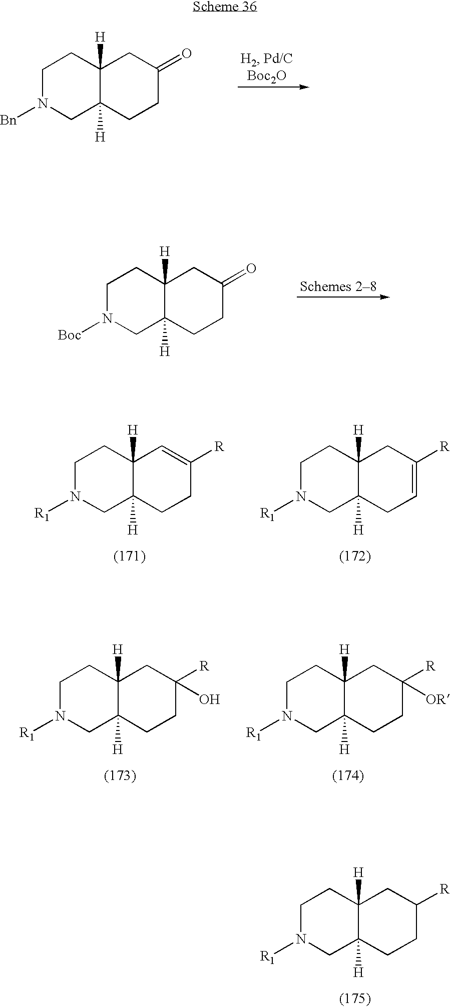

Azabicycles of general formula (171), (172), (173), (174) and (175), wherein R is aryl or heterocycle, R′ is alkyl and R₁ is as defined in formula (I), may be prepared as described in Scheme 36. (trans)-2-Benzyloctahydro-6(2H)-isoquinolinone, prepared as described in J. Med. Chem. (1987) 30(7), 1210-1214, may be treated with a transition metal catalyst such as, but not limited to, palladium on carbon under an atmosphere of hydrogen in the presence of di-tert-butyl dicarbonate to provide tert-butyl (trans)-6-oxooctahydro-2(1H)-isoquinolinecarboxylate. tert-Butyl (trans)-6-oxooctahydro-2(1H)-isoquinolinecarboxylate may be processed as described in Schemes 2-8 to provide azabicycles of general formula (171), (172), (173), (174) and (175).

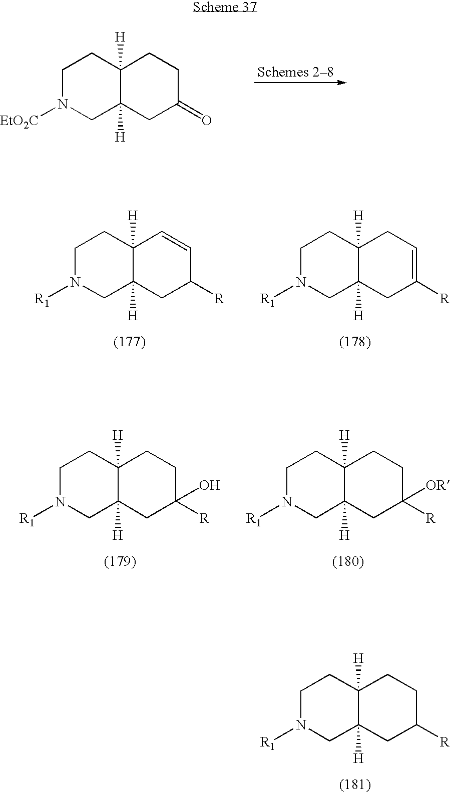

Azabicycles of general formula (177), (178), (179), (180) and (181), wherein R is aryl or heterocycle, R′ is alkyl and R₁ is as defined in formula (I), may be prepared as described in Scheme 37. Ethyl (cis)-7-oxooctahydro-2(1H)-isoquinolinecarboxylate, prepared as described in J. Am. Chem. Soc. (1998) 120(41), 10676-10686, may be processed as described in Schemes 2-8 to provide azabicycles of general formula (177), (178), (179), (180) and (181).

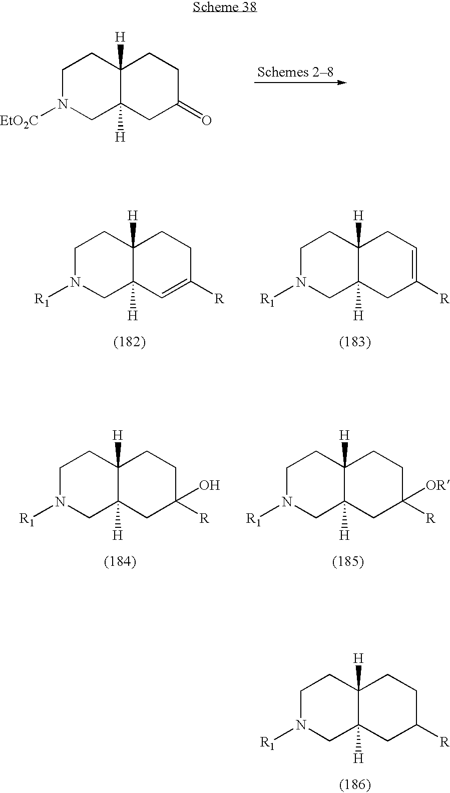

Azabicycles of general formula (182), (183), (184), (185) and (186), wherein R is aryl or heterocycle, R′ is alkyl and R₁ is as defined in formula (I), may be prepared as described in Scheme 38. Ethyl (trans)-7-oxooctahydro-2(1H)-isoquinolinecarboxylate, prepared as described in J. Am. Chem. Soc. (1998) 120(41), 10676-10686, may be processed as described in Schemes 2-8 to provide azabicycles of general formula (182), (183), (184), (185) and (186).

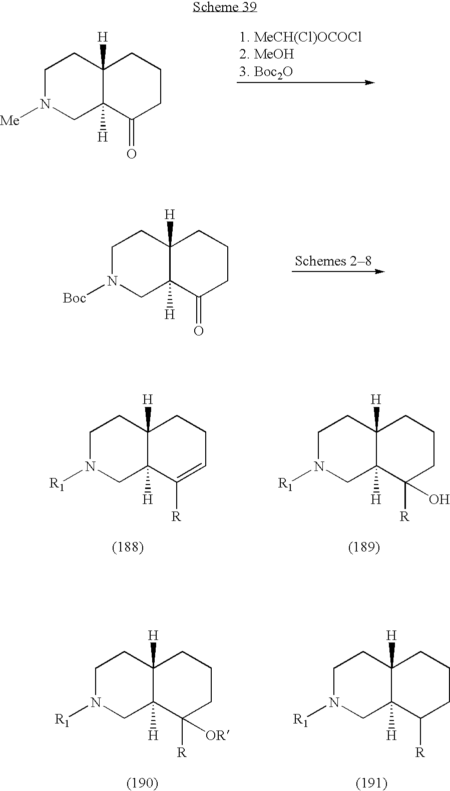

Azabicycles of general formula (188), (189), (190) and (191), wherein R is aryl or heterocycle, R′ is alkyl and R₁ is as defined in formula (I), may be prepared as described in Scheme 39. (trans)-2-Methyloctahydro-8(2H)-isoquinolinone, prepared as described in J. Org. Chem. (1974) 39(22), 3210-3215, may be treated with 1-chloroethyl chloroformate followed by methanol and then treatment with di-tert-butyl dicarbonate to provide tert-butyl (trans)-8-oxooctahydro-2(1H)-isoquinolinecarboxylate. tert-Butyl (trans)-8-oxooctahydro-2(1H)-isoquinolinecarboxylate may be processed as described in Schemes 2-8 to provide azabicycles of general formula (188), (189), (190) and (191).

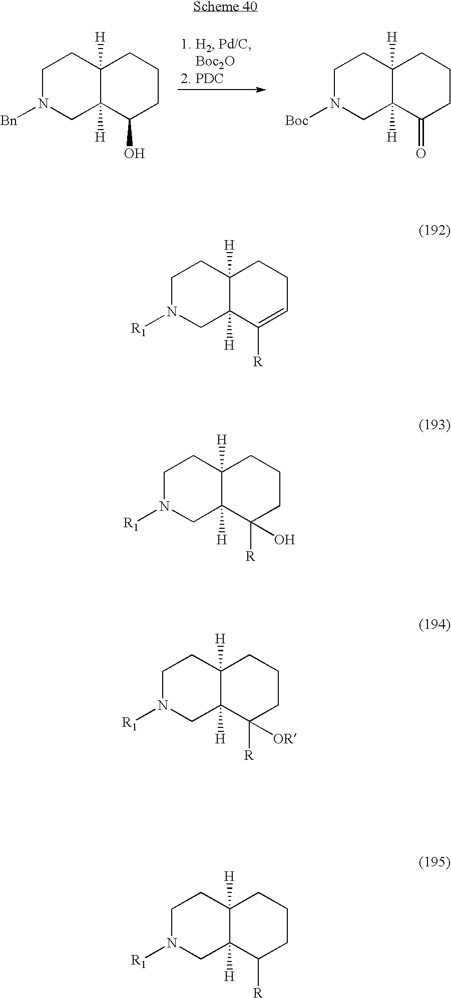

Azabicycles of general formula (192), (193), (194) and (195), wherein R is aryl or heterocycle, R′ is alkyl and R₁ is as defined in formula (I), may be prepared as described in Scheme 40. (cis)-2-Benzyldecahydro-8-isoquinolinol, prepared as described in Tetrahedron Lett. (1998) 39(29), 5185-5188, may be treated with a metal catalyst such as, but not limited to, palladium on carbon under a hydrogen atmosphere in the presence of di-tert-butyl dicarbonate and then oxidized with pyridinium dichromate (PDC) to provide tert-butyl (cis)-8-oxooctahydro-2(1H)-isoquinolinecarboxylate. tert-Butyl (cis)-8-oxooctahydro-2(1H)-isoquinolinecarboxylate may be processed as described in Schemes 2-8 to provide azabicycles of general formula (192), (193), (194) and (195).

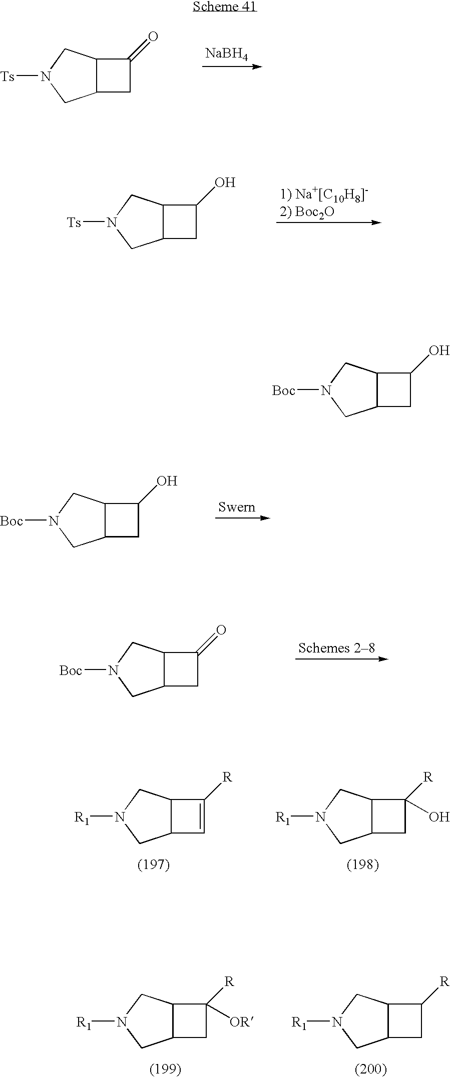

Azabicycles of general formula (197), (198), (199) and (200), wherein R is aryl or heterocycle, R′ is alkyl and R₁ is as defined in formula (I), may be prepared as described in Scheme 41. (cis)-3-[(4-Methylphenyl)sulfonyl]-3-azabicyclo[3.2.0]heptan-6-one, prepared as described in Heterocycles (1989) 28(1), 29-32, may be treated with a reducing agent such as, but not limited to, sodium borohydride to provide stereoisomeric alcohols. The stereoisomeric alcohols may be treated with sodium naphthalenide and then treated with di-tert-butyl dicarbonate to provide N-boc protected stereoisomeric alchols. The N-boc protected stereoisomeric alcohols may be oxidized agent under Swern conditions (DMSO/oxalyl chloride/triethylamine) to provide tert-butyl (cis)-6-oxo-3-azabicyclo[3.2.0]heptane-3-carboxylate. tert-Butyl (cis)-6-oxo-3-azabicyclo[3.2.0]heptane-3-carboxylate may be processed as described in Schemes 2-8 to provide azabicycles of general formula (197), (198), (199) and (200).

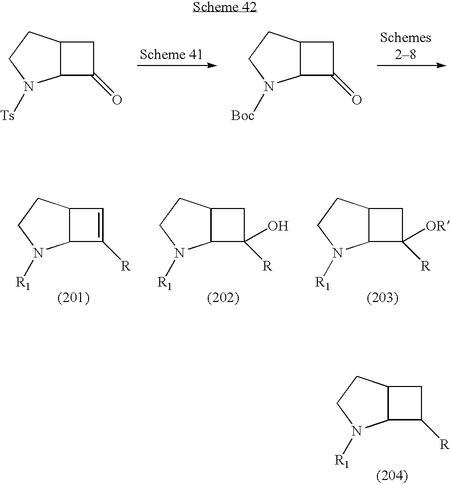

Azabicycles of general formula (201), (202), (203) and (204), wherein R is aryl or heterocycle, R′ is alkyl and R₁ is as defined in formula (I), may be prepared as described in Scheme 42. (cis)-2-[(4-Methylphenyl)sulfonyl]-2-azabicyclo[3.2.0]heptan-7-one, prepared as described in Heterocycles (1989) 28(1), 29-32, may be processed as described in Scheme 41 to provide tert-butyl (cis)-7-oxo-2-azabicyclo[3.2.0]heptane-2-carboxylate. tert-Butyl (cis)-7-oxo-2-azabicyclo[3.2.0]heptane-2-carboxylate may be processed as described in Schemes 2-8 to provide azabicycles of general formula (201), (202), (203) and (204).

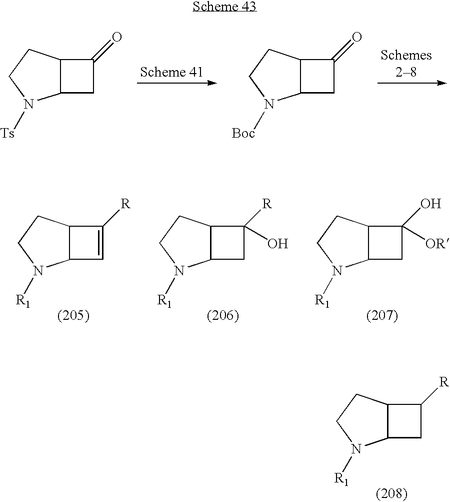

Azabicycles of general formula (205), (206), (207) and (208), wherein R is aryl or heterocycle, R′ is alkyl and R₁ is as defined in formula (I), may be prepared as described in Scheme 43. (cis)-2-[(4-Methylphenyl)sulfonyl]-2-azabicyclo[3.2.0]heptan-6-one, prepared as described in Tetrahedron Lett. (1993) 34(1), 27-30, may be processed as described in Scheme 41 to provide tert-butyl (cis)-6-oxo-2-azabicyclo[3.2.0]heptane-2-carboxylate. tert-Butyl (cis)-6-oxo-2-azabicyclo[3.2.0]heptane-2-carboxylate may be processed as described in Schemes 2-8 to provide azabicycles of general formula (205), (206), (207) and (208).

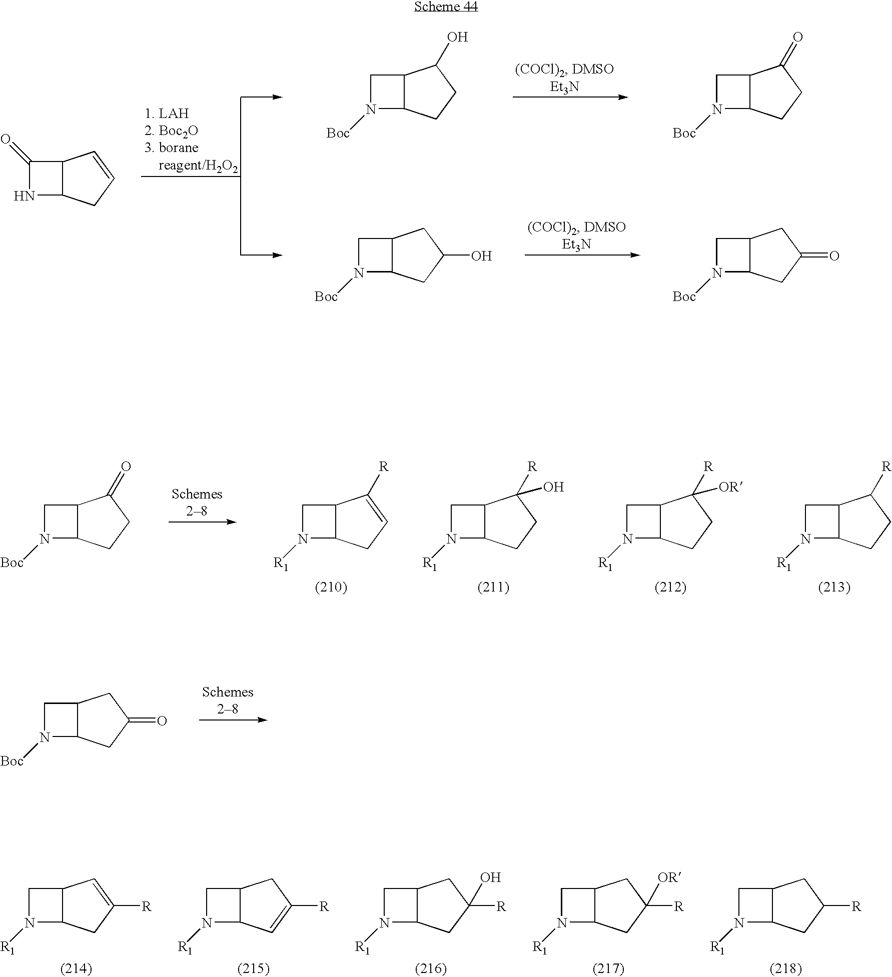

Azabicycles of general formula (210), (211), (212), (213), (214), (215), (216), (217) and (218), wherein R is aryl or heterocycle, R′ is alkyl and R₁ is as defined in formula (I), may be prepared as described in Scheme 44. (cis)-6-Azabicyclo[3.2.0]hept-2-en-7-one, prepared as described in Tetrahedron Lett. (1999) 40(15), 4857-4860, may be treated with lithium aluminum hydride (LAH) followed by di-tert-butyl dicarbonate and then treatment with a hydroborating agent such as, but not limited to, 9-borabicyclo[3.3.1]nonane to provide a stereoisomeric mixture of alcohols, tert-butyl (cis)-2-hydroxy-6-azabicyclo[3.2.0]heptane-6-carboxylate and tert-butyl (cis)-3-hydroxy-6-azabicyclo[3.2.0]heptane-6-carboxylate. The alcohols may be separated using methods known to those of skill in the art of organic chemistry such as, but not limited to, chromatography and then oxidized under Swern conditions to provide the corresponding ketones. tert-Butyl (cis)-2-oxo-6-azabicyclo[3.2.0]heptane-6-carboxylate may be processed as described in Schemes 2-8 to provide azabicycles of general formula (210), (211), (212) and (213). tert-Butyl (cis)-3-oxo-6-azabicyclo[3.2.0]heptane-6-carboxylate may be processed as described in Schemes 2-8 to provide azabicycles of general formula (214), (215), (216), (217) and (218).

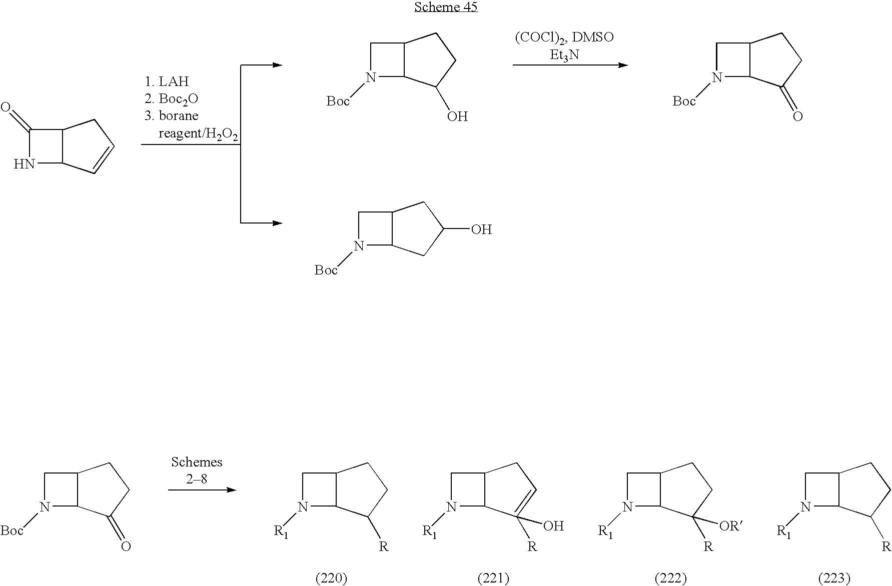

Azabicycles of general formula (220), (221), (222) and (223), wherein R is aryl or heterocycle, R′ is alkyl and R₁ is as defined in formula (I), may be prepared as described in Scheme 45. (cis)-6-Azabicyclo[3.2.0]hept-3-en-7-one, prepared as described in Tetrahedron Lett. (1985) 26(16), 1907-1910, may be treated with lithium aluminum hydride (LAH) followed by di-tert-butyl dicarbonate and then treatment with a hydroborating agent such as, but not limited to, 9-borabicyclo[3.3.1]nonane to provide a stereoisomeric mixture of alcohols, tert-butyl (cis)-3-hydroxy-6-azabicyclo[3.2.0]heptane-6-carboxylate and tert-butyl (cis)-4-hydroxy-6-azabicyclo[3.2.0]heptane-6-carboxylate. The alcohols may be separated using methods known to those of skill in the art of organic chemistry such as, but not limited to, chromatography and then oxidized under Swern conditions to provide the corresponding ketones. tert-Butyl (cis)-4-oxo-6-azabicyclo[3.2.0]heptane-6-carboxylate may be processed as described in Schemes 2-8 to provide azabicycles of general formula (220), (221), (222) and (223).

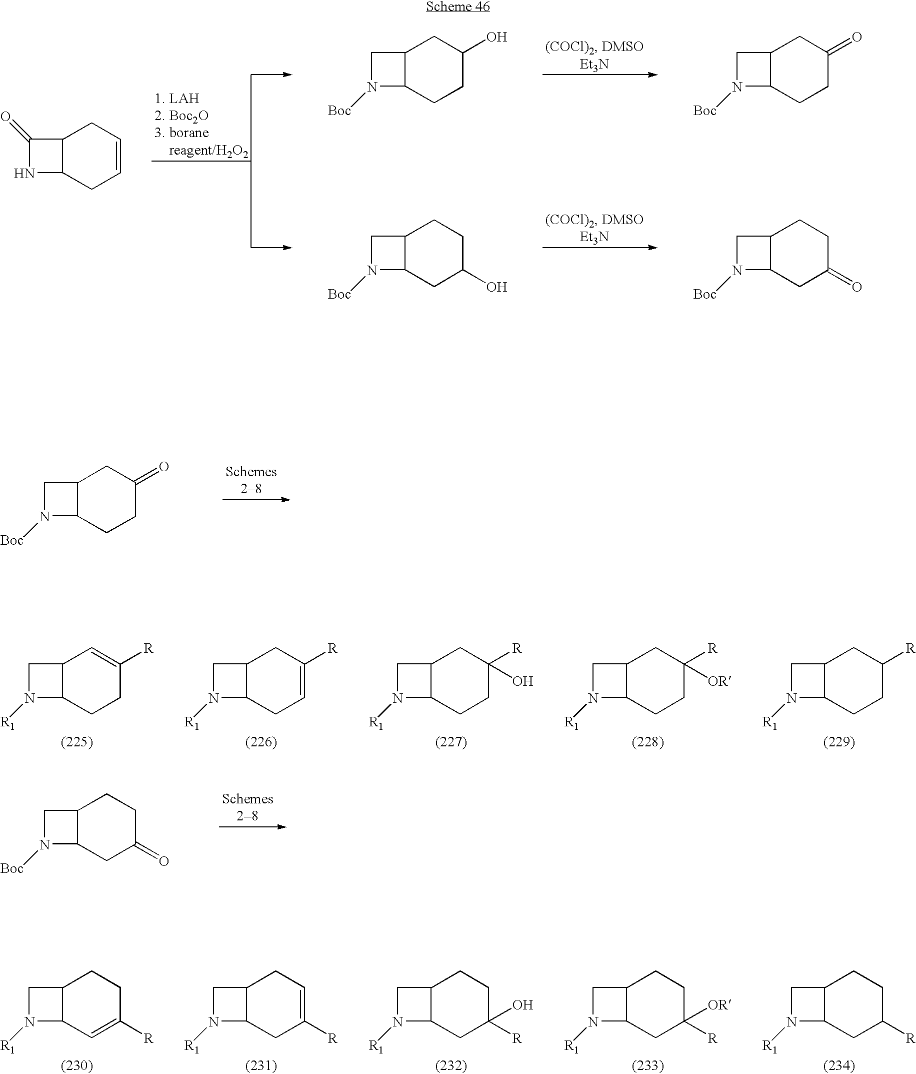

Azabicycles of general formula (225), (226), (227), (228), (229), (230), (231), (232), (233) and (234), wherein R is aryl or heterocycle, R′ is alkyl and R₁ is as defined in formula (I), may be prepared as described in Scheme 46. (cis)-7-Azabicyclo[4.2.0]oct-3-en-8-one, purchased from Maybridge or prepared as described in J. Am. Chem. Soc. (1968) 90(14), 3897-3898, may be treated with lithium aluminum hydride followed by di-tert-butyl dicarbonate and then treatment with a hydroborating agent such as, but not limited to, 9-borabicyclo[3.3.1]nonane to provide a stereoisomeric mixture of alcohols, tert-butyl (cis)-3-hydroxy-7-azabicyclo[4.2.0]octane-7-carboxylate and tert-butyl (cis)-4-hydroxy-7-azabicyclo[4.2.0]octane-7-carboxylate. The alcohols may be separated using methods known to those of skill in the art of organic chemistry such as, but not limited to, chromatography and then oxidized under Swern conditions to provide the corresponding ketones. tert-Butyl (cis)-3-oxo-7-azabicyclo[4.2.0]octane-7-carboxylate may be processed as described in Schemes 2-8 to provide azabicycles of general formula (225), (226), (227), (228) and (229). tert-Butyl (cis)-4-oxo-7-azabicyclo[4.2.0]octane-7-carboxylate may be processed as described in Schemes 2-8 to provide azabicycles of general formula (230), (231), (232), (233) and (234).

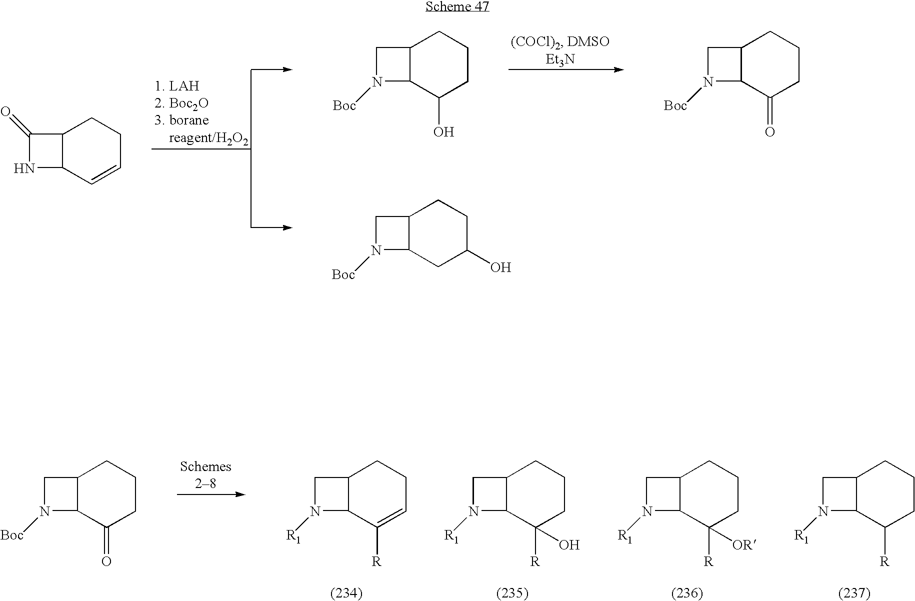

Azabicycles of general formula (234), (235), (236) and (237), wherein R is aryl or heterocycle, R′ is alkyl and R₁ is as defined in formula (I), may be prepared as described in Scheme 47. (cis)-7-Azabicyclo[4.2.0]oct-4-en-8-one, prepared as described in J. Chem. Soc., Perkin Trans. I (1977) 874-884, may be treated with lithium aluminum hydride followed by di-tert-butyl dicarbonate and then treatment with a hydroborating agent such as, but not limited to, 9-borabicyclo[3.3.1]nonane to provide a stereoisomeric mixture of alcohols, tert-butyl (cis)-4-hydroxy-7-azabicyclo[4.2.0]octane-7-carboxylate and tert-butyl (cis)-5-hydroxy-7-azabicyclo[4.2.0]octane-7-carboxylate. The alcohols may be separated using methods known to those of skill in the art of organic chemistry such as, but not limited to, chromatography and then oxidized under Swern conditions to provide the corresponding ketones. tert-Butyl (cis)-5-oxo-7-azabicyclo[4.2.0]octane-7-carboxylate may be processed as described in Schemes 2-8 to provide azabicycles of general formula (234), (235), (236) and (237).

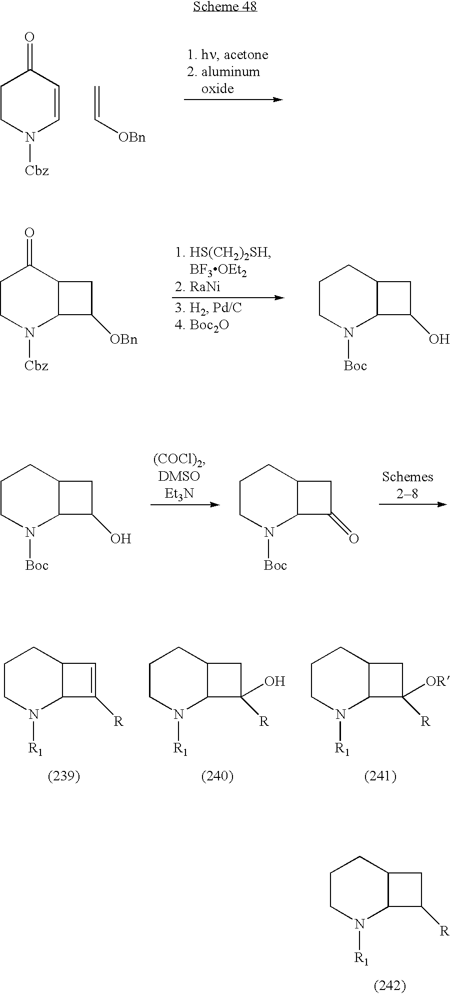

Azabicycles of general formula (239), (240), (241) and (242), wherein R is aryl or heterocycle, R′ is alkyl and R₁ is as defined in formula (I), may be prepared as described in Scheme 48. Benzyl 4-oxo-3,4-dihydro-[(2H)-pyridinecarboxylate, prepared as described in J. Chem. Soc., Perkin Trans. 1 (1977) 874-884, may be treated with benzyl vinyl ether in a photochemical [2+2] cycloaddition to provide benzyl (cis)-8-(benzyloxy)-5-oxo-2-azabicyclo[4.2.0]octane-2-carboxylate using methodology described in Helv. Chim. Acta (1991) 74, 163-177. Benzyl (cis)-8-(benzyloxy)-5-oxo-2-azabicyclo[4.2.0]octane-2-carboxylate, may be treated with 1,2-ethanediol and boron trifluoride diethyl etherate and then treated with Raney nickel to provide (cis)-2-azabicyclo[4.2.0]octan-8-ol. (cis)-2-Azabicyclo[4.2.0]octan-8-ol may be treated with a metal catalyst such as, but not limited to, palladium on carbon under a hydrogen atmosphere in the presence of di-tert-butyl dicarbonate and then oxidized under Swern conditions to provide tert-butyl (cis)-8-oxo-2-azabicyclo[4.2.0]octane-2-carboxylate. tert-Butyl (cis)-8-oxo-2-azabicyclo[4.2.0]octane-2-carboxylate may be processed as described in Schemes 2-8 to provide azabicycles of general formula (239), (240), (241) and (242).

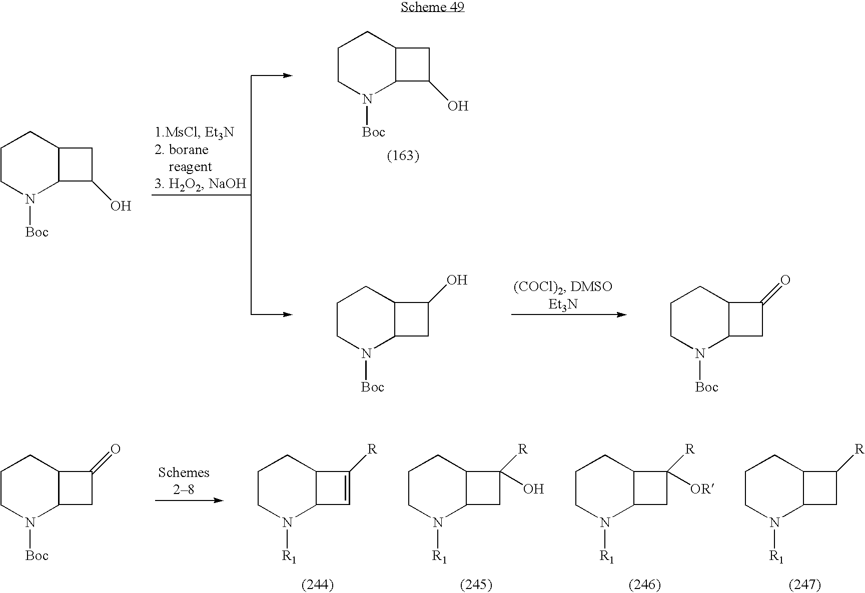

Azabicycles of general formula (244), (245), (246) and (247), wherein R is aryl or heterocycle, R′ is alkyl and R₁ is as defined in formula (I), may be prepared as described in Scheme 49. tert-Butyl (cis)-8-hydroxy-2-azabicyclo[4.2.0]octane-2-carboxylate, prepared as described in Scheme 48, may be treated with methanesulfonyl chloride and a base such as, but not limited to, triethylamine to provide tert-butyl (cis)-2-azabicyclo[4.2.0]oct-7-ene-2-carboxylate. tert-Butyl (cis)-2-azabicyclo[4.2.0]oct-7-ene-2-carboxylate may be treated with a borane reagent such as, but not limited to, 9-borabicyclo[3.3.1]nonane and then basic hydrogen peroxide to provide a mixture of stereoisomeric alcohols. The alcohols may be separated using methods known to those of skill in the art of organic chemistry such as, but not limited to, chromatography and then oxidized under Swern conditions to provide the corresponding ketones. tert-Butyl (cis)-7-oxo-2-azabicyclo[4.2.0]octane-2-carboxylate may be processed as described in Schemes 2-8 to provide azabicycles of general formula (244), (245), (246) and (247).

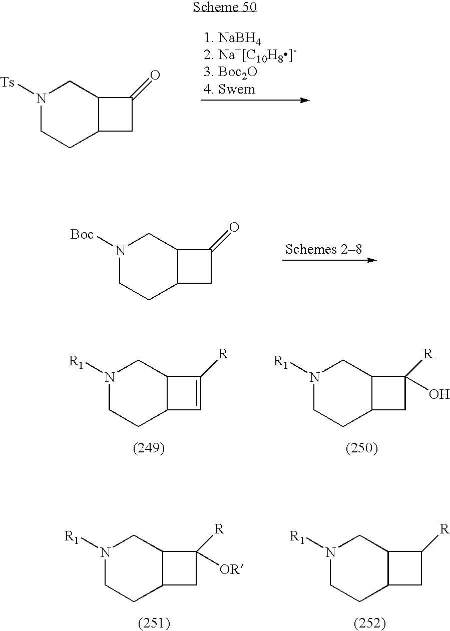

Azabicycles of general formula (249), (250), (251) and (252), wherein R is aryl or heterocycle, R′ is alkyl and R₁ is as defined in formula (I), may be prepared as described in Scheme 50. 3-[(4-Methylphenyl)sulfonyl]-3-azabicyclo[4.2.0]octan-8-one, prepared as described in Heterocycles (1989) 28(1), 29-32, may be treated with a reducing agent such as, but not limited to, sodium borohydride followed by sodium naphthalenide to remove the N-tosyl protecting group. The amine may be treated with di-tert-dicarbonate and then oxidized under Swern conditions (DMSO/oxalyl chloride/triethylamine) to provide to provide tert-butyl (cis)-8-oxo-3-azabicyclo[4.2.0]octane-3-carboxylate. tert-Butyl (cis)-8-oxo-3-azabicyclo[4.2.0]octane-3-carboxylate may be processed as described in Schemes 2-8 to provide azabicycles of general formula (249), (250), (251) and (252).

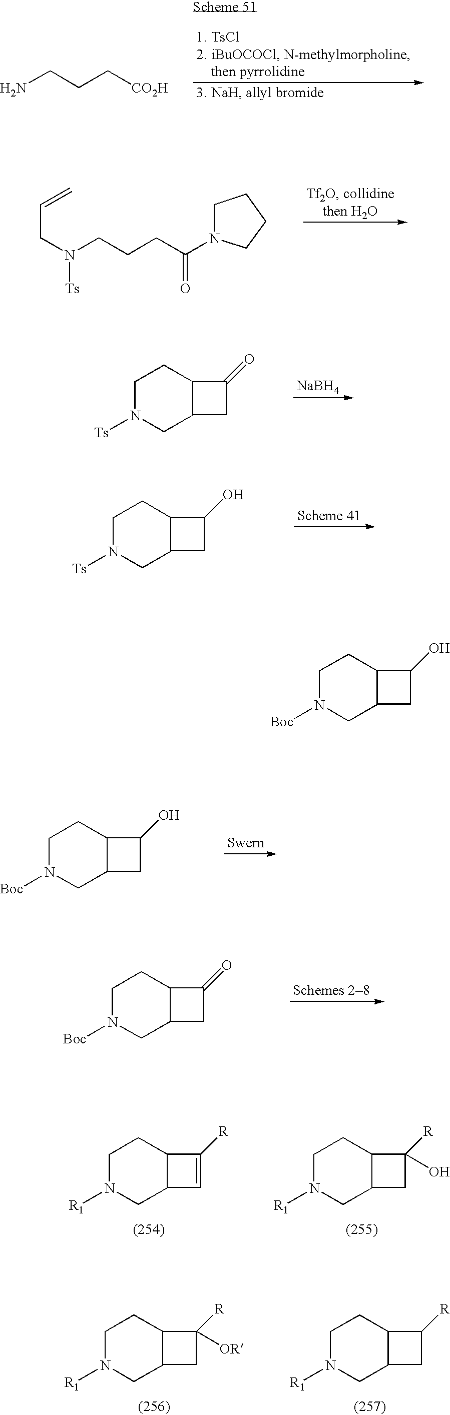

Azabicycles of general formula (254), (255), (256) and (257), wherein R is aryl or heterocycle, R′ is alkyl and R₁ is as defined in formula (I), may be prepared as described in Scheme 51. 3-[(4-Methylphenyl)sulfonyl]-3-azabicyclo[4.2.0]octan-7-one may be prepared from commercially available 4-aminobutyric acid utilizing synthetic methodology described in Heterocycles (1989) 28(1), 29-32. 4-Aminobutyric acid may be treated with 4-methyltoluenesulfonyl chloride to provide the N-protected butyric acid. The N-protected butyric acid may be treated with pyrrolidine and a chloroformate such as, but not limited to, isobutylchloroformate to provide the amide. The amide may be treate with a strong base such as, but not limited to, sodium hydride and allyl bromide to provide N-allyl-4-methyl-N-[4-oxo-4-(1-pyrrolidinyl)butyl]benzenesulfonamide. N-Allyl-4-methyl-N-[4-oxo-4-(1-pyrrolidinyl)butyl]benzenesulfonamide may be treated with trifluoromethanesulfonyl anhydride and a base such as, but not limited to, collidine to provide the iminium 2+2 product which may be treated with water to provide (cis)-3-[(4-methylphenyl)sulfonyl]-3-azabicyclo[4.2.0]octan-7-one. (cis)-3-[(4-Methylphenyl)sulfonyl]-3-azabicyclo[4.2.0]octan-7-one may be treated with a reducing agent such as, but not limited to, sodium borohydride to provide stereoisomeric alcohols. The stereoisomeric alcohols may be processed as described in Scheme 41 to provide the N-boc protected stereoisomeric alcohols. The N-boc protected stereoisomeric alcohols may be oxidized under Swern conditions (DMSO/oxalyl chloride/triethylamine) to provide tert-butyl (cis)-7-oxo-3-azabicyclo[4.2.0]octane-3-carboxylate. tert-Butyl (cis)-7-oxo-3-azabicyclo[4.2.0]octane-3-carboxylate may be processed as described in Scheme 2-8 to provide azabicycles of general formula (254), (255), (256) and (257).

The compounds and processes of the present invention will be better understood by reference to the following Examples, which are intended as an illustration of and not a limitation upon the scope of the invention.

### EXAMPLE 1

### (cis)-5-(6-chloro-3-pyridinyl)-1,2,3,3a,4,6a-hexahydrocyclopenta[c]pyrrole Hydrochloride

### Example 1A

### (cis)-2,3,3a,4,7,7a-hexahydro-1H-isoindole

A suspension of lithium aluminum hydride (21.4 g, 0.562 mol) in THF (700 mL) at room temperature was treated with cis-1,2,3,6-tetrahydrophthalimide (Aldrich; 37 g, 0.245 mol) in small portions. The reaction mixture was stirred at 60° C. overnight then cooled to room temperature and quenched carefully by the sequential addition of water (22 mL), THF (22 mL), 15% aqueous KOH (22 mL), and water (80 mL). The mixture was diluted with diethyl ether (100 mL), stirred at room temperature for 1 hour, and then filtered (methylene chloride wash). The filtrate was concentrated under reduced pressure to provide the title compound as an oil (26.1 g). ¹H NMR (CDCl₃, 300 MHz) δ 1.76-2.02 (m, 2H), 2.10-2.33 (m, 3H), 2.34-2.56 (m, 2H), 2.61-2.79 (m, 2H), 2.87-3.17 (m, 2H), 5.53-6.03 (m, 2H); MS (DCI/NH₃) m/Z 124 (M+H)⁺.

### Example 1B

### tert-butyl (cis)-1,3,3a,4,7,7a-hexahydro-2H-isoindole-2-carboxylate

The product from Example 1A (26 g, 0.21 mol) in methylene chloride (250 mL) at 0° C. was treated with di-tert-butyl dicarbonate (46.1 g, 0.21 mol). The solution was stirred for 30 minutes at 0° C. and then 2 hours at ambient temperature. The reaction mixture was partitioned between methylene chloride and saturated aqueous sodium bicarbonate. The organic extract was washed with saturated aqueous sodium bicarbonate and brine, dried over Na₂SO₄, filtered and the filtrate was concentrated under reduced pressure. The residue was purified by chromatography (SiO₂, 15% ethyl acetate/hexane) to provide the title compound (36 g). ¹H NMR (CDCl₃, 300 MHz) δ 1.45 (s, 9H), 1.84-1.99 (m, 2H), 2.11-2.40 (m, 4H), 3.07 (m, 1H), 3.18 (m, 1H), 3.33-3.53 (m, 2H), 5.60 (bs, 2H); MS (DCI/NH₃) m/z 224 (M+H)⁺.

### Example 1C

### tert-butyl (cis)-3,4-bis(carboxymethyl)-1-pyrrolidinecarboxylate

The product from Example 1B (36 g, 0.16 mol) in carbon tetrachloride (360 mL), acetonitrile (360 mL), and water (540 mL) was treated with sodium periodate (138 g, 0.645 mol) followed by catalytic ruthenium (IV) oxide hydrate (0.885 g, 0.0066 mol). The mixture was stirred vigorously for 24 hours at ambient temperature then diluted with methylene chloride (500 mL) and water (100 mL), and filtered through diatomaceous earth. The filtrate was pass through a small plug of SiO₂ (methylene chloride wash), then evaporated under reduced pressure. The solid was crystallized from diethyl ether to provide the title compound (23.3 g). ¹H NMR (CD₃OH, 300 MHz) δ 1.45 (s, 9H), 2.18-2.32 (m, 2H), 2.37-2.52 (m, 2H), 2.52-2.76 (m, 2H), 3.05-3.21 (m, 2H), 3.35-3.56 (m, 2H), 3.79-3.89 (m, 1H), 6.11 (m, 1H); MS (DCI/NV ₃) m/z 288 (M+H)⁺.

### Example 1D

### Tert-butyl (cis)-5-oxohexahydrocyclopenta[c]pyrrole-2(1H)-carboxylate

The product from Example 1C (23 g) in acetic anhydride (140 mL) was treated with sodium acetate (5.3 g, 0.065 mol). The reaction mixture was stirred at 120° C. for 3 hours then cooled to ambient temperature, filtered (diethyl ether wash) and the filtrate was evaporated under reduced pressure. The residue was purified by chromatography (SiO₂, 30% ethyl acetate/hexane) to provide the title compound (11.7 g). ¹H NMR (CDCl₃, 300 MHz) δ 1.45 (s, 9H), 2.12 (m, 1H), 2.19 (m, 1H), 2.44 (m, 1H), 2.51 (m, 1H), 2.93 (m, 2H), 3.22 (m, 2H), 3.65 (m, 2H); MS (DCI/NH₃) m/z 226 (M+H)⁺.

### Example 1E

### Tert-butyl 5-hydroxy-5-(3-pyridinyl)hexahydrocyclopenta[c]pyrrole-2(1H)-carboxylate

2-Chloro-5-iodopyridine (1.38 g, 5.75 mmol), prepared as described in Tetrahedron Letters, 34, 7493-7496 (1993), in THF (7 mL) was treated with n-butyl lithium (2.5M solution in hexanes, 2.30 mL, 5.75 mmol) dropwise over 20 minutes at −78° C. After stirring at −78° C. for 40 minutes, the mixture was treated dropwise with the product from Example 1D (0.259 g, 1.15 mmol) in diethyl ether (3 mL) dropwise. The reaction mixture was stirred at −78° C. for 30 minutes and then allowed to warm to 0° C. The mixture was quenched with saturated aqueous ammonium chloride and allowed to warm to room temperature. The mixture was partitioned between ethyl acetate and saturated aqueous ammonium chloride. The organic extract was separated, washed with saturated ammonium chloride, water, brine, dried over Na₂SO₄, filtered and the filtrate was concentrated under reduced pressure. The residue was purified by chromatography (SiO₂, 50% ethyl acetate/hexane) to provide the title compound (0.255 g). MS (DCI/NH₃) m/z 339 (M+H)⁺.

### Example 1F

### Tert-butyl (cis)-5-(6-chloro-3-pyridinyl)-3,3a,4,6a-tetrahydrocyclopenta[c]pyrrole-2(1H)-carboxylate

The product from Example 1E (0.098 g, 0.290 mmol) in methylene chloride (4 mL) was treated with methanesulfonyl chloride (0.045 mL, 0.58 mmol), triethylamine (0.080 mL, 0.609 mmol) and catalytic DMAP (0.001 g). After stirring at ambient temperature for 16 hours, the volatiles were evaporated under reduced pressure. The residue was purified by chromatography (SiO₂, 50% ethyl acetate/hexane) to provide the title compound (0.036 g, 39%). MS (DCI/NH₃) m/z 321 (M+H)⁺.

### Example 1G

### (cis)-5-(6-chloro-3-pyridinyl)-1,2,3,3a,4,6a-hexahydrocyclopenta[c]pyrrole Hydrochloride

The product from Example 1F (0.036 g, 0.112 mmol) in methanol (1 mL) was treated with a 1 M solution of HCl in diethyl ether (2 mL). After stirring at ambient temperature for 2 hours, the volatiles were evaporated under reduced pressure. The residue was crystallized from ethanol and diethyl ether to provide the title compound (0.034 g, 100%). ¹H NMR (CD₃OD, 300 MHz) δ 2.75 (m, 1H), 3.15 (m, 2H), 3.32-3.55 (m, 4H), 3.82 (m, 1H), 6.24 (m, 1H), 7.46 (d, J=8.46 Hz, 1H), 7.95 (dd, J=2.57, 8.46 Hz, 1H), 8.47 (d, J=2.21 Hz, 1H); MS (DCI/NH₃) m/z 221 (M+H)⁺; Anal. calculated for C₁₂H₁₃ClN₂.1.8HCl: C, 50.34; H, 5.21; N, 9.78. Found: C, 50.40; H, 5.35; N, 9.61.

### EXAMPLE 2

### (cis)-5-(5-methoxy-3-pyridinyl)-1,2,3,3a,4,6a-hexahydrocyclopenta[c]pyrrole Hydrochloride

### Example 2A

### tert-butyl (cis)-5-hydroxy-5-(5-methoxy-3-pyridinyl)hexahydrocyclopenta[c]pyrrole-2(1H)-carboxylate

n-Butyl lithium (2.5M solution in hexanes, 1.5 mL, 3.81 mmol) was added dropwise over 20 minutes to 3-bromo-5-methoxypyridine (0.717 g, 3.81 mmol), prepared as described in WO 00/44755 in THF (4 mL) at −78° C. After stirring at −78° C. for 40 minutes, the product from Example 1D (0.286 g, 1.27 mmol) in diethyl ether (4 mL) was added dropwise to the reaction mixture and stirred at −78° C. for an additional 30 minutes. The reaction mixture was then allowed to warm to 0° C. and then quenched with saturated aqueous ammonium chloride and allowed to warm to room temperature. The mixture was partitioned between ethyl acetate and saturated aqueous ammonium chloride. The organic layer was separated, washed with saturated ammonium chloride, water, brine, dried over Na₂SO₄, filtered and the filtrate was concentrated under reduced pressure. The residue was purified by chromatography (SiO₂, 50% ethyl acetate/hexane) to provide the title compound (0.142 g, 33%). MS (DCI/NH₃) m/z 335 (M+H)⁺.

### Example 2B

### Tert-butyl (cis)-5-(5-methoxy-3-pyridinyl)-3,3a,4,6a-tetrahydrocyclopenta[c]pyrrole-2(1H)-carboxylate

The product from Example 2A (0.207 g, 0.619 mmol) in methylene chloride (6 mL) was treated with methanesulfonyl chloride (0.144 mL, 1.86 mmol), triethylamine (0.27 mL, 1.92 mmol) and catalytic DMAP (0.002 g). After stirring at ambient temperature for 16 hours, the volatiles were evaporated under reduced pressure. The residue was purified by chromatography (SiO₂, 50% ethyl acetate/hexane) to provide the title compound (0.038 g, 19%). MS (DCI/NH₃) m/z 317 (M+H)⁺.

### Example 2C

### (cis)-5-(5-methoxy-3-pyridinyl)-1,2,3,3a,4,6a-hexahydrocyclopenta[c]pyrrole Hydrochloride

The product from Example 2B (0.038 g, 0.120 mmol) in methanol (1 mL) was treated with 1M solution of HCl in diethyl ether (2 mL) and stirred at ambient temperature for 2 hours. The volatiles were evaporated under reduced pressure. The residue was crystallized from ethanol and diethyl ether to provide the title compound (0.022 g, 64%). ¹H NMR (CD₃OD, 300 MHz) δ 2.82 (m, 1H), 3.17-3.27 (m, 2H), 3.33-3.61 (m, 4H), 3.88 (m, 1H), 4.07 (s, 3H), 6.54 (m, 1H), 8.17 (bs, 1H), 8.49 (bs, 1H), 8.57 (bs, 1H); MS (DCI/NH₃) m/z 217 (M+H)⁺; Anal. calculated for C₁₃H₁₆ON₂.2.2HCl: C, 52.66; H, 6.19; N, 9.45. Found: C, 52.45; H, 5.99; N, 9.17.

### EXAMPLE 3

### (cis)-5-(6-chloro-5-methyl-3-pyridinyl)-1,2,3,3a,4,6a-hexahydrocyclopenta[c]pyrrole Hydrochloride

### Example 3A

### Tert-butyl 5-(6-chloro-5-methyl-3-pyridinyl)-5-hydroxyhexahydrocyclopenta[c]pyrrole-2(1H)-carboxylate

n-Butyl lithium (2.5 M solution in hexanes, 1.31 mL, 3.27 mmol) was added dropwise over 20 minutes to a −78° C. solution of 5-bromo-2-chloro-3-methylpyridine (0.675 g, 3.27 mmol) in THF (4 mL). After stirring at −78° C. for 40 minutes, the product from Example 1D (0.368 g, 1.64 mmol) in THF (3 mL) was added dropwise to the reaction mixture. After stirring at −78° C. for 30 minutes, the reaction mixture was allowed to warm to 0° C. and then was quenched with saturated aqueous ammonium chloride and allowed to warm to room temperature. The mixture was partitioned between ethyl acetate and saturated aqueous ammonium chloride. The organic layer was separated, washed with saturated ammonium chloride, water, brine, dried over Na₂SO₄, filtered and the filtrate was concentrated under reduced pressure. The residue was purified by chromatography (SiO₂, 50% ethyl acetate/hexane) to provide the title compound (0.300 g, 52%). MS (DCI/NH₃) m/z 353 (M+H)⁺.

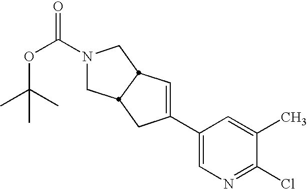

### Example 3B

### Tert-butyl (cis)-5-(6-chloro-5-methyl-3-pyridinyl)-3,3a,4,6a-tetrahydrocyclopenta[c]pyrrole-2(1H)-carboxylate

The product from Example 3A (0.300 g, 0.852 mmol) in methylene chloride (5 mL) was treated with methanesulfonyl chloride (0.198 mL, 2.56 mmol), triethylamine (0.38 mL, 2.73 mmol) and catalytic DMAP (0.003 g). The reaction mixture was stirred at ambient temperature for 16 hours and then the volatiles were evaporated under reduced pressure. The residue was purified by chromatography (SiO₂, 50% ethyl acetate/hexane) to provide the title compound (0.167 g, 59%). MS (DCI/NH₃) m/z 335 (M+H)⁺.

### Example 3C

### (cis)-5-(6-chloro-5-methyl-3-pyridinyl)-1,2,3,3a,4,6a-hexahydrocyclopenta[c]pyrrole Hydrochloride

The product from Example 3B (0.167 g, 0.500 mmol) in methanol (1 mL) was treated with 1 M solution of HCl in diethyl ether (3 m-L). After stirring at ambient temperature for 2 hours, the volatiles were evaporated under reduced pressure. The residue was crystallized from ethanol and diethyl ether to provide the title compound (0.095 g, 70%). ¹H NMR (CD₃OD, 300 MHz) δ 2.40 (s, 3H), 2.74 (m, 1H), 3.11-3.20 (m, 2H), 3.36-3.54 (m, 4H), 3.81 (m, 1H), 6.2 (m, 1H), 7.87 (d, J=2.4 Hz, 1H), 8.30 (d, J=2.6 Hz, 1H); MS (DCI/NH₃) m/z 235 (M+H)⁺; Anal. calculated for C₁₃H₁₅ClN₂.1.1HCl: C, 56.81; H, 5.90; N, 10.19. Found: C, 56.52; H, 5.90; N, 9.97.

### EXAMPLE 4

### (cis)-5-(3-pyridinyl)-1,2,3,3a,4,6a-hexahydrocyclopenta[c]pyrrole Hydrochloride

### Example 4A

### Tert-butyl (cis)-5-{[(trifluoromethyl)sulfonyl]oxy}-3,3a,4,6a-tetrahydrocyclopenta[c]pyrrole-2(1H)-carboxylate

The product from Example 1D (3.0 g, 13.3 mmol) in THF (80 mL) was treated with lithium bis(trimethylsilyl)amide (1.0M solution in THF, 17.3 mL, 17.3 mmol) at −78° C. The reaction mixture was stirred at −78° C. for 30 minutes and then a solution of N-phenyltrifluoromethanesulfonimide (6.65 g, 18.6 mmol) in THF (20 mL) was added slowly. The reaction mixture was stirred at −78° C. for 1 hour and then warmed to −10° C. and stirred for an additional hour. The volatiles were removed under reduced pressure, and the residue was purified by chromatography (SiO₂, 10% ethyl acetate/hexane) to provide the title compound as a white solid (4.44 g, 94%). MS (DCI/NH₃) m/z 358 (M+H)⁺.

### Example 4B

### Tert-butyl (cis)-5-(trimethylstannyl)-3,3a,4,6a-tetrahydrocyclopenta[c]pyrrole-2(1H)-carboxylate

The product from Example 4A (1.00 g, 2.80 mmol) in THF (15 mL) was treated with hexamethylditin (0.826 g, 2.52 mmol), lithium chloride (0.712 g, 16.8 mmol) and tetrakis(triphenylphosphine)palladium(0) (0.065 g, 2 mol %) and stirred at 60° C. for 16 hours. The reaction mixture was allowed to cool to ambient temperature, diluted with diethyl ether, washed with 1N aqueous sodium hydroxide, water, brine, dried over Na₂SO₄, filtered and the filtrate was concentrated under reduced pressure. The residue was purified by chromatography (SiO₂, 10% ethyl acetate/hexane) to provide the title compound as a white solid (0.769 g, 74%). MS (DCI/NH₃) m/z 372, 374 (M+H)⁺.

### Example 4C

### Tert-butyl (cis)-5-(3-pyridinyl)-3,3a,4,6a-tetrahydrocyclopenta[c]pyrrole-2(1H)-carboxylate

3-Bromopyridine (0.129 g, 0.82 mmol), triphenylarsine (0.020 g, 0.065 mmol) and tris(dibenzylideneacetone)dipalladium(0) (0.015 g, 2 mol %) in anhydrous 1-methyl-2-pyrrolidinone (4 mL) were treated with the product from Example 4B (0.305 g, 0.82 mmol) in 1-methyl-2-pyrrolidinone (2 mL). The reaction mixture was stirred at 60° C. for 16 hours, allowed to cool to ambient temperature and partitioned between ethyl acetate and 1N aqueous sodium hydroxide. The organic portion was separated, washed with 1N aqueous sodium hydroxide, water, brine, dried over Na₂SO₄, filtered and the filtrate was evaporated under reduced pressure. The residue was purified by chromatography (SiO₂, 50% ethyl acetate/hexane) to provide the title compound (0.163 g, 69%). MS (DCI/NH₃) m/z 287 (M+H)⁺.

### Example 4D

### (cis)-5-(3-pyridinyl)-1,2,3,3a,4,6a-hexahydrocyclopenta[c]pyrrole hydrochloride hydrochloride

The product from Example 4C (0.163 g, 0.570 mmol) in methanol (1 mL) was treated with a 1M solution of HCl in diethyl ether (2 mL). After stirring at ambient temperature for 2 hours, the volatiles were evaporated under reduced pressure. The residue was crystallized from ethanol and diethyl ether to provide the title compound (0.138 g, 93%). ¹H NMR (CD₃OD, 300 MHz) δ 2.85 (m, 1H), 3.19-3.25 (m, 2H), 3.35-3.59 (m, 4H), 3.89 (m, 1H), 6.56 (m, 1H), 8.09 (dd, J=5.8, 8.5 Hz, 1H), 8.75 (m, 1H), 8.97 (d, J=1.4 Hz, 1H); MS (DCI/NH₃) m/z 187 (M+H)⁺; Anal. calculated for C₁₂H₁₄N₂.2HCl-0.2H₂O: C, 54.85; H, 6.29; N, 10.66. Found: C, 54.73; H, 6.66; N, 10.58.

### EXAMPLE 5

### (cis)-5-(3-methyl-5-isoxazolyl)-1,2,3,3a,4,6a-hexahydrocyclopenta[c]pyrrole Hydrochloride

### Example 5A

### Tert-butyl 5-(3-methyl-5-isoxazolyl)-3,3a,4,6a-tetrahydrocyclopenta[c]pyrrole-2(1H)-carboxylate

The product from Example 4A (411 mg, 1.15 mmol), triphenylarsine (28 mg, 0.092 mmol) and tris(dibenzylideneacetone)dipalladium(0) (21 mg, 0.023 mmol) in anhydrous 1-methyl-2-pyrrolidinone (6 mL) were treated with 3-methyl-5-(tributylstannyl)isoxazole (448 mg, 1.2 mmol, prepared according to Sakamoto, T.; et. al., Tetrahedon, 1991, 47, 5111) in 1-methyl-2-pyrrolidinone (2 mL). The reaction mixture was stirred at 60° C. overnight, allowed to cool to ambient temperature and 1M aqueous KF (1.5 mL) was added. The mixture was stirred for 0.5 hours, diluted with ethyl acetate and filtered through Celite. The filtrate was washed with water (5×), brine, dried over Na₂SO₄, filtered and the filtrate was concentrated under reduced pressure. The residue was purified by chromatography (silica gel, gradient from hexane to 30% EtOAc/hexane) to provide 220 mg (66%) of the title compound. MS (DCI/NH₃) m/z 291 (M+H)⁺.

### Example 5B

### (cis)-5-(3-methyl-5-isoxazolyl)-1,2,3,3a,4,6a-hexahydrocyclopenta[c]pyrrole hydrochloride

The product from Example 5A (220 mg, 0.759 mmol) in ethyl acetate (5 mL) was treated with a 4M solution of HCl in 1,4-dioxane (1 mL). After stirring at ambient temperature for 1 hour, a few drops of diethyl ether were added resulting in precipitation of a white solid that was collected by filtration and dried at 60° C. to provide 126 mg (73%) of the hydrochloride salt. ¹H NMR (CD₃OD, 300 MHz) δ 2.27 (s, 3H), 2.70 (m, 1H), 3.07-3.20 (m, 2H), 3.35-3.55 (m, 4H), 3.83 (m, 1H), 6.26 (d, J=2.0 Hz, 1H), 6.31 (s, 1H); MS (DCI/NH₃) m/Z 191 (M+H)⁺; Anal. calculated for C₁₁H₁₄N₂O.HCl: C, 58.28; H, 6.67; N, 12.36. Found: C, 58.18; H, 6.55; N, 12.27.

### EXAMPLE 6

### (cis)-5-(5,6-dichloro-3-pyridinyl)-1,2,3,3a,4,6a-hexahydrocyclopenta[c]pyrrole hydrochloride

### Example 6A

### Tert-butyl 5-(5,6-dichloro-3-pyridinyl-3,3a,4,6a-tetrahydrocyclopenta[c]pyrrole-2(1H)-carboxylate

2,3-Dichloro-5-iodopyridine (0.288 g, 1.05 mmol), triphenylarsine (0.026 g, 0.084 mmol), and tris(dibenzylideneacetone)dipalladium(0) (0.019 g, 2 mol %) in anhydrous 1-methyl-2-pyrrolidinone (5 mL) were treated with the product from Example 4B (0.392 g, 1.05 mmol) in 1-methyl-2-pyrrolidinone (2 mL). After stirring at 60° C. for 5 hours, the reaction mixture was allowed to cool to ambient temperature and partitioned between ethyl acetate and 1N aqueous sodium hydroxide. The organic extract was separated, washed with 1N aqueous sodium hydroxide, water, brine, dried over Na₂SO₄, filtered and the filtrate was concentrated under reduced pressure. The residue was purified by chromatography (SiO₂, 50% ethyl acetate/hexanes) to provide the title compound (0.228 g, 61%). MS (DCI/NH₃) m/z 355 (M+H)⁺.

### Example 6B

### (cis)-5-(5,6-dichloro-3-pyridinyl)-1,2,3,3a,4,6a-hexahydrocyclopenta[c]pyrrole Hydrochloride

The product from Example 6A (0.228 g, 0.642 mmol) in methanol (1 mL) was treated with a 1 M solution of HCl in diethyl ether (2 mL). After stirring at ambient temperature for 2 hours, the volatiles were evaporated under reduced pressure. The residue was crystallized from ethanol and diethyl ether to provide the title compound (0.120 g, 73%). ¹H NMR (CD₃OD, 300 MHz) δ 2.75 (m, 1H), 3.17 (m, 2H), 3.31-3.55 (m, 4H), 3.83 (m, 1H), 6.30 (m, 1H), 8.10 (d, J=2.0 Hz, 1H), 8.44 (d, J=2.4 Hz, 1H); MS (DCI/NH₃) m/z 255 (M+H)⁺; Anal. calculated for C₁₂H₁₂Cl₂N₂.HCl: C, 49.43; H, 4.49; N, 9.61. Found: C, 49.32; H, 4.60; N, 9.38.

### EXAMPLE 7

### (cis)-2-methyl-5-(3-methyl-5-isoxazolyl)-1,2,3,3a,4,6a-hexahydrocyclopenta[c]pyrrole Hydrochloride

The product from Example 5B (22 mg, 0.11 mmol), 37% formaldehyde in water and formic acid were stirred at reflux for 2 hours, allowed to cool to room tempreature and concentrated under reduced pressure. The residue was purified by chromatography (silica gel, 98:2:0.1 CH₂Cl₂/MeOH/NH₄OH) to provide 13 mg (54%) of the free base that was processed as described in Example 5B to provide the title compound as the hydrochloride salt. ¹H NMR (CD₃OD, 300 MHz) δ 2.28 (s, 3H), 2.70 (m, 1H), 2.90 (s, 3H), 3.07-3.22 (m, 2H), 3.30-3.60 (m, 4H), 3.84 (m, 1H), 6.25 (br s, 1H), 6.32 (s, 1H); MS (DCI/NH₃) m/z 205 (M+H)⁺; Anal. calculated for C₁₁H₁₄N₂O.HCl.0.25H₂O: C, 58.77; H, 7.19; N, 11.42. Found: C, 59.09; H, 6.91; N, 11.49.

### EXAMPLE 8

### (cis)-5-(5-chloro-3-pyridinyl)-1,2,3,3a,4,6a-hexahydrocyclopenta[c]pyrrole Dihydrochloride

### Example 8A

### (cis)-5-(5-chloro-3-pyridinyl)-1,2,3,3a,4,6a-hexahydrocyclopenta[c]pyrrole

The product from Example 4B and 5-chloro-3-pyridinyl trifluoromethanesulfonate, prepared as described in (WO 9740012), were processed as described in Example 4C to provide the title compound (42% yield) as a light yellow oil: MS (DCI/NH₃) m/z 321, 323 (M+H)⁺.

### Example 8B

### (cis)-5-(5-chloro-3-pyridinyl)-1,2,3,3a,4,6a-hexahydrocyclopenta[c]pyrrole Dihydrochloride

The product from Example 8A (190 mg, 0.59 mmol) in 10 mL of 1:1 EtOAc/EtOH was treated with a 4M solution of HCl in 1,4-dioxane (1.5 mL, 3.8 mmol). The mixture was refluxed for 1 hour and then allowed to cool to room temperature and stirred overnight. The white solid was collected by filtration to provide 170 mg of the title compound as the dihydrochloride salt. ¹H NMR (CD₃OD, 400 MHz) δ 2.84 (m, 1H), 3.15-3.24 (m, 2H), 3.30-3.60 (m, 4H), 3.88 (m, 1H), 6.54 (m, 1H), 8.62 (d, J=1.8 Hz, 1H), 8.86-8.88 (m, 2H); MS (DCI/NH₃) m/z 221,223 (M+H)⁺; Anal. calculated for C₁₂H₁₃ClN₂.2HCl: C, 49.09; H, 5.15; N, 9.54. Found: C, 48.83; H, 4.89; N, 9.35.

### EXAMPLE 9

### (cis)-5-(3-bromo-1,2,4-thiadiazol-5-yl)-1,2,3,3a,4,6a-hexahydrocyclopenta[c]pyrrole Hydrochloride

3-Bromo-5-chloro-1,2,4-thiadiazole (Acros; 19.7 mg, 0.099 mmol) in 1-methyl-2-pyrrolidinone (0.2 mL) was treated with 0.1 mL of a solution of tris(dibenzylideneacetone)dipalladium(0) (8.6 mg, 0.02 mmol), triphenylarsine (11.5 mg, 0.08 mmol) and copper(I) iodide (1.8 mg, 0.02 mmol) in 1-methyl-2-pyrrolidinone (0.5 mL). The reaction mixture was then treated with the product from Example 4B (35 mg, 0.094 mmol) in 1-methyl-2-pyrrolidinone (0.1 mL). The reaction mixture was warmed to 60° C., stirred over night and then allowed to cool to room temperature. The mixture was diluted with CH₂Cl₂ and washed with 0.5 mL of 1.0N aqueous NaOH. The aqueous phase was extracted twice with 5 ML of CH₂Cl₂. All the organic phases were combined and filtered through a 900 mg silica gel cartridge (Alltech). The solvent was removed under reduced pressure and the residue was purified by reverse phase preparative HPLC (Waters Nova-Pak HR C186 m 60 25×100 mm, 50-95% MeCN/10 mM NH₄OAc over 10 minutes at 40 mL/min) to provide the free base. The free base was dissolved in MeOH (0.3 mL) and treated with a 1.0M solution of HCl in diethyl ether (0.5 mL, 0.5 mmol). The solution was agitated at 40° C. for 16 hours resulting in the formation of a precipitate. The precipitate was triturated with diethyl ether (2×) and dried under reduced pressure to provide 20.9 mg (82%) of the title compound. ¹H NMR (CD₃OD) δ 2.82 (ddd, J=2.3, 4.2, 16.9 Hz, 1H), 3.18 (dd, J=5.2, 12.0 Hz, 1H), 3.21-3.26 (m, 1H), 3.34 (m, 1H), 3.40 (dd, J=3.1, 12.2 Hz, 1H), 3.47 (dd, J=8.3, 12.0 Hz, 1H), 3.53 (dd, J=8.7, 12.1, 1H), 3.89 (tt, J=2.7, 8.1 Hz, 1H), 6.69 (dd, J=2.3, 4.2 Hz, 1H); MS (ESI) m/z 272, 274 (M+H)⁺.

### EXAMPLE 10

### (cis)-5-(1,3-thiazol-2-yl)-1,2,3,3a,4,6a-hexahydrocyclopenta[c]pyrrole Hydrochloride

The product from Example 4B and 2-bromothiazole (purchased from Aldrich) were processed as described in Example 9 to provide the title compound. ¹H NMR (CD₃OD) δ 2.86 (ddd, J=2.2, 4.2, 16.7 Hz, 1H), 3.16 (dd, J=5.0, 11.6 Hz, 1H), 3.21-3.26 (m, 1H), 3.37 (dd, J=3.1, 11.6 Hz, 1H), 3.43 (dd, J=8.1, 11.6 Hz, 1H), 3.51 (dd, J=8.7, 11.6 Hz, 1H), 3.84 (tt, J=2.6, 8.0 Hz, 1H), 6.37 (dd, J=2.2, 4.1 Hz, 1H), 7.56 (d, J=3.4 Hz, 1H), 7.80 (d, J=3.4 Hz, 1H); MS (ESI) m/z 193 (M+H)⁺.

### EXAMPLE 11

### (cis)-5-(3,5-dimethyl-4-isoxazolyl)-1,2,3,3a,4,6a-hexahydrocyclopenta[c]pyrrole Hydrochloride

The product from Example 4B and 3,5-dimethyl-4-iodoisoxazole (purchased from Avocado) were processed as described in Example 9 to provide the title compound. ¹H NMR (CD₃OD) δ 2.30 (s, 3H), 2.46 (s, 3H), 2.67 (ddd, J=1.8, 4.2, 16.5 Hz, 1H), 3.08-3.14 (m, 2H), 3.21 (m, 1H), 3.38 (dd, J=7.8, 11.6 Hz, 1H), 3.47 (dd, J=8.7, 11.6 Hz, 1H), 3.73 (m, 1H), 5.73 (dd, J=2.0, 3.9 Hz, 1H); MS (ESI) m/z 205 (M+H)⁺.

### EXAMPLE 12

### (cis)-5-(1H-imidazol-4-yl)-1,2,3,3a,4,6a-hexahydrocyclopenta[c]pyrrole Hydrochloride

The product from Example 4B and 4-iodoimidazole (purchased from Combi-Blocks) were processed as described in Example 9 to provide the title compound. ¹H NMR (CD₃OD) δ 2.71 (dd, J=1.9, 16.5 Hz, 1H), 3.10 (tt, J=2.1, 8.4 Hz, 1H), 3.15 (dd, J=5.5, 12.0 Hz, 1H), 3.35 (dd, J=2.8, 11.5 Hz, 1H), 3.46 (dd, J=8.1, 11.6 Hz, 1H), 3.53 (dd, J=8.7, 11.6 Hz, 1H), 3.84 (m, 1H), 6.19 (dd, J=2.0, 3.9 Hz, 1H), 7.58 (s, 1H), 8.91 (s, 1H); MS (ESI) m/z 176 (M+H)⁺.

### EXAMPLE 13

### (cis)-5-(1,3-thiazol-5-yl)-1,2,3,3a,4,6a-hexahydrocyclopenta[c]pyrrole Hydrochloride

The product from Example 4B and 5-bromothiazole, prepared as described in (Recl. Trav. Chim. Pays-Bas, (1954), 73, 325), were processed as described in Example 9 to provide the title compound. ¹H NMR (CD₃OD) δ 2.79 (d, J=16.5 Hz, 1H), 3.16-3.22 (m, 2H), 3.32-3.38 (m, 2H), 3.43 (dd, J=8.0, 11.7 Hz, 1H), 3.52 (dd, J=8.7, 11.5 Hz, 1H), 3.83 (m, 1H), 6.18 (s, 1H), 8.14 (s, 1H), 9.51 (s, 1H); MS (ESI) m/z 193 (M+H)⁺.

### EXAMPLE 14

### (cis)-5-(imidazo[1,2-a]pyridin-3-yl)-1,2,3,3a,4,6a-hexahydrocyclopenta[c]pyrrole Hydrochloride

The product from Example 4B and 3-bromoimidazo[1,2-a]pyridine, prepared as described in J. Org. Chem. (1965), 30, 4085-4090, were processed as described in Example 9 to provide the title compound. ¹H NMR (CD₃OD) δ 2.80 (dd, J=1.7, 16.5 Hz, 1H), 3.18 (dd, J=5.5, 11.7 Hz, 1H), 3.21 (m, 1H), 3.38 (dd, J=3.0, 11.7 Hz, 1H), 3.47 (dd, J=8.1, 11.6 Hz, 1H), 3.55 (dd, J=8.7, 11.6 Hz, 1H), 3.86 (m, 1H), 6.35 (d, J=1.6 Hz, 1H), 7.81 (d, J=9.4 Hz, 1H), 7.92 (s, 1H), 8.10 (m, 2H), 8.73 (s, 1H); MS (ESI) m/z 226 (M+H)⁺.

### EXAMPLE 15

### (cis)-5-(imidazo[1,2-a]pyridin-6-yl)-1,2,3,3a,4,6a-hexahydrocyclopenta[c]pyrrole Hydrochloride

The product from Example 4B and 6-bromoimidazo[1,2-a]pyridine, prepared as described in J. Org. Chem. (1978), 43, 2900-2906, were processed as described in Example 9 to provide the title compound. ¹H NMR (CD₃OD) δ 2.90 (d, J=14.0 Hz, 1H), 3.25 (dd, J=4.4, 11.6 Hz, 1H), 3.32-3.36 (m, 1H), 3.51 (d, J=5.3 Hz, 1H), 3.57 (dd, J=8.1, 11.5 Hz, 1H), 3.98 (m, 1H), 6.44 (s, 1H), 7.59 (dd, J=5.9, 6.7 Hz, 1H), 7.97-8.04 (m, 2H), 8.14 (s, 1H), 9.05 (d, J=7.2 Hz, 1H); MS (ESI) m/z 226 (M+H)⁺.

### EXAMPLE 16

### (cis)-5-(thieno[3,2-b]pyridin-2-yl)-1,2,3,3a,4,6a-hexahydrocyclopenta[c]pyrrole Hydrochloride

The product from Example 4B and 2-iodothieno[3,2-b]pyridine, prepared as described in J. Heterocycl. Chem. (1984), 21, 785-790, were processed as described in Example 9 to provide the title compound. ¹H NMR (CD₃OD) δ 2.94 (ddd, J=2.2, 3.9, 16.4 Hz, 1H), 3.22 (dd, J=5.2, 12.0 Hz, 1H), 3.26-3.34 (m, 1H), 3.36-3.44 (m, 2H), 3.49 (dd, J=8.1, 11.6 Hz, 1H), 3.57 (dd, J=8.6, 11.7 Hz, 1H), 3.92 (tt, J=2.7, 8.1 Hz, 1H), 6.52 (dd, J=2.2, 4.1 Hz, 1H), 7.65 (s, 1H), 7.85 (dd, J=5.6, 8.1 Hz, 1H), 8.84 (dd, J=1.0, 5.6 Hz, 1H), 9.04 (d, J=8.1 Hz, 1H); MS (ESI) m/z 243 (M+H)⁺.

### EXAMPLE 18

### (cis)-5-(1,2,3,3a,4,6a-hexahydrocyclopenta[c]pyrrol-5-yl)-2-thiophenesulfonamide Hydrochloride

The product from Example 4B and 5-bromothiophene-2-sulfonamide (purchased from Fluorochem USA) were processed as described in Example 9 to provide the title compound. ¹H NMR (CD₃OD) δ 2.74 (m, 1H), 3.10-3.19 (m, 2H), 3.22-3.35 (m, 2H), 3.38 (dd, J=8.5, 11.7 Hz, 1H), 3.50 (dd, J=8.7, 11.8 Hz, 1H), 3.79 (m, 1H), 6.04 (br s, 1H), 7.06 (d, J=3.7 Hz, 1H), 7.48 (d, J=3.8 Hz, 1H); MS (ESI) m/z 271 (M+H)⁺.

### EXAMPLE 19

### (cis)-5-(6-methyl-3-pyridinyl)-1,2,3,3a,4,6a-hexahydrocyclopenta[c]pyrrole Dihydrochloride

### Example 19A

### Tert-butyl (cis)-5-(6-methyl-3-pyridinyl)-3,3a,4,6a-tetrahydrocyclopenta[c]pyrrole-2(1H)-carboxylate

The product from Example 4B and 5-bromo-2-methylpyridine, prepared as described in WO 0044755, were processed as described in Example 4C to provide the title compound (50% yield). MS (DCI/NH₃) m/z 301 (M+H)⁺.

### Example 19B

### (cis)-5-(6-methyl-3-pyridinyl)-1,23,3a,4,6a-hexahydrocyclopenta[c]pyrrole Dihydrochloride

The product from Example 19A was processed as described in Example 8B to provide the title compound (88% yield). ¹H NMR (CD₃OD, 400 MHz) δ 2.78 (s, 3H), 2.82 (m, 1H), 3.15-3.27 (m, 2H), 3.29-3.60 (m, 4H), 3.87 (m, 1H), 6.48 (m, 1H), 7.90 (d, J=8.5 Hz, 1H), 8.60 (dd, J=2.0, 8.4 Hz, 1H), 8.75 (d, J=2.1 Hz, 1H); MS (DCI/NH₃) m/z 201 (M+H)⁺; Anal. calculated for C₁₃H₁₆N₂.2HCl: C, 57.15; H, 6.64; N, 10.25. Found: C, 56.88; H, 6.65; N, 10.23.

### EXAMPLE 20

### (cis)-5-(2-methyl-3-pyridinyl)-1,2,3,3a,4,6a-hexahydrocyclopenta[c]pyrrole Hydrochloride

### Example 20A

### Tert-butyl (cis)-5-(2-methyl-3-pyridinyl)-3,3a,4,6a-tetrahydrocyclopenta[c]pyrrole-2(1H)-carboxylate

The product from Example 4B and 3-bromo-2-methylpyridine were processed as described in Example 4C to provide the title compound (19% yield). MS (DCI/NH₃) m/z 301 (M+H)⁺.

### Example 20B

### (cis)-5-(2-methyl-3-pyridinyl)-1,2,3,3 a,4,6a-hexahydrocyclopenta[c]pyrrole Hydrochloride

The product from Example 20A was processed as described in Example 8B to provide the title compound (53% yield). ¹H NMR (CD₃OD, 400 MHz) δ 2.78 (m, 1H), 2.85 (s, 3H), 3.20-3.60 (m, 6H), 3.90 (m, 1H), 6.08 (m, 1H), 7.90 (dd, J=5.7, 7.8 Hz, 1H), 8.49 (d, J=8.1 Hz, 1H), 8.62 (dd, J=1.7, 7.5 Hz, 1H); MS (DCI/NH₃) m/z 201 (M+H)⁺; Anal. calculated for C₁₃H₁₆N₂.2HCl.0.25H₂O: C, 56.23; H, 6.71; N, 10.09. Found: C, 56.40; H, 6.57; N, 9.96.

### EXAMPLE 21

### (cis)-5-(6-chloro-5-fluoro-3-pyridinyl)-1,2,3,3a,4,6a-hexahydrocyclopenta[c]pyrrole Hydrochloride

### Example 21A

### Tert-butyl (cis)-5-(6-chloro-5-fluoro-3-pyridinyl)-3,3a,4,6a-tetrahydrocyclopenta[c]pyrrole-2(1H)-carboxylate

2-Chloro-3-fluoro-5-iodopyridine (0.400 g, 1.08 mmol), prepared as described in J. Org. Chem. (1993), 58, 7832, triphenylarsine (0.026 g, 0.084 mmol) and tris(dibenzylideneacetone)dipalladium(0) (0.019 g, 2 mol %) in anhydrous 1-methyl-2-pyrrolidinone (5 mL) were treated with the product from Example 4B (0.310 g, 1.20 mmol) in 1-methyl-2-pyrrolidinone (2 mL). After stirring at 60° C. for 5 hours, the reaction mixture was allowed to cool to ambient temperature and partitioned between ethyl acetate and 1N aqueous sodium hydroxide. The organic extract was separated, washed with 1N aqueous sodium hydroxide, water, brine, dried over Na₂SO₄, filtered and the filtrate concentrated under reduced pressure. The residue was purified by chromatography (SiO₂, 50% ethyl acetate/hexanes) to provide the title compound (0.226 g, 62%). MS (DCI/NH₃) m/z 339 (M+H)⁺.

### Example 21B

### (cis)-5-(6-chloro-5-fluoro-3-pyridinyl)-1,2,3,3a,4,6a-hexahydrocyclopenta[c]pyrrole Hydrochloride

The product from Example 21A (215 mg, 0.60 mmole) in CH₂Cl₂ (1 mL) and TFA (1 mL) was stirred at ambient temperature for 1 hour and then concentrated under reduced pressure. The residue was purified by chromatography (silica gel; CHCl₃/MeOH/NH₄OH 90:10:1) to provide the free base of the title compound. The free base was dissolved in MeOH and treated with a solution of 1M HCl in diethyl ether to provide 137 mg (77%) of the title compound. ¹H NMR (CD₃OD, 300 MHz) δ 2.76 (m, 1H), 3.18 (m, 2H), 3.30-3.55 (m, 4H), 3.83 (m, 1H), 6.30 (m, 1H), 7.86 (dd, J=2.0, 9.9 Hz, 1H), 8.35 (d, J=2.0 Hz, 1H); MS (DCI/NH₃) m/z 239 (M+H)⁺; Anal. calculated for C₁₂H₁₂ClFN₂.1.5HCl.0.2CH₃OH: C, 48.88; H, 4.81; N, 9.34. Found: C, 49.00; H, 4.53; N, 8.95.

### EXAMPLE 23

### (cis)-5-(6-fluoro-3-pyridinyl)-1,2,3,3a,4,6a-hexahydrocyclopenta[c]pyrrole Hydrochloride

### Example 23A

### Tert-butyl 5-(6-fluoro-3-pyridinyl)-3,3a,4,6a-tetrahydrocyclopenta[c]pyrrole-2(1H)-carboxylate

5-Bromo-2-flouropyridine (0.200 g, 0.538 mmol), triphenylarsine (0.013 g, 0.043 mmol), and tris(dibenzylideneacetone)dipalladium(0) (0.010 g, 2 mol %) in anhydrous 1-methyl-2-pyrrolidinone (2 mL) were treated with the product from Example 4B (0.392 g, 1.05 mmol) in 1-methyl-2-pyrrolidinone (1 mL). The reaction mixture was stirred at 60° C. for 16 hours, allowed to cool to ambient temperature and partitioned between ethyl acetate and 1N aqueous sodium hydroxide. The organic portion was separated, washed with 1N aqueous sodium hydroxide, water, brine, dried over Na₂SO₄, filtered and the filtrate concentrated under reduced pressure. The residue was purified by chromatography (SiO₂, 50% ethyl acetate/hexanes) to provide the title compound (0.126 g, 77%). MS (DCI/NH₃) m/z 305 (M+H)⁺.

### Example 23B

### (cis)-5-(6-fluoro-3-pyridinyl)-1,2,3,3a,4,6a-hexahydrocyclopenta[c]pyrrole Hydrochloride

The product from Example 23A (0.126 g, 0.414 mmol) in methanol (1 mL) was treated with a 1M solution of HCl in diethyl ether (2 mL). After stirring at ambient temperature for 2 hours, the volatiles were evaporated under reduced pressure. The residue was crystallized from ethanol and diethyl ether to provide the title compound (0.067 g, 67%). ¹H NMR (CD₃OD, 300 MHz) δ 2.76 (m, 1H), 3.13-3.21 (m, 2H), 3.36-3.55 (m, 4H), 3.81 (m, 1H), 6.16 (m, 1H), 7.07 (ddd, J=0.7, 2.7, 8.8 Hz, 1H), 8.09 (ddd, J=2.7, 7.8, 8.6 Hz, 1H); MS (DCI/NH₃) m/z 205 (M+H)⁺.

### EXAMPLE 24

### (cis)-5-(1,2,3,3a,4,6a-hexahydrocyclopenta[c]pyrrol-5-yl)-2-thiophenecarboximidamide Hydrochloride

The product from Example 4B and the free base of 5-chloro-2-thiophenecarboximidamide monohydrochloride, prepared as descibed in EP 819690, were processed as described in Example 9 to provide the title compound. ¹H NMR (CD₃OD) δ 2.74 (br d, J=12.6 Hz, 1H), 3.14-3.24 (m, 2H), 3.27-3.41 (m, 2H), 3.38 (m, 1H), 3.84 (m, 1H), 6.21 (br s, 1H), 7.29 (d, J=4.1 Hz, 1H), 7.88 (d, J=4.0 Hz, 1H); MS (ESI) m/z 234 (M+H)⁺.

### EXAMPLE 25

### (cis)-5-(2-methyl-2H-tetraazol-5-yl)-1,2,3,3a,4,6a-hexahydrocyclopenta[c]pyrrole Hydrochloride

The product from Example 4B and 5-bromo-2-methyl-2H-tetrazole, prepared as described in Can. J. Chem., (1973), 51, 2315-2322, were processed as described in Example 9 to provide the title compound. ¹H NMR (CD₃OD) δ 2.94 (ddd, J=2.2, 4.1, 17.2 Hz, 1H), 3.15 (dd, J=5.3, 11.9 Hz, 1H), 3.18-3.24 (m, 1H), 3.37 (dd, J=3.0, 12.0 Hz, 1H), 3.45 (dd, J=8.0, 12.0 Hz, 1H), 3.52 (dd, J=8.7, 11.9 Hz, 1H), 3.85 (tt, J=2.6, 7.9 Hz, 1H), 4.35 (s, 3H), 6.51 (dd, J=2.2, 4.1 Hz, 1H); MS (ESI) m/z 192 (M+H)⁺.

### EXAMPLE 26

### (cis)-5-(thieno[2,3-b]pyridin-5-yl)-1,2,3,3a,4,6a-hexahydrocyclopenta[c]pyrrole Hydrochloride

The product from Example 4B and 5-bromothieno[2,3-b]pyridine, prepared as described in J. Heterocycl. Chem, (1968), 5, 773-778, were processed as described in Example 9 to provide the title compound. ¹H NMR (CD₃OD) δ 2.87 (dd, J=2.9, 16.4 Hz, 1H), 3.20 (dd, J=5.0, 11.9 Hz, 1H), 3.26 (tt, J=2.2, 8.3 Hz, 1H), 3.32-3.37 (m, 1H), 3.42 (dd, J=2.7, 11.7 Hz, 1H), 3.46 (dd, J=8.0, 11.7 Hz, 1H), 3.55 (dd, J=8.9, 11.7 Hz, 1H), 3.87 (m, 1H), 6.36 (d, J=1.9 Hz, 1H), 7.50 (d, J=5.9 Hz, 1H), 7.87 (d, J=5.9 Hz, 1H), 8.56 (d, J=1.9 Hz, 1H), 8.86 (d, J=1.9 Hz, 1H); MS (ESI) m/z 243 (M+H)⁺.

### EXAMPLE 27

### (cis)-5-(imidazo[1,2-a]pyridin-7-yl)-1,2,3,3a,4,6a-hexahydrocyclopenta[c]pyrrole Hydrochloride

The product from Example 4B and 7-chloroimidazo[1,2-a]pyridine, prepared as described in J. Heterocycl. Chem, (1965), 2, 53-62, were processed as described in Example 9 to provide the title compound. ¹H NMR (CD₃OD) δ 2.85 (dd, J=1.6, 16.5 Hz, 1H), 3.19 (dd, J=5.5, 12.0 Hz, 1H), 3.22-3.28 (m, 1H), 3.35-3.37 (m, 1H), 3.40 (dd, J=3.1, 12.2 Hz, 1H), 3.50 (dd, J=8.3, 11.7 Hz, 1H), 3.57 (dd, J=9.1, 11.6 Hz, 1H), 3.90 (m, 1H), 6.61 (d, J=1.6 Hz, 1H), 7.71-7.74 (m, 2H), 8.00 (d, J=2.2 Hz, 1H), 8.17 (d, J=1.6 Hz, 1H), 8.71 (d, J=7.2 Hz, 1H); MS (ESI) m/z 226 (M+H)⁺.

### EXAMPLE 28

### (cis)-5-(2-pyridinyl)-1,2,3,3a,4,6a-hexahydrocyclopenta[c]pyrrole Hydrochloride

The product from Example 4B and 2-bromopyridine (purchased from Aldrich) were processed as described in Example 9 to provide the title compound. ¹H NMR (CD₃OD) δ 2.92 (dd, J=1.9, 16.5 Hz, 1H), 3.22 (dd, J=5.5, 12.0 Hz, 1H), 3.26-3.29 (m, 1H), 3.38-3.41 (m, 1H), 3.44 (dd, J=3.3, 12.3 Hz, 1H), 3.53 (dd, J=8.6, 12.0 Hz, 1H), 3.57 (dd, J=8.6, 12.0 Hz, 1H), 3.96 (tt, J=2.7, 8.2 Hz, 1H), 6.87 (d, J=1.6 Hz, 1H), 7.92 (t, J=6.7 Hz, 1H), 8.14 (d, J=8.4 Hz, 1H), 8.53 (dt, J=4.0, 11.1 Hz, 1H), 8.71 (dd, J=0.9, 5.9 Hz, 1H); MS (ESI) m/z 187 (M+H)⁺.

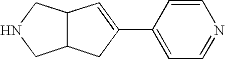

### EXAMPLE 29

### (cis)-5-(4-pyridinyl)-1,2,3,3a,4,6a-hexahydrocyclopenta[c]pyrrole Hydrochloride

The product from Example 4B and the free base of 4-bromopyridine hydrochloride (purchased from Aldrich) were processed as described in Example 9 to provide the title compound. ¹H NMR (CD₃OD) δ 2.86 (dd, J=1.7, 16.7 Hz, 1H), 3.19 (dd, J=5.3, 11.6 Hz, 1H), 3.22-3.27 (m, 1H), 3.35-3.40 (m, 1H), 3.42 (dd, J=3.1, 11.6 Hz, 1H), 3.52 (dd, J=8.6, 12.0 Hz, 1H), 3.56 (dd, J=8.7, 11.6 Hz, 1H), 3.93 (m, 1H), 6.87 (dd, J=2.2, 3.7 Hz, 1H), 8.08 (d, J=6.6 Hz, 2H), 8.75 (d, J=6.4 Hz, 2H); MS (ESI) m/z 187 (M+H)⁺.

### EXAMPLE 30

### (cis)-5-(5-nitro-1,3-thiazol-2-yl)-1,2,3,3a,4,6a-hexahydrocyclopenta[c]pyrrole Hydrochloride

The product from Example 4B and 2-bromo-5-nitrothiazole (purchased from Aldrich) were processed as described in Example 9 to provide the title compound. ¹H NMR (CD₃OD) δ 2.85 (dd, J=1.9, 16.9 Hz, 1H), 3.16 (dd, J=5.2, 12.0 Hz, 1H), 3.20-3.27 (m, 1H), 3.33-3.35 (m, 1H), 3.38 (dd, J=3.1, 11.9 Hz, 1H), 3.45 (dd, J=8.3, 12.0 Hz, 1H), 3.51 (dd, J=8.4, 11.6 Hz, 1H), 3.87 (m, 1H), 6.67 (dd, J=2.2, 4.1 Hz, 1H), 8.61 (s, 1H); MS (ESI) m/z 238 (M+H)⁺.

### EXAMPLE 31

### (cis)-5-(6-methyl-2-pyridinyl)-1,2,3,3a,4,6a-hexahydrocyclopenta[c]pyrrole Hydrochloride

The product from Example 4B and 2-bromo-6-methylpyridine (purchased from Aldrich) were processed as described in Example 9 to provide the title compound. ¹H NMR (CD₃OD) δ 2.59 (s, 3H), 2.92 (d, J=16.2 Hz, 1H), 3.30-3.42 (m, 3H), 3.47-3.65 (m, 3H), 3.96 (m, 1H), 6.40 (d, J=1.6 Hz, 1H), 7.92 (t, J=6.9 Hz, 1H), 8.49 (d, J=7.8 Hz, 1H), 8.64 (d, J=5.7 Hz, 1H); MS (ESI) m/z 201 (M+H)⁺.

### EXAMPLE 32

### (cis)-5-(1,3,4-thiadiazol-2-yl)-1,2,3,3a,4,6a-hexahydrocyclopenta[c]pyrrole Hydrochloride

The product from Example 4B and 2-bromo-1,3,4-thiadiazole, prepared as described in Chem. Ber., (1956), 56, 1534-1543, were processed as described in Example 9 to provide the title compound. ¹H NMR (CD₃OD) δ 2.86 (m, 1H), 3.20 (dd, J=4.3, 11.8 Hz, 1H), 3.30-3.60 (m, 5H), 3.84 (m, 1H), 6.52 (br s, 1H), 9.40 (s, 1H); MS (ESI) m/z 194 (M+H)⁺.

### EXAMPLE 34

### (endo)-5-(3-pyridinyl)octahydrocyclopenta[c]pyrrole bis(4-methylbenzenesulfonate)

### Example 34A

### (endo)-5-(3-pyridinyl)octahydrocyclopenta[c]pyrrole

The product from Example 1G (0.077 g, 0.263 mmol) in ethanol was treated with hydrogen gas (1 atm) in the presence of Pd/C (0.008 g, 10 wt. %) and stirred at ambient temperature for 4 hours. The reaction vessel was evacuated, purged with nitrogen and the catalyst was removed by filtration through diatomaceous earth. The filtrate was concentrated under reduced pressure and the residue was purified by chromatography (SiO₂, CHCl₃/MeOH/NH₄OH, 90:10:1) to provide the title compound (0.036 g, 39%). MS (DCI/NH₃) m/z 189 (M+H)⁺.

### Example 34B

### (endo)-5-(3-pyridinyl)octahydrocyclopenta[c]pyrrole bis(4-methylbenzenesulfonate)

The product from Example 34A (0.020 g, 0.0795 mmol) in methanol was treated with p-toluenesulfonic acid (0.030 g, 0.159 mmol) and stirred at ambient temperature for 5 minutes. The volatiles were evaporated under reduced pressure and the residue was crystallized from ethanol and diethyl ether to provide the title compound (0.015 g, 30%). ¹H NMR (CD₃OD, 300 MHz) δ 1.67 (m, 2H), 2.37 (s, 6H), 2.52 (m, 2H), 3.11 (m, 2H), 3.29-3.40 (m, 4H), 7.23 (d, J=8.1 Hz, 4H), 7.70 (d, J=8.1 Hz, 4H), 8.03 (dd, J=5.7, 8.1 Hz, 1H), 8.63 (d, J=8.5 Hz, 1H), 8.72 (d, J=5.4 Hz, 1H), 8.82 (s, 1H); MS (DCI/NH₃) m/z 189 (M+H)⁺.

### EXAMPLE 35

### (cis)-5-(6-chloro-3-pyridinyl)octahydrocyclopenta[c]pyrrol-5-ol

The product from Example 1E (0.044 g, 0.130 mmol) in methanol (1 mL) was treated with a 1M solution of HCl in diethyl ether (1 mL). After stirring at ambient temperature for 2 hours, the volatiles were evaporated under reduced pressure. The residue was crystallized from ethanol and diethyl ether to provide the title compound (0.031 g, 87%). ¹H NMR (CD₃OD, 300 MHz) δ 2.08 (d, J=14.0 Hz, 2H), 2.38 (m, 1H), 2.41 (m, 1H), 3.24 (m, 2H), 3.41 (d, J=5.5 Hz, 4H), 7.44 (d, J=8.5 Hz, 1H), 7.93 (dd, J=2.6, 8.5 Hz, 1H), 8.49 (d, J=2.6 Hz, 1H); MS (DCI/NH₃) m/z 239 (M+H)⁺.

### EXAMPLE 36

### (endo)-5-(6-chloro-3-pyridinyl)octahydrocyclopenta[c]pyrrole Hydrochloride

### Example 36A

### (endo)-5-(6-chloro-3-pyridinyl)octahydrocyclopenta[c]pyrrole

### (exo)-5-(6-chloro-3-pyridinyl)octahydrocyclopenta[c]pyrrole

The product from Example 1E (0.336 g, 0.994 mmol) in acetonitrile (5 mL) at 0° C. was treated with 4-(dimethylamino)pyridine (0.672 g, 1.49 mmol) and methyl oxalyl chloride (0.32 mL, 1.19 mmol). The reaction mixture was allowed to warm to room temperature, diluted with ethyl acetate, washed with saturated aqueous sodium bicarbonate (20 mL), water (20 mL), brine (20 mL), dried over Na₂SO₄, filtered and the filtrate was concentrated under reduced pressure. The residue was dissolved in toluene (5 mL) and treated with tributyltin hydride (0.401 mL, 1.49 mmol) and AIBN (0.024 g, 0.149 mmol). The reaction mixture was stirred at 100° C. for 3 hours, allowed to cool to room temperature and concentrated under reduced pressure. The residue was purified by chromatography (SiO₂, 30% ethyl acetate/hexane) to provide 0.019 g (6%) of the exo-isomer and 0.094 g (29%) of the endo-isomer. exo-isomer: ¹H NMR (CDCl₃, 300 MHz) δ 1.47 (s, 9H), 1.86 (m, 2H), 1.99 (m, 2H), 2.90 (m, 2H), 3.14-3.30 (m, 3H), 3.65 (m, 2H), 7.24 (d, J=38.6 Hz, 1H), 7.48 (dd, J=2.4, 8.1 Hz, 1H), 8.26 (d, J=2.5 Hz, 1H); MS (DCI/NH₃) m/z 323, 325 (M+H)⁺. endo-isomer: ¹H NMR (CDCl₃, 300 MHz) δ 1.47 (s, 9H), 1.48 (m, 2H), 2.33 (m, 2H), 2.75 (m, 2H), 3.09 (m, 1H), 3.35 (m, 2H), 3.45 (m, 2H), 7.24 (d, J=8.1 Hz, 1H), 7.51 (dd, J=2.4, 8.1 Hz, 1H). 8.25 (d, J=2.4 Hz); MS (DCI/NH₃) m/z 323, 325 (M+H)⁺.

### Example 36B

### (endo)-5-(6-chloro-3-pyridinyl)octahydrocyclopenta[c]pyrrole Hydrochloride

The endo-isomer from Example 36A (0.094 g, 0.292 mmol) in methanol (1 mL) was treated with a 1M solution of HCl in diethyl ether (1 mL). After stirring at ambient temperature for 2 hours, the volatiles were evaporated under reduced pressure. The residue was crystallized from ethanol and diethyl ether to provide the title compound (0.062 g, 32%). ¹H NMR (CD₃OD, 300 MHz) δ 1.51 (m, 2H), 2.47 (m, 2H), 3.07 (m, 2H), 3.13-3.38 (m, 5H), 7.40 (d, J=8.1 Hz, 1H), 7.77 (dd, J=2.7, 8.1 Hz, 1H), 8.28 (d, J=2.7, 1H); MS (DCI/NH₃) m/z 223 (M+H)⁺.

### EXAMPLE 37

### (exo)-5-(6-chloro-3-pyridinyl)octahydrocyclopenta[c]pyrrole Hydrochloride

The exo-isomer from Example 36A (0.019 g, 0.059 mmol) in methanol (1 mL) was treated with a 1M solution of HCl in diethyl ether (1 mL). After stirring at ambient temperature for 2 hours, the volatiles were evaporated under reduced pressure. The residue was crystallized from ethanol and diethyl ether to provide the title compound (0.011 g, 72%). ¹H NMR (CD₃OD, 300 MHz) δ 1.99 (m, 4H), 2.95 (m, 2H), 3.06 (m, 2H), 3.40 (m, 1H), 3.61 (m, 2H), 7.41 (d, J=8.5 Hz, 1H), 7.77 (dd, J=2.6, 8.5 Hz, 1H), 8.29 (d, J=2.6 Hz, 1H); MS (DCI/NH₃) m/z 223 (M+H)⁺.

### EXAMPLE 38

### (cis)-5-(3-pyridinyl)-1,2,3,3a,4,6a-hexahydrocyclopenta[b]pyrrole Fumarate

### (cis)-5-(3-pyridinyl)-12,3,3a,6,6a-hexahydrocyclopenta[b]pyrrole Fumarate

### Example 38A

### benzyl 5-oxohexahydrocyclopenta[b]pyrrole-1 (2H)-carboxylate

Benzyl 6,6-dichloro-5-oxohexahydrocyclopenta[b]pyrrole-1(2H)-carboxylate (3.87 g, 11.8 mmol), prepared as descibed in Tetrahedron Lett. (1997), 38, 1869-1872, in MeOH (60 mL) at 0° C. was treated with solid ammonium chloride (6.32 g, 118 mmol) followed by zinc duct (Aldrich; 3.09 g, 47.2 mmol). After stirring at room temperature for 1 hour, the reaction mixture was filtered and the filtrate concentrated under reduced pressure. The residue was purified by chromatography (SiO₂, 40% EtOAc/hexanes) to provide 2.57 g (84%) of the title compound. MS (DCI/NH₃) mn/z 260 (M+H)⁺.

### Example 38B

### benzyl 5-{[(trifluoromethyl)sulfonyl]oxy}-3,3a,6,6a-tetrahydrocyclopenta[b]pyrrole-1 (2H)-carboxylate

### benzyl 5-{[(trifluoromethyl)sulfonyl]oxy}-3,3a,4,6a-tetrahydrocyclopenta[b]pyrrole-1 (2H)-carboxylate

The product from Example 38A (1.56 g, 6.02 mmol) in THF (30 mL) at −78° C. was treated with potassium bis(trimethylsilyl)amide (0.5M solution in toluene, 14.4 mL, 7.22 mmol). The reaction mixture was stirred at −78° C. for 30 minutes and then a solution of N-phenyltrifluoromethanesulfonimide (2.58 g, 7.22 mmol) in THF (5 mL) was added dropwise. The reaction mixture was stirred at −78° C. for 1 hour and then quenched by the addition of water. The mixture was warmed to ambient temperature and extracated with CH₂Cl₂. The organic extract was washed with saturated aqueous NH₄Cl, brine, dried (Na₂SO₄), filtered and the filtrate concentrated under reduced pressure. The residue was purified by chromatography (SiO₂, 20% EtOAc/hexanes) to provide 1.84 g (78%) of a 4:1 mixture of the title compounds. MS (DCI/NH₃) m/z 392 (M+H)⁺.

### Example 38C

### benzyl 5-(trimethylstannyl)-3,3a,6,6a-tetrahydrocyclopenta[b]pyrrole-1 (2H)-carboxylate

### benzyl 5-(trimethylstannyl)-3,3a,4,6a-tetrahydrocyclopenta[b]pyrrole-1(2H)-carboxylate

The product mixture of Example 38B (260 mg, 0.664 mmol) in THF (3.3 mL) was treated with hexamethylditin (218 mg, 0.664 mmol), lithium chloride (84 mg, 2.0 mmol) and tetrakis(triphenylphosphine)palladium(0) (15 mg, 0.0013 mmol). The resulting mixture was stirred at 60° C. for 14 hours, allowed to cool to room temperature and filtered through Celite (diethyl ether wash). The filtrate was washed with 1N aqueous sodium hydroxide (2×), water, brine, dried (Na₂SO₄), filtered and the filtrate concentrated under reduced pressure. The residue was purified by chromatography (SiO₂, 15% ethyl acetate/hexanes) to provide 160 mg (57%) of a 4:1 mixture of the title compounds. MS (DCI/NH₃) m/z 423, 425 (M+H)⁺.

### Example 38D

### benzyl 5-(3-pyridinyl)-3,3a,6,6a-tetrahydrocyclopenta[b]pyrrole-1(2H)-carboxylate

### benzyl 5-(3-pyridinyl)-3,3a,4,6a-tetrahydrocyclopenta[b]pyrrole-1 (2H)-carboxylate

The mixture of Example 38C (140 mg, 0.332 mmol) and 3-bromopyridine (0.042 mL, 0.43 mmol) were processed as described in Example 4C. Purification by chromatography (SiO₂, 30% EtOAc/hexane) provided 90 mg (85%) of a 4:1 mixture of the title compounds. MS (DCI/NH₃) m/z 321 (M+H)⁺.

### Example 38E

### (cis)-5-(3-pyridinyl)-1,2,3,3a,4,6a-hexahydrocyclopenta[b]pyrrole Fumarate

### (cis)-5-(3-pyridinyl)-1,2,3,3a,6,6a-hexahydrocyclopenta[b]pyrrole Fumarate

Iodotrimethylsilane (43 μL, 0.30 mmol) was added to a 0° C. solution of the mixture from Example 38D (80 mg, 0.25 mmol) in 2.5 mL of acetonitrile. The solution was stirred for 3 hours at 0° C. then concentrated under reduced pressure. The residue was passed through a plug of silica gel (10% MeOH/CH₂Cl₂ wash) to provide 38 mg (82%) of a mixture of the free amines. The free amines were dissolved in MeOH and fumaric acid was added (23 mg, 0.19 mmol). After 30 minutes, a few drops of diethyl ether were added causing formation of a precipitate that was then collected by filtration. The solid was dried under reduced pressure to provide 42 mg of a 4:1 mixture of the title compounds. ¹H NMR for major isomer (D₂O, 300 MHz) δ 1.95 (m, 1H), 2.30 (m, 1H), 2.77 (m, 1H), 3.13-3.49 (m, 5H), 6.36 (m, 1H), 6.60 (s, 2H), 7.77 (dd, J=2.6, 8.5 Hz, 1H), 8.34 (m, 1H), 8.60 (m, 1H), 8.83 (m, 1H); MS (DCI/NH₃) m/z 187 (M+H)⁺. Peaks for the minor isomer in the ¹H NMR are obscured by the major isomer, except for the olefinic proton (multiplet at 6.45 ppm) that allows for a rough determination of the product ratios.

### EXAMPLE 39

### (cis)-5-(5-bromo-3-pyridinyl)-1,2,3,3a,4,6a-hexahydrocyclopenta[c]pyrrole Fumarate

### Example 39A

### Tert-butyl (cis)-5-(5-bromo-3-pyridinyl)-3,3a,4,6a-tetrahydrocyclopenta[c]pyrrole-2(1H)-carboxylate

3,5-Dibromopyridine (0.656 g, 2.77 mmol) and the product from Example 4B (1.03 g, 2.77 mmol) were processed as described in Example 4C to provide 477 mg (47%) of the title compound. MS (DCI/NH₃) m/z 365, 367 (M+H)⁺.

### Example 39B

### (cis)-5-(5-bromo-3-pyridinyl)-1,2,3,3a,4,6a-hexahydrocyclopenta[c]pyrrole Fumarate

The product from Example 39A (0.110 g, 0.302 mmol) in CH₂Cl₂ (mL) was treated with TFA (1 mL). After stirring for 30 minutes, the solution was concentrated and then diluted with 10% NH₄OH/MeOH. This was concentrated under reduced pressure and the residue passed through a pad of silica gel (90:10:1 CHCl₃:MeOH:NH₄OH eluent). The filtrate was concentrated to afford the free base of the title compound which was diluted with MeOH and treated with fumaric acid (0.033 g 0.28 mmol) to afford the title compound (77 mg, 67%). ¹H NMR (CD₃OD, 300 MHz) δ 2.75 (m, 1H), 3.11-3.21 (m, 2H), 3.35-3.59 (m, 4H), 3.89 (m, 1H), 6.29 (m, 1H), 6.65 (s, 2H), 8.12 (dd, J=2.0, 2.1 Hz, 1H), 8.55 (d, J=2.4 Hz, 1H), 8.63 (d, J=2.4 Hz, 1H); MS (DCI/NH₃) m/z 265, 267 (M+H)⁺; Anal. calculated for C₁₂H₁₄N₂.C₄H₄O₄: C, 50.41; H, 4.49; N, 7.35. Found: C, 50.51; H, 4.53; N, 7.33.

### EXAMPLE 40

### (cis)-5-(5-vinyl-3-pyridinyl)-1,2,3,3a,4,6a-hexahydrocyclopenta[c]pyrrole Fumarate

### Example 40A

### Tert-butyl (cis)-5-(5-vinyl-3-pyridinyl)-3,3a,4,6a-tetrahydrocyclopenta[c]pyrrole-2(1H)-carboxylate

Tributyl(vinyl)tin (0.656 g, 2.77 mmol), tetrakis(triphenylphosphine)palladium(0) (0.026 g, 0.23 mmol), in anhydrous toluene were treated with the product from Example 39A (1.03 g, 2.77 mmol). The reaction mixture was stirred for 3 days at 100° C. After cooling to ambient temperature, the volatiles were removed under reduced pressure and the residue was purified by flash chromatography to afford the title compound (279 mg, 93%). MS (DCI/NH₃) m/z 312 (M+H)⁺.

### Example 40B

### (cis)-5-(5-vinyl-3-pyridinyl)-1,2,3,3 a,4,6a-hexahydrocyclopenta[c]pyrrole Fumarate

The product from Example 40A (0.279 g, 0.894 mmol) was processed as described in Example 39B to provide the title compound (0.168 g, 57%). ¹H NMR (CD₃OD, 300 MHz) δ 2.80 (m, 1H), 3.14-3.54 (m, 6H), 3.83 (m, 1H), 5.45 (d, J=11.2 Hz, 1H), 5.98 (d, J=18.0 Hz, 1H), 6.27 (m, 1H), 6.66 (s, 2H), 6.80 (dd, J=1.2,18.0 Hz, 1H), 7.99 (dd, J=2.0, 2.0 Hz, 1H), 8.49 (d, J=2.0 Hz, 1H), 8.55 (d, J=2.0 Hz, 1H); MS (DCI/NH₃) m/z 213 (M+H)⁺; Anal. calculated for C₁₄H₁₆N₂.C₄H₄O₄: C, 65.84; H, 6.14; N, 8.53. Found: C, 65.64; H, 6.07; N, 8.30.

### EXAMPLE 41

### (cis)-5-(1,2,3,3a,4,6a-hexahydrocyclopenta[c]pyrrol-5-yl)nicotinonitrile

### Example 41A

### Tert-butyl (cis)-5-(5-cyano-3-pyridinyl)-3,3a,4,6a-tetrahydrocyclopenta[c]pyrrole-2(1H)-carboxylate

3-Bromo-5-cyanopyridine (0.251 g, 1.37 mmol) and the product from Example 4B (0.510 g, 137 mmol) were processed as described in Example 4C to provide 232 mg (54%) of the title compound. MS (DCI/NH₃) m/z 312 (M+H)⁺.

### Example 41B

### (cis)-5-(1,2,3,3a,4,6a-hexahydrocyclopenta[c]pyrrol-5-yl)nicotinonitrile fumarate

The product from Example 41A (0.091 g, 0.29 mmol) was processed as described in Example 39B to provide the title compound (0.059 g, 62%). ¹H NMR (CD₃OD, 300 MHz) δ 2.77 (m, 1H), 3.12-3.33 (m, 2H), 3.28-3.54 (m, 4H), 3.83 (m, 1H), 6.37 (m, 1H), 6.65 (s, 2H), 8.28 (dd, J=2.0, 2.0 Hz, 1H), 8.80 (d, J=1.70 Hz, 1H), 8.83 (d, J=2.0 Hz, 1H); MS (DCI/NH₃) m/z 212 (M+H)⁺; Anal. calculated for C₁₄H₁₆N₂.C₄H₄O₄: C, 62.38; H, 5.23; N, 12.84. Found: C, 62.52; H, 5.27; N, 13.01.

### EXAMPLE 42

### (cis)-5-(6-chloro-3-pyridinyl)-1,2,3,3,3a,4,6a-hexahydrocyclopenta[c]pyrrole Hydrochloride

### Example 42A

### (+)-tert-butyl (cis)-5-(6-chloro-3-pyridinyl)-3,3a,4,6a-tetrahydrocyclopenta[c]pyrrole-2(1H)-carboxylate and

### (−)-tert-butyl (cis)-5-(6-chloro-3-pyridinyl)-3,3a,4,6a-tetrahydrocyclopenta[c]pyrrole-2(1H)-carboxylate

The racemic product from Example 1F was separated into its individual enantiomers on a Chiralpak AS column (4.6 mm×250 mm) using 98:2 hexane:ethanol as eluent. The more mobile enantiomer, retention time=12.54 minutes, [a]D-46.1 (c 2.0, CH₂Cl₂) The less mobile enantiomer, retention time=17.66 minutes [α]_{D} 48.8 (c 2.0, CH₂Cl₂).

### Example 42B

### (cis)-5-(6-chloro-3-pyridinyl)-1,2,3,3a,4,6a-hexahydrocyclopenta[c]pyrrole Hydrochloride

The more mobile enantiomer, (−)-tert-Butyl (cis)-5-(6-chloro-3-pyridinyl)-3,3a,4,6a-tetrahydrocyclopenta[c]pyrrole-2(1H)-carboxylate, from Example 42A was processed as described in Example 1G to afford the title compound. ¹H NMR (CD₃OD, 300 MHz) δ 2.75 (m, 1H), 3.12-3.22 (m, 2H), 3.32-3.55 (m, 4H), 3.82 (m, 1H), 6.24 (m, 1H), 7.45 (d, J=8.5 Hz, 1H), 7.94 (dd, J=2.4, 8.5 Hz, 1H), 8.47 (d, J=2.1 Hz, 1H); MS (DCI/NH₃) m/z 221, 223 (M+H)⁺.

### EXAMPLE 43

### (cis)-5-(6-chloro-3-pyridinyl)-1,2,3,3a,4,6a-hexahydrocyclopenta[c]pyrrole Hydrochloride

The less mobile enantiomer, (+)-tert-Butyl (cis)-5-(6-chloro-3-pyridinyl)-3,3a,4,6a-tetrahydrocyclopenta[c]pyrrole-2(1H)-carboxylate, from Example 42A was processed as described in Example 1G to afford the title compound. ¹H NMR (CD₃OD, 300 MHz) δ 2.75 (m, 1H), 3.12-3.22 (m, 2H), 3.32-3.55 (m, 4H), 3.82 (m, 1H), 6.24 (m, 1H), 7.45 (d, J=8.5 Hz, 1H), 7.94 (dd, J=2.5, 8.5 Hz, 1H), 8.47 (d, J=2.4 Hz, 1H); MS (DCI/NH₃) m/z 221, 223 (M+H)⁺.

### EXAMPLE 44

### (cis)-6-(6-chloro-3-pyridinyl)-1,2,3,3a,46a-hexahydrocyclopenta[c]pyrrole Fumarate

### Example 44A

### Tert-butyl (cis)-4-oxohexahydrocyclopenta[c]pyrrole-2(1H)-carboxylate

(cis)-2-Benzylhexahydrocyclopenta[c]pyrrol-4(1H)-one, prepared as described in Heterocycles (1988), 27(3), 643-644, and di-tert-butyl-dicarbonate in methanol were treated with hydrogen gas in the presence of palladium hydroxide (20 wt. %). After completion of the reaction, the reaction vessel was purged with nitrogen and the catalyst removed by filtration. The filtrate was concentrated under reduced pressure to provide the title compound. ¹H NMR (CDCl₃, 300 MHz) δ 1.45 (s, 9H), 1.87 (m, 1H), 2.16 (m, 1H), 2.34-2.39 (m, 2H), 2.74 (m, 1H), 3.03 (m, 1H), 3.15 (br m, 1H), 3.42-3.74 (m, 3H).

### Example 44B

### Tert-butyl (cis)-6-{[(trifluoromethyl)sulfonyl]oxy}-3,3a,4,6a-tetrahydrocyclopenta[c]pyrrole-2(1H)-carboxylate

The product from Example 44A (1.78 g, 7.88 mmol) was processed as described in Example 4A to afford the title compound (1.57 g, 56%). MS (DCI/NH₃) m/z 358 (M+H)⁺, 375 (M+NH₄)⁺.

### Example 44C

### Tert-butyl (cis)-6-(trimethylstannyl)-3,3a,4,6a-tetrahydrocyclopenta[c]pyrrole-2(1H)-carboxylate

The product from Example 44B (0.303 g, 0.848 mmol) was processed as described in Example 4B to afford the title compound (0.225 g, 71%). MS (DCI/NH₃) m/z 372, 374 (M+H)⁺.

### Example 44D

### Tert-butyl (cis)-6-(6-chloro-3-pyridinyl)-3,3a,4,6a-tetrahydrocyclopenta[c]pyrrole-2(1H)-carboxylate

2-Chloro-5-iodopyridine and the product from Example 44C were processed as described in Example 4C to afford the title compound. MS (DCI/NH₃) m/z 321, 323 (M+H)⁺.

### Example 44E

### (cis)-6-(6-chloro-3-pyridinyl)-1,2,3,3a,4,6a-hexahydrocyclopenta[c]pyrrole Fumarate

The product from Example 44D was processed as described in Example 39B to afford the title compound: ¹H NMR (CD₃OD, 300 MHz) δ 2.50 (m, 1H), 2.96 (m, 1H), 3.07-3.18 (m, 2H), 3.49-3.62 (m, 2H), 4.10 (m, 1H), 6.39 (m, 1H), 7.46 (dd, J=0.7, 8.1 Hz, 1H), 7.91 (dd, J=2.5, 8.5 Hz, 1H), 8.44 (d, J=2.4 Hz, 1H); MS (DCI/NH₃) m/z 221, 223 (M+H)⁺; Anal. calculated for C₁₂H₁₃ClN₂.C₄H₄O₄: C, 57.06; H, 5.09; N, 8.32. Found: C, 57.15; H, 5.33; N, 8.36.

### EXAMPLE 45

### (cis)-4-(3-pyridinyl)-octahydrocyclopenta[c]pyrrol-4-ol dihydrochloride

### Example 45A

### tert-butyl (cis)-4-hydroxy-4-(3-pyridinyl)-hexahydrocyclopenta[c]pyrrole-2(1H)-carboxylate

3-Bromopyridine (4.36 g, 27.6 mmol) and the product from Example 44A (2.07 g, 9.19 mmol) were processed as described in Example 1E to afford the title compound (0.743 g, 27%). MS (DCI/NH₃) m/z 305 (M+H)⁺

### Example 45B

### (cis)-4-(3-pyridinyl)-octahydrocyclopenta[c]pyrrol-4-ol Dihydrochloride

The product from Example 45A was processed as described in Example 35 to afford the title compound. ¹H NMR (CD₃OD, 300 MHz) δ 1.92 (m, 1H), 2.17 (m, 1H), 2.32-2.51 (m, 2H), 2.98-3.14 (m, 2H), 3.23-3.43 (m, 3H), 3.49 (d, J=11.2 Hz, 1H), 8.11 (dd, J=5.9, 8.4 Hz, 1H), 8.79-8.82 (m, 2H), 9.02 (d, J=1.4 Hz, 1H); MS (DCI/NH₃) m/z 205 (M+H)⁺.

### EXAMPLE 45

### (cis)-4-(3-pyridinyl)-octahydrocyclopenta[c]pyrrol-4-ol Dihydrochloride

### Example 45A

### Tert-butyl-4-hydroxy-4-(3-pyridinyl)-hexahydrocyclopenta[c]pyrrole-2(1H)-carboxylate

3-Bromopyridine (4.36 g, 27.6 mmol) and the product from Example 44A (2.07 g, 9.19 mmol) were processed as described in Example 1E to afford the title compound (0.743 g, 27%). MS (DCI/NH₃) m/z 305 (M+H)⁺.

### Example 45B

### (cis)-4-(3-pyridinyl)-octahydrocyclopenta[c]pyrrol-4-ol Dihydrochloride

The product from Example 45A was processed as described in Example 35 to afford the title compound. ¹H NMR (CD₃OD, 300 MHz) δ 1.92 (m, 1H), 2.17 (m, 1H), 2.32-2.51 (m, 2H), 2.98-3.14 (m, 2H), 3.23-3.43 (m, 3H), 3.49 (d, J=11.2 Hz, 1H), 8.11 (dd, J=5.9, 8.4 Hz, 1H), 8.79-8.82 (m, 2H), 9.02 (d, J=1.4 Hz, 1H); MS (DCI/NH₃) m/z 205 (M+H)⁺.

### EXAMPLE 46

### (endo)-4-(6-chloro-3-pyridinyl)octahydrocyclopenta[c]pyrrole 4-methylbenzenesulfonate

### Example 46A

### (cis)-tert-butyl-4-hydroxy-4-(6-chloro-3-pyridinyl)-hexahydrocyclopenta[c]pyrrole-2(1H)-carboxylate

5-Bromo-2-chloropyridine (1.82 g, 9.46 mmol) and the product from Example 44A (1.64 g, 7.28 mmol) were processed as described in Example 1E to afford the title compound (1.25 g, 51%). MS (DCI/NH₃) m/z 339,341 (M+H)⁺.

### Example 46B

### (endo)-tert-butyl-4-(6-chloro-3-pyridinyl)octahydrocyclopenta[c]pyrrole Carboxylate

### (exo)-tert-butyl-4-(6-chloro-3-pyridinyl)octahydrocyclopenta[c]pyrrole Carboxylate

The product from Example 46A (0.250 g, 0.738 mmol) was processed as described in Example 36A. The residue was purified by chromatography (SiO₂, 10-30% ethyl acetate/hexane gradient) to provide 0.044 g (18%) of the faster eluting exo-isomer and 0.145 g (61%) of the slower eluting endo-isomer. MS (DCI/NH₃) m/z 323, 325 (M+H)⁺.

### Example 46C

### (endo)-4-(6-chloro-3-pyridinyl)octahydrocyclopenta[c]pyrrole 4-methylbenzenesulfonate

The endo-isomer from Example 46B (0.094 g, 0.292 mmol) in EtOAc (5 mL) was treated with p-toluenesulfonic acid monohydrate (0.074 g, 0.39 mmol). The solution was warmed to reflux for 4 hours and then allowed to cool to ambient temperature. The solid was collected by filtration (EtOAc wash) and dried under high vacuum to afford the title compound as a white solid (107 mg, 71%). ¹H NMR (CD₃OD, 300 MHz) δ 1.76 (m, 1H), 1.90-2.10 (m, 3H), 2.37 (s, 3H), 2.50 (dd, J=9.7, 11.5 Hz, 1H), 2.86 (dd, J=7.8, 11.6 Hz, 1H), 2.99-312 (m, 2H), 3.27 (m, 1H), 3.41 (m, 1H), 3.64 (dd, J=8.4, 11.0 Hz, 1H), 7.23 (d, J=7.8 Hz, 2H), 7.42 (d, J=8.3 Hz, 1H), 7.70 (d, J=8.1 Hz, 2H), 7.76 (m, 1H), 8.32 (m, 1H); MS (DCI/NH₃) m/z 223, 225 (M+H)⁺; Anal. calculated for C₁₂H₁₅ClN₂.1.2C₇H₈O₃S: C, 57.07; H, 5.77; N, 6.52. Found: C, 56.92; H, 5.79; N, 6.57.

### EXAMPLE 47

### (cis)-6-(6-chloro-3-pyridinyl)-3-azabicyclo[3.2.0]hept-6-ene 4-methylbenzenesulfonate

### Example 47A

### (cis)-3-[(4-methylphenyl)sulfonyl]-3-azabicyclo[3.2.0]heptan-6-ol

(cis)-3-[(4-Methylphenyl)sulfonyl]-3-azabicyclo[3.2.0]heptan-6-one (8.55 g, 32.2 mmol), prepared according to Gobeaux and Ghosez, Heterocyles, (1989) 28(1), 29-32, in 2:1 THF/CH₂Cl₂ (150 mL) at 0° C. was treated with a 2M solution of LiBH₄ (19.3 mL, 38.7 mmol). After 0.5 hours, the reaction mixture was carefully quenched with 2-propanol (10 mL), allowed to warm to ambient temperature and the volatiles removed under reduced pressure. The residue was diluted with water and extracted twice with CH₂Cl₂. The combined organic extracts were washed with brine, dried over Na₂SO₄, filtered, and the filtrate concentrated under reduced pressure to afford the title compound as a white solid (8.20 g, 95%).

### Example 47B

### (cis)-tert-butyl 6-hydroxy-3-azabicyclo[3.2.0]heptane-3-carboxylate

A solution of sodium napthalenide was prepared according to the procedure described by Heathcock et. al., J. Org. Chem. (1989), 54, 1548-1562 by adding naphthalene (15.4 g, 120 mmol) to a suspension of finely cut sodium metal (2.30 g, 100 mmol) in dimethoxyethane (100 mL), and stirring the resulting dark green mixture at room temperature for 2 hours. The product from Example 47A (8.10 g, 30.3 mmol) in 100 mL of DME at −78° C. was treated slowly with the sodium napthalenide solution (˜65 mL) until a light green color persisted. The reaction mixture was then quenched at −78° C. by the addition of 200 mL of water. The mixture was allowed to warm to 0° C. and di-tert-butyl dicarbonate (6.94 g, 31.8 mmol) was added. After 0.5 hours the mixture was diluted with EtOAc and the layers were separated. The organic extract was washed with brine, dried over Na₂SO₄, filtered and the filtrate concentrated under reduced pressure. The residue was purified by chromatography (5 to 30% EtOAc/CH₂Cl₂ gradient) to afford the title compound as a white solid (5.26 g, 81%). MS (DCI/NH₃) m/z 214 (M+H)⁺.

### Example 47C

### (cis)-tert-butyl 6-oxo-3-azabicyclo[3.2.0]heptane-3-carboxylate

DMSO (4.15 mL, 58.5 mmol) was added dropwise to a solution of oxalyl chloride (2.55 mL, 29.3 mmol) in CH₂Cl₂ (100 mL) at −78° C. (gas evolution). After 15 minutes, a solution of the product from Example 47B (5.20 g, 24.4 mmol) in CH₂Cl₂ (20 mL) was added dropwise. After 30 minutes, triethylamine was added dropwise and the white mixture was stirred at −78° C. for 6 hours then allowed to warm to 40° C. for an additional hour. The mixture was then diluted with water and allowed to warm to room temperature. The layers were separated and the organic extract was washed with water and brine, dried over Na₂SO₄ filtered and and the filtrate concentrated under reduced pressure. The residue was purified by chromatography (10 to 20% EtOAc/CH₂Cl₂ gradient) to afford the title compound as a viscous oil (4.05 g, 79%). MS (DCI/NH₃) m/z 212 (M+H)⁺.

### Example 47D

### (cis)-tert-butyl 6-(6-chloro-3-pyridinyl)-6-hydroxy-3-azabicyclo[3.2.0]heptane-3-carboxylate

5-Bromo-2-chloropyridine (0.888 g, 4.62 mmol) and the product from Example 47C (0.750 g, 3.55 mmol) were processed as described in Example 1E to afford the title compound (1.15 g, 66%). MS (DCI/NH₃) m/z 325, 327 (M+H)⁺

### Example 47E

### (cis)-tert-butyl 6-(6-chloro-3-pyridinyl)-3-azabicyclo[3.2.0]hept-6-ene-3-carboxylate

The product from Example 47D (0.400 g, 1.23 mmol), triethylamine (0.36 mL, 2.6 mmol), methanesulfonyl chloride (0.19 mL, 2.5 mmol), and 4-dimethylaminopyridine (3 mg) were combined in THF and heated at reflux for 16 hours. The solvent was removed under reduced pressure and the residue was purified by chromatography (10% EtOAc/CH₂Cl₂) to afford the title compound as a white foam (0.280 mg, 74%). MS (DCI/NH₃) m/z 307, 309 (M+H)⁺.

### Example 47F

### (cis)-6-(6-chloro-3-pyridinyl)-3-azabicyclo[3.2.0]hept-6-ene 4-methylbenzenesulfonate

The product from Example 47E (0.160 g, 0.522 mmol) was processed as described in Example 46C to provide the title compound as a white solid (0.122 mg, 62%). ¹H NMR (CD₃OD, 300 MHz) δ 2.36 (s, 3H), 3.00-3.15 (m, 2H), 3.40 (d, J=11.9 Hz, 1H), 3.51 (d, J=12.5 Hz, 1H), 3.67 (dd, J=3.4, 6.4 Hz, 1H), 4.03 (dd, J=3.7, 6.5 Hz, 1H), 6.48 (s, 1H), 7.23 (d, J=7.8 Hz, 2H), 7.42 (dd, J=0.7, 8.5 Hz, 1H), 7.70 (d, J=8.1 Hz, 2H), 7.89 (dd, J=2.4, 8.5 Hz, 1H), 8.47 (d, J=2.4 Hz, 1H); MS (DCI/NH₃) m/z 207, 209 (M+H)⁺; Anal. Calculated for C₁₁H₁₁ClN₂.C₇H₈O₃S: C, 57.06; H, 5.05; N 7.39. Found: C, 56.79; H, 4.93; N, 7.24.

### EXAMPLE 48

### (endo)-6-(6-chloro-3-pyridinyl)-3-azabicyclo[3.2.0]heptane 4-methylbenzenesulfonate

### Example 48A

### (endo)-tert-butyl 6-(6-chloro-3-pyridinyl)-3-azabicyclo[3.2.0]heptane-3-carboxylate

### (exo)-tert-butyl 6-(6-chloro-3-pyridinyl)-3-azabicyclo[3.2.0]heptane-3-carboxylate

The product from Example 47D (0.250 g, 0.770 mmol) was processed as described in Example 36A. The residue was purified by chromatography (SiO₂, 5-20% ethyl acetate/CH₂Cl₂ gradient) to provide 0.035 g (15%) of the faster eluting exo-isomer and 0.131 g (55%) of the slower eluting endo-isomer. exo-isomer: ¹H NMR (CDCl₃, 300 MHz) δ 1.51 (s, 9H), 2.18-2.36 (m, 2H), 2.84-3.02 (m, 2H), 3.24-3.33 (m, 2H), 3.44 (m, 1H), 3.60-3.74 (m, 2H), 7.29 (d, J=8.1 Hz, 1H), 7.53 (dd, J=2.2, 8.4 Hz, 1H), 8.25 (d, J=2.0 Hz, 1H); MS (DCI/NH₃) m/z 309, 311 (M+H)⁺. endo-isomer: ¹H NMR (CDCl₃, 300 MHz) δ 1.42 (s, 9H), 2.07 (m, 1H), 2.58 (m, 1H), 3.01 (m, 1H), 3.01-3.32 (m, 4H), 3.60 (br m, 1H), 3.70 (m, 1H), 7.28 (d, J=8.5 Hz, 1H), 7.43 (br d, J=7.4 Hz, 1H), 8.25 (br s, 1H); MS (DCI/NH₃) m/z 309, 311 (M+H)⁺.

### Example 48B

### (endo)-6-(6-chloro-3-pyridinyl)-3-azabicyclo[3.2.0]heptane 4-methylbenzenesulfonate

The endo-isomer from Example 48A (0.130 g, 0.421 mmol) was processed as described in Example 46C to afford the title compound as a white solid (0.135 g, 84%). ¹H NMR (CD₃OD, 300 MHz) δ 2.19 (m, 1H), 2.37 (s, 3H), 2.68, (m, 1H), 3.08 (dd, J=3.9, 13.1 Hz, 1H), 3.25 (m, 1H), 3.30-3.41 (m, 3H), 3.52 (m, 1H), 3.96 (br q, J=9.5 Hz, 1H), 7.23 (d, J=7.8 Hz, 2H), 7.44 (d, J=8.3 Hz, 1H), 7.63 (dd, J=3.7, 8.1 Hz, 1H), 7.69 (d, J=8.2 Hz, 2H), 8.17 (d, J=2.4 Hz, 1H); MS (DCI/NH₃) m/z 209, 211 (M+H)⁺; Anal. Calculated for C₁₁, H₁₃ClN₂.C₇H₈O₃S: C, 56.76; H, 5.56; N 7.35. Found: C, 56.75; H, 5.49; N, 7.26.

### EXAMPLE 49

### (exo)-6-(6-chloro-3-pyridinyl)-3-azabicyclo[3.2.0]heptane 4-methylbenzenesulfonate

The exo-isomer from Example 48A (0.035 g, 0.125 mmol) was processed as described in Example 46C to afford the title compound as a white solid (0.026 g, 60%). ¹H NMR (CD₃OD, 300 MHz) δ 2.25-2.50 (m, 3H), 2.36 (s, 3H), 3.26-3.36 (m, 4H), 3.61 (br d, J=11.2 Hz, 1H), 3.61 (br d, J=11.2 Hz, 1H), 7.23 (d, J=7.8 Hz, 2H), 7.46 (d, J=8.4 Hz, 1H), 7.70 (d, J=8.2 Hz, 2H), 7.84 (m, 1H), 8.27 (d, J=2.4 Hz, 1H); MS (DCI/NH₃) m/z 209, 211 (M+H)⁺; Anal. Calculated for C₁₁H₁₃ClN₂.C₇H₈O₃S: C, 56.76; H, 5.56; N 7.35. Found: C, 56.45; H, 5.52; N, 7.11.

### EXAMPLE 50

### (cis)-6-(5,6-dichloro-3-pyridinyl)-3-azabicyclo[3.2.0]hept-6-ene 4-methylbenzenesulfonate

### Example 50A

### (cis)-tert-butyl 6-(5,6-dichloro-3-pyridinyl)-6-hydroxy-3-azabicyclo[3.2.0]heptane-3-carboxylate

2,3-Dichloro-5-bromopyridine (0.758 g, 2.77 mmol) and the product from Example 47A (0.450 g, 2.13 mmol) were processed as described in Example 1E to afford the title compound (0.565 g, 74%). MS (DCI/NH₃) m/z 359, 361 (M+H)⁺.

### Example 50B

### (cis)-tert-butyl 6-(5,6-dichloro-3-pyridinyl)-3-azabicyclo[3.2.0]hept-6-ene-3-carboxylate

The product from Example 50A (0.359 g, 1.00 mmol) was processed as described in Example 47E to afford the title compound as a white solid (0.131 g, 38%). MS (DCI/NH₃) m/z 341, 343 (M+H)⁺

### Example 50C

### (cis)-6-(5,6-dichloro-3-pyridinyl)-3-azabicyclo[3.2.0]hept-6-ene 4-methylbenzenesulfonate

The product from Example 50B (0.125 g, 1.00 mmol) was processed as described in Example 46C to afford the title compound as a white solid (0.108 g, 72%). ¹H NMR (CD₃OD, 300 MHz) δ 2.37 (s, 3H), 3.02-3.13 (m, 2H), 3.41 (d, J=12.2 Hz, 1H), 3.52 (d, J=12.6 Hz, 1H), 3.68 (dd, J=3.7, 6.4 Hz, 1H), 4.03 (dd, J=3.8, 6.5 Hz, 1H), 6.54 (s, 1H), 7.23 (d, J=8.1 Hz, 2H), 7.68 (d, J=8.1 Hz, 2H), 8.08 (d, J=2.0 Hz, 1H), 8.42 (d, J=2.0 Hz, 1H); MS (DCI/NH₃) m/z 241, 243 (M+H)⁺; Anal. Calculated for C₁₁H₁₀Cl₂N₂.C₇H₈O₃S: C, 52.31; H, 4.39; N, 6.78. Found: C, 52.25; H, 4.52; N, 6.62.

### EXAMPLE 51

### (endo)-6-(5,6-dichloro-3-pyridinyl)-3-azabicyclo[3.2.0]heptane 4-methylbenzenesulfonate

### Example 51A

### (endo)-tert-butyl 6-(5,6-dichloro-3-pyridinyl)-3-azabicyclo[3.2.0]heptane-3-carboxylate

### (exo)-tert-butyl 6-(5,6-dichloro-3-pyridinyl)-3-azabicyclo[3.2.0]heptane-3-carboxylate

The product from Example 50A (0.185 g, 0.515 mmol) was processed as described in Example 36A. The residue was purified by chromatography (SiO₂, 5-20% ethyl acetate/CH₂Cl₂ gradient) to provide 0.018 g (10%) of the faster eluting exo-isomer and 0.80 g (45%) of the slower eluting endo-isomer. Data for the exo-isomer: ¹H NMR (CDCl₃, 300 MHz) δ 1.51 (s, 9H), 2.18-2.36 (m, 2H), 2.90-3.02 (m, 2H), 3.24-3.33 (m, 2H), 3.44 (m, 1H), 3.60-3.72 (m, 2H), 7.65 (d, J=2.2 Hz, 1H), 8.15 (d, J=2.0 Hz, 1H); MS (DCI/NH₃) m/z 343, 345 (M+H)⁺. Data for the endo-isomer: ¹H NMR (CDCl₃, 300 MHz) δ 1.44 (br s, 9H), 2.07 (m, 1H), 2.59 (m, 1H), 3.02 (m, 1H), 3.03-3.32 (m, 4H), 3.58 (br s, 1H), 3.70 (br q, J=9.8 Hz, 1H), 7.57 (br s, 1H), 8.03 (br s, 1H); MS (DCI/NH₃) m/z 343, 345 (M+H)⁺.

### Example 51B

### (endo)-6-(5,6-dichloro-3-pyridinyl)-3-azabicyclo[3.2.0]heptane 4-methylbenzenesulfonate

The endo-isomer from Example 51A (0.077 g, 0.22 mmol) was processed as described in Example 46C to afford 0.066 g (71%) of the title compound as a white solid. ¹H NMR (CD₃OD, 300 MHz) δ 2.18 (m, 1H), 2.37 (s, 3H), 2.68, (m, 1H), 3.08 (dd, J=3.7, 13.2 Hz, 1H), 3.22 (m, 1H), 3.30-3.42 (m, 3H), 3.53 (m, 1H), 3.97 (br q, J=9.5 Hz, 1H), 7.23 (d, J=7.8 Hz, 2H), 7.70 (d, J=8.5 Hz, 2H), 7.82 (d, J=2.1 Hz, 1H), 8.14 (d, J=2.0 Hz, 1H); MS (DCI/NH₃) m/z 243, 245 (M+H)⁺; Anal. Calculated for C₁₁H₁₂Cl₂N₂—C₇H₈O₃S: C, 52.05; H, 4.85; N, 6.74. Found: C, 52.11; H, 4.70; N, 6.63.

It is understood that the foregoing detailed description and Examples are merely illustrative and are not to be taken as limitations upon the scope of the invention, which is defined by the appended claims. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound of formula (I) 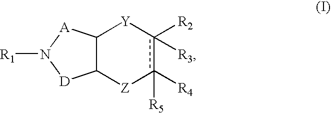
or a pharmaceutically acceptable salt, amide, ester or prodrug thereof, wherein [symbol]
represents a single bond or a double bond;
A is selected from the group consisting of a covalent bond and CH₂;
D is selected from the group consisting of CH₂, CH₂CH₂ and CH₂CH₂CH₂, provided that when D is CH₂CH₂CH₂, then A is a covalent bond;
Y is selected from the group consisting of a covalent bond, CH₂, and CH₂CH₂;
Z is selected from the group consisting of a covalent bond, CH₂, and CH₂CH₂, provided that when Z is CH₂CH₂, then Y is a covalent bond, and further provided that when Y is CH₂CH₂, then Z is a covalent bond;
R₁ is selected from the group consisting of hydrogen, alkoxycarbonyl, alkyl, benzyloxycarbonyl, cyanoalkyl, dihydro-3-pyridinylcarbonyl, hydroxy, hydroxyalkyl, phenoxycarbonyl, —NR₁₀R₁₁, (NR₁₀R₁₁)alkyl and (NR₁₀R₁₁)carbonylalkyl wherein R₁₀ and R₁₁ are independently selected from the group consisting of hydrogen, alkyl and alkylcarbonyl;
R₂ and R₄ are independently selected from the group consisting of hydrogen, aryl and heterocycle, provided that one of R₂ or R₄ is hydrogen; and
R₃ and R₅ are both absent or are independently selected from the group consisting of hydrogen, alkoxy and hydroxy;
provided that when A is a covalent bond, D is CH₂ and Y is a covalent bond, then Z is other than a covalent bond; and
further provided that when A is a covalent bond, D is CH₂ and Z is a covalent bond, then Y is other than a covalent bond.

2. A compound according to claim 1 of formula (II) 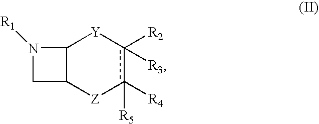
or a pharmaceutically acceptable salt, amide, ester or prodrug thereof.

3. A compound according to claim 2 wherein Y is a covalent bond and Z is CH₂.

4. A compound according to claim 2 wherein Y is CH₂ and Z is a covalent bond.

5. A compound according to claim 2 wherein Y is a covalent bond and Z is CH₂CH₂.

6. A compound according to claim 2 wherein Y is CH₂ and Z is CH₂.

7. A compound according to claim 2 wherein Y is CH₂CH₂ and Z is a covalent bond.

8. A compound according to claim 1 of formula (III) 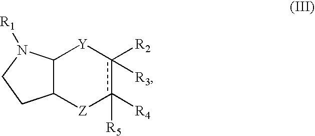
or a pharmaceutically acceptable salt, amide, ester or prodrug thereof.

9. A compound according to claim 8 wherein Y is a covalent bond and Z is a covalent bond.

10. A compound according to claim 8 wherein Y is a covalent bond and Z is CH₂.

11. A compound according to claim 8 wherein [symbol]
represents a double bond;
Y is a covalent bond;
Z is CH₂;
R₂ is hydrogen;
R₃ and R₅ are absent; and
R₄ is heterocycle.

12. A compound according to claim 11 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

13. A compound according to claim 11 wherein the heterocycle is selected from the group consisting of imidazolyl, isoxazolyl, pyridinyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

14. A compound according to claim 11 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

15. A compound according to claim 14 that is (cis)-5-(3-pyridinyl)-1,2,3,3a,4,6a-hexahydrocyclopenta[b]pyrrole.

16. A compound according to claim 8 wherein Y is CH₂ and Z is a covalent bond.

17. A compound according to claim 8 wherein [symbol]
represents a double bond;
Y is CH₂;
Z is a covalent bond;
R₂ is heterocycle;
R₃ and R₅ are absent; and
R₄ is hydrogen.

18. A compound according to claim 17 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

19. A compound according to claim 17 wherein the heterocycle is selected from the group consisting of imidazolyl, isoxazolyl, pyridinyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

20. A compound according to claim 17 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

21. A compound according to claim 20 that is (cis)-5-(3-pyridinyl)-1,2,3,3a,6,6a-hexahydrocyclopenta[b]pyrrole.

22. A compound according to claim 8 wherein Y is a covalent bond and Z is CH₂CH₂.

23. A compound according to claim 8 wherein Y is CH₂ and Z is CH₂.

24. A compound according to claim 8 wherein Y is CH₂CH₂ and Z is a covalent bond.

25. A compound according to claim 1 of formula (IV) 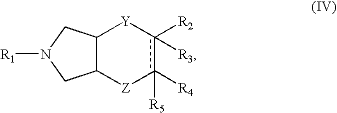
or a pharmaceutically acceptable salt, amide, ester or prodrug thereof.

26. A compound according to claim 25 wherein Y is a covalent bond and Z is a covalent bond.

27. A compound according to claim 25 wherein [symbol]
represents a double bond;
Y is a covalent bond;
Z is a covalent bond;
R₂ is hydrogen;
R₃ and R₅ are absent; and
R₄ is heterocycle.

28. A compound according to claim 27 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

29. A compound according to claim 27 wherein the heterocycle is selected from the group consisting of imidazolyl, isoxazolyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

30. A compound according to claim 27 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

31. A compound according to claim 30 selected from the group consisting of
(cis)-6-(6-methyl-3-pyridinyl)-3-azabicyclo[3.2.0]hept-6-ene; and
cis)-6-(5,6-dichloro-3-pyridinyl)-3-azabicyclo[3.2.0]hept-6-ene.

32. A compound according to claim 25 wherein [symbol]
represents a single bond;
Y is a covalent bond;
Z is a covalent bond;
R₂, R₃ and R₅ are hydrogen; and
R₄ is heterocycle.

33. A compound according to claim 32 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

34. A compound according to claim 32 wherein the heterocycle is selected from the group consisting of imidazolyl, isoxazolyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

35. A compound according to claim 32 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

36. A compound according to claim 35 selected from the group consisting of
(exo)-6-(6-chloro-3-pyridinyl)-3-azabicyclo[3.2.0]heptane;
(exo)-6-(6-chloro-3-pyridinyl)-3-azabicyclo[3.2.0]heptane; and
(endo)-6-(5,6-dichloro-3-pyridinyl)-3-azabicyclo[3.2.0]heptane.

37. A compound according to claim 25 wherein Y is a covalent bond and Z is CH₂.

38. A compound according to claim 25 wherein [symbol]
represents a double bond;
Y is a covalent bond;
Z is CH₂;
R₂ is hydrogen;
R₃ and R₅ are absent; and
R₄ is heterocycle.

39. A compound according to claim 38 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

40. A compound according to claim 38 wherein the heterocycle is selected from the group consisting of imidazolyl, isoxazolyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

41. A compound according to claim 40 selected from the group consisting of
(cis)-5-(3-methyl-5-isoxazolyl)-1,2,3,3a,4,6a-hexahydrocyclopenta[c]pyrrole;
(cis)-2-methyl-5-(3-methyl-5-isoxazolyl)-1,2,3,3a,4,6a-hexahydrocyclopenta[c]pyrrole;
(cis)-5-(3-bromo-1,2,4-thiadiazol-5-yl)-1,2,3,3a,4,6a-hexahydrocyclopenta[c]pyrrole;
(cis)-5-(1,3-thiazol-2-yl)-1,2,3,3a,4,6a-hexahydrocyclopenta[c]pyrrole;
(cis)-5-(3,5-dimethyl-4-isoxazolyl)-1,2,3,3a,4,6a-hexahydrocyclopenta[c]pyrrole;
(cis)-5-(1H-imidazol-4-yl)-1,2,3,3a,4,6a-hexahydrocyclopenta[c]pyrrole;
(cis)-5-(1,3-thiazol-5-yl)-1,2,3,3a,4,6a-hexahydrocyclopenta[c]pyrrole;
(cis)-5-(imidazo[1,2-a]pyridin-3-yl)-1,2,3,3a,4,6a-hexahydrocyclopenta[c]pyrrole;
(cis)-5-(imidazo[1,2-a]pyridin-6-yl)-1,2,3,3a,4,6a-hexahydrocyclopenta[c]pyrrole;
(cis)-5-(thieno[3,2-b]pyridin-2-yl)-1,2,3,3a,4,6a-hexahydrocyclopenta[c]pyrrole;
(cis)-5-(1-trityl-1H-imidazol-4-yl)-1,2,3,3a,4,6a-hexahydrocyclopenta[c]pyrrole;
(cis)-5-(5-(aminosulfonyl)-2-thienyl)-1,2,3,3a,4,6a-hexahydrocyclopenta[c]pyrrole;
(cis)-5-(5-(amino(imino)methyl)-2-thienyl)-1,2,3,3a,4,6a-hexahydrocyclopenta[c]pyrrole;
(cis)-5-(2-methyl-2H-tetrazol-5-yl)-1,2,3,3a,4,6a-hexahydrocyclopenta[c]pyrrole;
(cis)-5-(thieno[2,3-b]pyridin-5-yl)-1,2,3,3a,4,6a-hexahydrocyclopenta[c]pyrrole;
(cis)-5-(imidazo[1,2-a]pyridin-7-yl)-1,2,3,3a,4,6a-hexahydrocyclopenta[c]pyrrole;
(cis)-5-(5-nitro-1,3-thiazol-2-yl)-1,2,3,3a,4,6a-hexahydrocyclopenta[c]pyrrole; and
(cis)-5-(1,3,4-thiadiazol-2-yl)-1,2,3,3a,4,6a-hexahydrocyclopenta[c]pyrrole.

42. A compound according to claim 38 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

43. A compound according to claim 42 selected from the group consisting of
(cis)-5-(6-chloro-3-pyridinyl)-1,2,3,3a,4,6a-hexahydrocyclopenta[c]pyrrole;
(cis)-5-(5-methoxy-3-pyridinyl)-1,2,3,3a,4,6a-hexahydrocyclopenta[c]pyrrole;
(cis)-5-(6-chloro-5-methyl-3-pyridinyl)-1,2,3,3a,4,6a-hexahydrocyclopenta[c]pyrrole;
(cis)-5-(3-pyridinyl)-1,2,3,3a,4,6a-hexahydrocyclopenta[c]pyrrole;
(cis)-5-(5,6-dichloro-3-pyridinyl)-1,2,3,3a,4,6a-hexahydrocyclopenta[c]pyrrole;
(cis)-5-(5-chloro-3-pyridinyl)-1,2,3,3a,4,6a-hexahydrocyclopenta[c]pyrrole;
(cis)-5-(6-methyl-3-pyridinyl)-1,2,3,3a,4,6a-hexahydrocyclopenta[c]pyrrole;
(cis)-5-(2-methyl-3-pyridinyl)-1,2,3,3a,4,6a-hexahydrocyclopenta[c]pyrrole;
(cis)-5-(6-chloro-5-fluoro-3-pyridinyl)-1,2,3,3a,4,6a-hexahydrocyclopenta[c]pyrrole;
(cis)-5-(6-chloro-3-pyridinyl)-2-cyanomethyl-1,2,3,3a,4,6a-hexahydrocyclopenta[c]pyrrole;
(cis)-5-(6-fluoro-3-pyridinyl)-1,2,3,3a,4,6a-hexahydrocyclopenta[c]pyrrole;
(cis)-5-(2-pyridinyl)-1,2,3,3a,4,6a-hexahydrocyclopenta[c]pyrrole;
(cis)-5-(4-pyridinyl)-1,2,3,3a,4,6a-hexahydrocyclopenta[c]pyrrole;
(cis)-5-(6-methyl-2-pyridinyl)-1,2,3,3a,4,6a-hexahydrocyclopenta[c]pyrrole;
(cis)-5-(5-bromo-3-pyridinyl)-1,2,3,3a,4,6a-hexahydrocyclopenta[c]pyrrole;
(cis)-5-(5-vinyl-3-pyridinyl)-1,2,3,3a,4,6a-hexahydrocyclopenta[c]pyrrole;
(cis)-5-(1,2,3,3a,4,6a-hexahydrocyclopenta[c]pyrrol-5-yl)nicotinonitrile;
(3aS,6aR)-5-(6-chloro-3-pyridinyl)-1,2,3,3a,4,6a-hexahydrocyclopenta[c]pyrrole; and
(3 aR,6aS)-5-(6-chloro-3-pyridinyl)-1,2,3,3a,4,6a-hexahydrocyclopenta[c]pyrrole.

44. A compound according to claim 25 wherein [symbol]
represents a single bond;
Y is a covalent bond;
z is CH₂;
R₂ and R₃ are hydrogen;
R₅ is selected from the group consisting of hydrogen and hydroxy; and
R₄ is heterocycle.

45. A compound according to claim 44 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₄, —C(NOR₁₂)R₁₃, —C(NCN)R₁₂, —C(NNR₁₂R₁₃)R₁₄, —S(O)₂OR₁₂, and —S(O)₂R₁₂.

46. A compound according to claim 44 wherein the heterocycle is selected from the group consisting of imidazolyl, isoxazolyl, pyridinyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

47. A compound according to claim 44 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

48. A compound according to claim 47 selected from the group consisting of
(cis)-5-(3-pyridinyl)octahydrocyclopenta[c]pyrrole;
(endo)-5-(6-chloro-3-pyridinyl)octahydrocyclopenta[c]pyrrole;
(exo)-5-(6-chloro-3-pyridinyl)octahydrocyclopenta[c]pyrrole; and
(cis)-5-(6-chloro-3-pyridinyl)octahydrocyclopenta[c]pyrrol-5-ol.

49. A compound according to claim 25 wherein [symbol]
represents a double bond;
Y is a covalent bond;
Z is CH₂;
R₂ is heterocycle;
R₃ and R₅ are absent; and
R₄ is hydrogen.

50. A compound according to claim 49 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

51. A compound according to claim 49 wherein the heterocycle is selected from the group consisting of imidazolyl, isoxazolyl, pyridinyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

52. A compound according to claim 49 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

53. A compound according to claim 52 that is (cis)-6-(6-chloro-3-pyridinyl)-1,2,3,3a,4,6a-hexahydrocyclopenta[c]pyrrole.

54. A compound according to claim 25 wherein [symbol]
represents a single bond;
Y is a covalent bond;
Z is CH₂;
R₂ is heterocycle;
R₃ is selected from the group consisting of hydrogen and hydroxy; and
R₄ and R₅ are hydrogen.

55. A compound according to claim 54 wherein the heterocycle is selected from the group consisting of furyl, imidazolyl, isothiazolyl, isoxazolyl, oxadiazolyl, oxazolyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothienyl, benzoxazolyl, benzofuranyl, cinnolinyl, furo[2,3-c]pyridine, furo[3,2-c]pyridine, furo[3,2-b]pyridinyl, furo[2,3-b]pyridinyl, imidazo[1,2-a]pyridinyl, indazolyl, indolyl, indolizinyl, naphthyridinyl, isobenzofuranyl, isobenzothienyl, isoindolyl, isoquinolinyl, phthalazinyl, quinolinyl, quinolizinyl, quinoxalinyl, quinazolinyl, thieno[2,3-c]pyridine, thieno[3,2-c]pyridine, thieno[3,2-b]pyridinyl, and thieno[2,3-b]pyridinyl wherein the heterocycle is substituted with 0, 1, 2, or 3 substituents independently selected from the group consisting of alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, triphenylmethyl (trityl), —C(NH)NR₁₀R₁₁, —NR₁₀R₁₁, (NR₁₀R₁₁)alkyl, (NR₁₀R₁₁)carbonyl, (NR₁₀R₁₁)carbonylalkyl, (NR₁₀R₁₁)sulfonyl, —NR₁₂S(O)₂R₁₃, —C(NR₁₂)NR₁₃R₁₄, —CH₂C(NR₁₂)NR₁₃R₁₄, —C(NOR₁₂)R₁₃, —C(NCN)R₁₂, —C(NNR₁₂R₁₃)R₁₄, —S(O)₂OR₁₂, and —S(O)₂R₁₂.

56. A compound according to claim 54 wherein the heterocycle is selected from the group consisting of imidazolyl, isoxazolyl, pyridinyl, tetrazolyl, thiadiazolyl, thiazolyl, thienyl, imidazo[1,2-a]pyridinyl, thieno[3,2-b]pyridinyl, and thieno[2,3-b]pyridinyl wherein the heterocycle is substituted with 0, 1, or 2 substituents independently selected from the group consisting of alkenyl, alkenyloxy, alkoxy, alkyl, alkynyl, cyano, halogen, nitro, (NR₁₀R₁₁)sulfonyl, and —C(NH)NR₁₀R₁₁.

57. A compound according to claim 54 wherein the heterocycle is pyridinyl substituted with 0, 1, or 2 substituents independently selected from the group consisting of alkenyl, alkenyloxy, alkoxy, alkyl, alkynyl, cyano, halogen, and nitro.

58. A compound according to claim 57 selected from the group consisting of
(cis)-4-(3-pyridinyl)octahydrocyclopenta[c]pyrrol-4-ol; and
(endo)-4-(6-chloro-3-pyridinyl)octahydrocyclopenta[c]pyrrole.

59. A compound according to claim 25 wherein Y is a covalent bond and Z is CH₂CH₂.

60. A compound according to claim 25 wherein Y is CH₂ and Z is CH₂.

61. A compound according to claim 1 of formula (V) 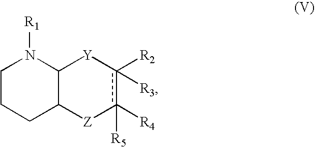
or a pharmaceutically acceptable salt, amide, ester or prodrug thereof thereof.

62. A compound according to claim 61 wherein Y is a covalent bond and Z is a covalent bond.

63. A compound according to claim 61 wherein Y is a covalent bond and Z is CH₂.

64. A compound according to claim 61 wherein Y is CH₂ and Z is a covalent bond.

65. A compound according to claim 61 wherein Y is a covalent bond and Z is CH₂CH₂.

66. A compound according to claim 61 wherein Y is CH₂ and Z is CH₂.

67. A compound according to claim 61 wherein Y is CH₂CH₂ and Z is a covalent bond.

68. A compound according to claim 1 of formula (VI) 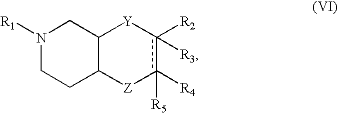
or a pharmaceutically acceptable salt, amide, ester or prodrug thereof thereof.

69. A compound according to claim 68 wherein Y is a covalent bond and Z is a covalent bond.

70. A compound according to claim 68 wherein Y is a covalent bond and Z is CH₂.

71. A compound according to claim 68 wherein Y is CH₂ and Z is a covalent bond.

72. A compound according to claim 68 wherein Y is a covalent bond and Z is CH₂CH₂.

73. A compound according to claim 68 wherein Y is CH₂ and Z is CH₂.

74. A compound according to claim 68 wherein Y is CH₂CH₂ and Z is a covalent bond.

75. A pharmaceutical composition comprising a therapeutically effective amount of a compound of formula (I) or a pharmaceutically acceptable salt, amide, ester or prodrug thereof in combination with a pharmaceutically acceptable carrier.

76. A method of treating a disorder wherein the disorder is selected from the group consisting of Alzheimer's disease, Parkinson's disease, attention deficit hyperactivity disorder, depression, nicotinic withdrawal syndrome, Tourette's syndrome and schizophrenia in a mammal comprising administering to the mammal in need of such treatment a therapeutically effective amount of a compound of formula (I) or a pharmaceutically acceptable salt, amide, ester or prodrug thereof.

77. A method of treating pain in a mammal comprising administering to the mammal in need of such treatment a therapeutically effective amount of a compound of formula (I) or a pharmaceutically acceptable salt, amide, ester or prodrug thereof.

78. A method of treating pain in a mammal comprising administering to the mammal in need of such treatment a therapeutically effective amount of a compound of formula (I) or a pharmaceutically acceptable salt, amide, ester or prodrug thereof in combination with a non-steroid anti-inflammatory agent.

79. A method of treating pain in a mammal comprising administering to the mammal in need of such treatment a therapeutically effective amount of a compound of formula (I) or a pharmaceutically acceptable salt, amide, ester or prodrug thereof in combination with an opioid.

80. A method of treating pain in a mammal comprising administering to the mammal in need of such treatment a therapeutically effective amount of a compound of formula (I) or a pharmaceutically acceptable salt, amide, ester or prodrug thereof in combination with a tricyclic antidepressant.

81. A method of treating pain in a mammal comprising administering to the mammal in need of such treatment a therapeutically effective amount of a compound of formula (I) or a pharmaceutically acceptable salt, amide, ester or prodrug thereof in combination with an anticonvulsant.
